(19) United States
(12) Patent Application Publication
(10) Pub. No.: US 2023/0174951 A1
(43) Pub. Date: Jun. 8, 2023

(54) PHENOL-RICH GRAPES

(71) Applicants: THE STATE OF ISRAEL, MINISTRY OF AGRICULTURE & RURAL DEVELOPMENT, AGRICULTURAL RESEARCH ORGANIZATION, Rishon-Lezion (IL); B. G. Negev Technologies and Applications Ltd., at Ben-Gurion University, Beer-Sheva (IL)

(72) Inventors: Avihai PERL, Rishon Lezion (IL); Michal OREN-SHAMIR, Rehovot (IL); Miriam FRIEDMAN-EINAT, Ness Ziona (IL); Ru WANG, Rehovot (IL); Aaron FAIT, Mitzpe Ramon (IL)

(73) Assignees: THE STATE OF ISRAEL, MINISTRY OF AGRICULTURE & RURAL DEVELOPMENT, AGRICULTURAL RESEARCH ORGANIZATION, Rishon Lezion (IL); B. G. Negev Technologies and Applications Ltd., at Ben-Gurion University, Beer Sheva (IL)

(21) Appl. No.: 17/921,120

(22) PCT Filed: Apr. 25, 2021
(86) PCT No.: PCT/IL2021/050474

§ 371 Date: Oct. 25, 2022

Related U.S. Application Data
(60) Provisional application No. 63/111,639, filed on Nov. 10, 2020.
(60) Provisional application No. 63/015,575, filed on Apr. 26, 2020.

(51) Int. Cl. C12N 5/14; C12N 15/82; A61P 37/06; C12N 15/52

(52) U.S. Cl. CPC C12N 5/14; A61P 37/06; C12N 15/52; C12N 15/8243

(57) ABSTRACT

The present invention relates to transgenic grape cells, compositions comprising such cells or extracts or fractions thereof, and methods of use thereof for inhibiting or reducing the incidence of cytokine release syndrome or cytokine storm in a subject. Disclosed herein are also methods for preventing, treating, reducing the incidence, suppressing or inhibiting a coronavirus infection or a symptom thereof.

### FIELD OF THE INVENTION

Disclosed herein are transgenic grape cells, compositions comprising such cells or extracts or fractions thereof, and their use for inhibiting or reducing the incidence of cytokine release syndrome or cytokine storm in a subject. Disclosed herein are also methods for preventing, treating, reducing the incidence, suppressing or inhibiting a coronavirus infection or a symptom thereof.

### BACKGROUND

The phenolic content in grape-based wine includes a large group of several hundred chemical compounds that affect the taste, color and mouthfeel of wine. These compounds include phenolic acids, stilbenoids, flavonols, dihydroflavonols, anthocyanins, flavanol monomers and flavanol polymers, and can be broadly separated into two categories, flavonoids and non-flavonoids. Flavonoids include the anthocyanins and tannins which contribute to the color and mouthfeel of the wine, while the non-flavonoids include the stilbenoids such as resveratrol and phenolic acids.

Stilbenes are a small group of phenylpropanoids, characterized by a 1, 2-diphenylethylene backbone, most of which are derivatives from the monomeric unit trans-resveratrol (Chong et al., 2009; Rimando et al., 2012). This unique group of secondary metabolites with phytoalexin characteristics is synthesized in a limited and unrelated number of plant species (Shen et al., 2009). Stilbenes have outstanding pharmacological and nutritional values, and many of the stilbene-producing plants are part of the human diet, including cranberry, peanut, cocoa, and in particular grapes (Counet et al., 2006; Yin et al., 2016). Grapes are the major source of stilbenes in human nutrition, accumulating multiple stilbene-derived compounds, including isomers, polymers and glycosylated forms.

Due to their antimicrobial characteristics, stilbenes accumulate in infected areas of plants following pathogen attack (Ahuja et al., 2012). Among the stilbenes, the resveratrol dehydrodimers viniferins exhibited potent antifungal activity and were present in high concentrations in fungal-resistant varieties of the grape vine. One example comparing susceptible and resistant grape vine varieties showed that fungal lesions (Botrytis cinerea and Plasmopara viticola) of resistant cultivars contained low concentrations of resveratrol and higher concentrations of its oligomers α- and ε-viniferin (Langcake, 1981). A second study on downy mildew in grapevine showed that resveratrol production was induced in all varieties, but was glycosylated into a nontoxic stilbene, piceide (5,4′-dihydroxystilbene-3—O—β-glucopyranoside), in the susceptible varieties, while in the resistant varieties it was oxidized into toxic viniferins (Pezet et al., 2004). Purified ε-viniferin was also shown to be a stronger phytoalexine than resveratrol against Plasmopara viticola and Botrytis cinerea (Chong et al., 2009; Schnee et al., 2013). Due to its strong anti-fungal characteristics in comparison to the well-studied resveratrol, viniferin content is becoming a standard for selecting resistant grape varieties (Gindro et al., 2006).

Resveratrol was shown to have health promoting activities in humans, including anticancer, antioxidant, anti-inflammatory, and neuroprotective characteristics (Baur and Sinclair, 2006; Kalantari and Das, 2010). ε-Viniferins, accumulating in grapes and wine to concentration similar to resveratrol, exhibit even higher pharmacological activities than those of resveratrol (Vitrac et al., 2005). One example is a stronger inhibitory effect of viniferin compared to resveratrol of cytochromes P450 (CYPs) enzyme activities, in cancer prevention (Piver et al., 2003). Another example is inhibition of the onset of Alzheimer’s disease by preventing the extracellular accumulation of aggregated amyloid β peptides in senile plaques: ε-viniferin is more stable metabolically than resveratrol, and therefore more effective in preventing amyloid β aggregation and exerting anti-inflammatory and antioxidant activities (Vion et al., 2018). A third example is in protecting cardiovascular function: Viniferin, unlike resveratrol, was found to inhibit angiotensin-converting enzyme (ACE) activity, an important therapeutic approach for lowering blood pressure and preventing heart failure, and improve cardiac mass in spontaneously hypertensive rats (Zghonda et al., 2012).

Resveratrol has multiple activities against harmful inflammatory cytokines and related microRNA. The anti-inflammatory properties of resveratrol have been studied on animal models, cell lines and human subjects and proven to be very effective in reducing inflammatory cell production and pro-inflammatory cytokine accumulation. (Rafe et al., 2019).

Flavonoids are the most abundant polyphenols in the human diet and are considered health-promoting compounds due to their antioxidant and anti-inflammatory activities. High flavonoid consumption is correlated to prevention of cancers, cardiovascular diseases, Alzheimer’s, and atherosclerosis (Babu et al., 2009, Hollman and Katan, 1999, Kris-Etherton et al., 2004). There are three major flavonoid subgroups in grapes: flavonols, flavan-3-ols (tannin), and anthocyanins (Blancquaert et al., 2019). Many of the health-related properties of flavonoids have been attributed to their flavonol subclass (Owens et al., 2008; Harbome et al., 2000). Among the health benefits of flavonols, kaempferol was found to reduce the risk of chronic diseases including cancer (Chen et al., 2013), quercetin was linked to increasing the lifespan extension in mammals (Haigis et al., 2010) and myricetin was found to reduce the risks of cancer and diabetes (Feng et al., 2015).

Flavonol synthase (FLS) catalyzes the synthesis of the three major flavonols from their dihydroflavonols, kaempferol, quercetin and myricetin. Overexpression of FLS results in increased flavonols levels. One example is overexpression of the Brassica napus FLS in Arabidopsis that resulted in increased kaempferol and quercetin levels (Vu et al., 2015). Overexpression of FLS in tobacco also led to increased kaempferol levels (Jiang et al., 2020). On the other hand, inhibition of FLS in lisianthus plants prevented flavonol biosynthesis (Nielsen et al., 2002).

Plant cell suspensions offer defined production systems, with rapid yield and relatively uniform quality, which are free from geographical, environmental and seasonal constrains, unlike whole plants (Davies and Deroles, 2014). These advantages of plant cell culture make Vitis vinifera cv. Gamey cell suspensions a promising material to produce resveratrol and its derivatives.

Increased availability of Phe has been shown to affect several metabolic pathways derive from Phe, including stilbenes. Overexpression of the feedback-insensitive bacterial form of DAHSP (3-deoxy-D-arabino-heptulosonate 7-phosphate synthase), AroG*, resulted in increased production of aromatic amino acids and in particular Phe in Arabidopsis (Tzin et al., 2012), petunia (Oliva et al., 2015), tomato (Tzin et al., 2015) and the Vitis vinifera cv. Gamay Red cell suspension (Manela et al., 2015). In all cases AroG* overexpression caused increased production of secondary metabolites, differing from one plant to the other. The only stilbene affected by AroG* overexpression in the grape cell suspension was resveratrol, with a 20-fold increase in its concentration (Manela et al., 2015).

A second approach for increasing stilbenes is by overexpressing stilbene synthase (STS) catalyzing the condensation of p-coumaroyl-CoA with three units of malonyl-CoA to produce resveratrol. Similarly, most studies overexpressing STS in plant cell cultures reported on increased production of resveratrol, and only resveratrol were identified (Aleynova et al., 2016; Chu et al., 2017; Hidalgo, 2017; Kiselev and Aleynova, 2016; Suprun et al., 2019). Only one study suggested that viniferin levels increased as well (Suprun et al., 2019).

Diseases such as COVID-19 and influenza can be fatal due to an overreaction of the body’s immune system called a cytokine storm. Cytokine release syndrome (CRS) or cytokine storm syndrome (CSS) is a form of systemic inflammatory response syndrome (SIRS) that can be triggered by a variety of factors, such as viral infection. Severe cases are termed “cytokine storms”. Cytokines are small proteins released by many different cells in the body, including those of the immune system where they coordinate the body’s response against infection and trigger inflammation. Sometimes the body’s response to infection can go into overdrive. For example, when SARS -CoV-2– the virus behind the COVID-19 pandemic – enters the lungs, it triggers an immune response, attracting immune cells to the region to attack the virus, resulting in localized inflammation. But in some patients, excessive or uncontrolled levels of cytokines are released which then activate more immune cells, resulting in hyperinflammation. This can seriously harm or even kill the patient. Cytokine storms are a common complication not only of COVID-19 and flu but of other respiratory diseases caused by coronaviruses such as SARS and MERS. They are also associated with non-infectious diseases such as multiple sclerosis and pancreatitis.(https://www.newscientist.com/term/cytokine-storm/#ixzz6JhDBRH7E)

Currently, there is a high demand for stilbenes due to their valuable pharmacological properties and role as phytoalexins.

### SUMMARY

The present invention provides cells, compositions, and methods to produce phenol-rich cells and compositions. Such phenol-rich products are useful in several fields of human therapy, as the health benefits of plant-based polyphenols are long known.

According to the principles of the present invention, methods are provided to enrich plant cells, especially genetically-modified plant cells, with beneficial polyphenolic compounds, far beyond the polyphenol levels found in nature.

The present invention provides, in one aspect, a doubly-transgenic Vitis Vinifera cell comprising at least one copy of an AroG* gene and at least one copy of a stilbene synthase (STS) gene or a flavonol synthase (FLS) gene.

In certain embodiments, the Vitis Vinifera cell is a Vitis Vinifera cv. Gamay Red cell.

In certain embodiments, the AroG* gene encodes a 3-Deoxy-D-arabinoheptulosonate 7-phosphate (DAHP) synthase (DAHPS) enzyme.

In certain embodiments, the DAHPS enzyme is a feedback-insensitive DAHPS enzyme.

In certain embodiments, the DAHPS enzyme increases the availability of at least one amino-acid in the cell.

In certain embodiments, the DAHPS enzyme increases the availability of Phenylalanine in the cell.

In certain embodiments, the STS gene encodes an STS enzyme.

In certain embodiments, the STS enzyme produces a stilbene.

In certain embodiments, the STS gene is a Vitis vinifera stilbene synthase (VvSTS) gene.

In certain embodiments, the STS gene is selected from the group consisting of VvSTS5, VvSTS10 and VvSTS28.

In certain embodiments, the FLS gene encodes an FLS enzyme.

In certain embodiments, the FLS enzyme produces a flavonoid.

In certain embodiments, the FLS gene is a Vitis vinifera flavonol synthase (VvFLS) gene.

In certain embodiments, the FLS gene is VIT_07s0031g00100.

In certain embodiments, the AroG* gene or the STS gene is functionally-linked to a constitutive promoter.

In certain embodiments, the constitutive promoter is Cauliflower mosaic virus (CaMV) 35S RNA promoter (35S promoter).

In certain embodiments, the AroG* gene and the STS gene are both functionally-linked to a constitutive promoter.

In certain embodiments, the AroG* gene and the STS gene are functionally-linked to different constitutive promoters.

In certain embodiments, the AroG* gene or the FLS gene is functionally-linked to a constitutive promoter.

In certain embodiments, the AroG* gene and the FLS gene are both functionally-linked to a constitutive promoter.

In certain embodiments, the AroG* gene and the STS gene are functionally-linked to different constitutive promoters.

In certain embodiments, the cell described above comprises a higher level of: at least one amino-acid selected from the group consisting of Phenylalanine, Tryptophan, and p-CA; at least one stilbene selected from the group consisting of trans-piceid, cis-piceid, resveratrol, and ε-viniferin; at least one flavonoid selected from the group consisting of quercetin-3-glucoside, myricetin, catechin, epicatechin, epigallocatechin, procyanidin B1 and procyanidin B2; at least one anthocyanin selected from the group consisting of cyanidin, peonidin, delphinidin, petunidin, malvidin, malvidin-3 -o-glucoside, malvidin-3 -com-glucoside, malvidin-3 -acetyl-glucoside, petunidin-3-com-glucoside,; or any combination of the above, compared to a corresponding non-transgenic Vitis Vinifera cell or compared to a corresponding singly-transgenic Vitis Vinifera cell.

In certain embodiments, the cell described above comprises a concentration of at least 0.5 mg/g dry weight trans-piceid, a concentration of at least 0.5 mg/g dry weight cis-piceid, a concentration of at least 0.8 mg/g dry weight resveratrol, a concentration of at least 0.6 mg/g dry weight ε-viniferin, or any combination of the above.

In certain embodiments, the cell described above comprises a similar or lower level of: at least one flavonoid selected from the group consisting of myricetin, quercetin-3-glucoside, catechin, epicatechin, epigallocatechin, and procyanidin; at least one anthocyanin selected from the group consisting of cyanidin, peonidin, delphinidin, petunidin, and malvidin; or any combination of the above, compared to a corresponding non-transgenic Vitis Vinifera cell or compared to a corresponding singly-transgenic Vitis Vinifera cell.

In certain embodiments, the cell described above comprises resveratrol in a concentration of about 1.26 mg/g dry weight, ε-viniferin in a concentration of about 10.8 mg/g dry weight, or both.

The present invention further provides, in another aspect, a method for maintaining a Vitis Vinifera cell optionally comprising at least one copy of an AroG* gene, optionally comprising at least one copy of a stilbene synthase (STS) gene, , and optionally comprising at least one copy of a flavonol synthase (FLS) gene, the method comprising contacting the cell with a composition comprising: phenylalanine in a concentration of about 0.2 mM to about 5 mM, p-coumaric acid in a concentration of about 0.1 mM to about 0.3 mM, or any combination of the above.

In certain embodiments, the cell comprises at least one copy of an AroG* gene.

In certain embodiments, the cell comprises at least one copy of an STS gene.

In certain embodiments, the cell comprises at least one copy of an FLS gene.

In certain embodiments, the method described above comprises contacting the cell with a composition comprising phenylalanine in a concentration of about 2 mM to about 5 mM.

In certain embodiments, the method described above comprises contacting the cell with a composition comprising phenylalanine in a concentration of about 5 mM.

In certain embodiments, the method described above comprises contacting the cell with a composition comprising p-coumaric acid in a concentration of about 0.3 mM.

The present invention further provides, in yet another aspect, a pharmaceutical composition, comprising a doubly-transgenic Vitis Vinifera cell as described above, or an extract or fraction thereof.

In certain embodiments, the doubly-transgenic Vitis Vinifera cell was maintained by the method described above.

The present invention further provides, in yet another aspect, a pharmaceutical composition, comprising a non-transgenic Vitis Vinifera cell or a single-transgenic Vitis Vinifera cell comprising at least one copy of an AroG* gene, wherein the cell was maintained by the method described above, or an extract or fraction thereof.

In certain embodiments, the pharmaceutical composition described above comprises: at least one amino-acid selected from the group consisting of Phenylalanine, Tryptophan, and p-CA; at least one stilbene selected from the group consisting of trans-piceid, cis-piceid, resveratrol, and ε-viniferin; at least one flavonoid selected from the group consisting of quercetin-3-glucoside, myricetin, catechin, epicatechin, epigallocatechin, procyanidin B1 and procyanidin B2; at least one anthocyanin selected from the group consisting of cyanidin, peonidin, delphinidin, petunidin, malvidin, malvidin-3-o-glucoside, malvidin-3-com-glucoside, malvidin-3-acetyl-glucoside, and petunidin-3-com-glucoside; or any combination of the above.

In certain embodiments, the pharmaceutical composition described above comprises an extract of the doubly-transgenic Vitis Vinifera cell or the single-transgenic Vitis Vinifera cell.

In certain embodiments, the pharmaceutical composition described above comprises a cytoplasmic fraction of the doubly-transgenic Vitis Vinifera cell or the single-transgenic Vitis Vinifera cell.

In certain embodiments, the pharmaceutical composition described above comprises a polyphenolic fraction of the doubly-transgenic Vitis Vinifera cell or the single-transgenic Vitis Vinifera cell.

In certain embodiments, the pharmaceutical composition described above comprises the vacuole of the doubly-transgenic Vitis Vinifera cell or the single-transgenic Vitis Vinifera cell.

In certain embodiments, the pharmaceutical composition described above is substantially dehydrated composition.

In certain embodiments, the pharmaceutical composition described above is substantially devoid of intact cells.

In certain embodiments, the pharmaceutical composition described above is substantially devoid of ruptured cells.

The present invention further provides, in yet another aspect, a method of preventing, treating, reducing the incidence, suppressing or inhibiting a Coronavirus infection or a symptom thereof in a patient in need, the method comprising administering to the patient a therapeutically-effective amount of the pharmaceutical composition described above.

In certain embodiments, the symptom is a cytokine storm.

In certain embodiments, the pharmaceutical composition is systemically administered to the patient.

In certain embodiments, the Coronavirus infection comprises Severe acute respiratory syndrome coronavirus 2 (SARS-CoV-2) infection.

In certain embodiments, the pharmaceutical composition is orally administered to the patient.

The present invention further provides, in yet another aspect, a crop plant or part thereof, comprising at least one copy of an AroG* gene and at least one copy of a stilbene synthase (STS) gene or a flavonol synthase (FLS) gene.

In certain embodiments, the crop plant is Vitis Vinifera.

In certain embodiments, the Vitis Vinifera is Gamay Red cultivar.

The present invention further provides, in yet another aspect, a method of treating, preventing, ameliorating, inhibiting, or reducing the incidence of a cytokine release syndrome (CRS) or a Cytokine Storm in a patient in need, the method comprising administering to the patient a therapeutically-effective amount of the pharmaceutical composition described above.

In certain embodiments, the CRS or Cytokine Storm is associated with a Coronavirus infection or with a symptom thereof.

In certain embodiments, the Coronavirus infection comprises Severe acute respiratory syndrome coronavirus 2 (SARS-CoV-2) infection.

The present invention further provides, in yet another aspect, a method for preventing or treating an increase in the level of a cytokine in a patient in need, the method comprising administering to the patient a therapeutically-effective amount of the pharmaceutical composition described above.

In certain embodiments, the cytokine is selected from the group consisting of IL-6, IFN-Gamma, TNF-Alpha, and IL-1-Beta.

In certain embodiments, the increase in the level of the cytokine is associated with a Coronavirus infection or with a symptom thereof.

In certain embodiments, the increase in the level of the cytokine is measured in white blood cells (WBCs) of the patient, or in the serum of the patient.

The present invention further provides, in yet another aspect, a method for increasing the level of at least one stilbene selected from the group consisting of trans-piceid, cis-piceid, resveratrol, and ε-viniferin in a Vitis Vinifera cell optionally comprising at least one copy of an AroG* gene and optionally comprising at least one copy of a stilbene synthase (STS) gene, the method comprising contacting the cell with a composition comprising: (a) phenylalanine in a concentration of about 0.2 mM to about 5 mM, (b) p-coumaric acid in a concentration of about 0.1 mM to about 0.3 mM, or any combination of (a) and (b).

The present invention further provides, in yet another aspect, a method for increasing the level of at least one flavonoid selected from the group consisting of quercetin-3-glucoside, myricetin, catechin, epicatechin, epigallocatechin, procyanidin B1 and procyanidin B2 in a Vitis Vinifera cell optionally comprising at least one copy of an AroG* gene and optionally comprising at least one copy of a flavonol synthase (FLS) gene, the method comprising contacting the cell with a composition comprising: (a) phenylalanine in a concentration of about 0.2 mM to about 5 mM, (b) p-coumaric acid in a concentration of about 0.1 mM to about 0.3 mM, or, (c) any combination of (a) and (b).

The present invention further provides, in yet another aspect, an edible or a potable composition, comprising the pharmaceutical composition described above.

The present invention further provides, in yet another aspect, a pharmaceutical composition according described above, formulated for slow release or extended release.

Other aspects and features of the present disclosure will become apparent to those ordinarily skilled in the art upon review of the following description of specific embodiments in conjunction with the accompanying figures.

### BRIEF DESCRIPTION OF THE DRAWINGS

FIG. 1. Generation of transgenic Vitis Vinifera cv. Gamay cell culture expressing AroG* gene alone or both AroG* and STSs genes. (FIG. 1A) Schematic illustrations of transgene expression cassettes in pART27 vector. In gene constructs, transgene expression was under the control of 35S promoter. 35S, CaMV 35S promoter; Ω, TMV; HA: HA-tag sequence; AcV5, AcV5-tag sequence; T, OCS-terminator; KanR, Kanamycin resistance marker; LB, T-DNA left border; RB, T-DNA right border; AroG*, a feedback-insensitive bacterial form of 3-deoxy-D-arabinoheptulosonate 7-phosphate synthase enzyme (DAHPS); STS, stilbene synthase. (FIG. 1B) The expression of AroG* was monitored by immunoblot analysis with anti-HA antibody (1:500) antibody. The major protein band of AroG* at 35 and 45 kDa. (FIG. 1C) The expression of STS was monitored by immunoblot analysis with anti-AcV5 antibody (1: 1000) antibody. The major protein band of STS at 35 kDa. (FIG. 1D) The growth curves of the selected line which from each construction were measured by the increase in fresh weight from the day of reculturing (day 0) until beginning of cell death. (FIG. 1E) Cell morphology of each line on day 9 were taken by a Leica MZ FLII microscope.

FIG. 2. Principle component analysis plot of phenylpropanoid metabolites detected in control, AroG* and AroG*+STS samples. Values used for this analysis are normalized peak heights and then log-transformed data. Each cloud represent 8 replications of each line.

FIG. 3. Effect of co-expression of both AroG* and STS on the stilbenes biosynthesis precursors (FIG. 3A) and stilbenes (FIG. 3B) accumulation in Vitis transformated cells. Levels of metabolites are presented as fold change of transgenic lines in comparison with the control line (empty vector). Boxplot was a mixture of the metabolite content with each replication. In the control n = 24, including three lines, in the AroG* lines, n = 32, including four lines, while in the each AroG*+STS, n = 24, including three lines. Statistical significance was analysed by One-Way ANOVA, followed by Dunnett’s post hoc test. Black asterisk represents significant difference in metabolites content between control (empty) and transgenic lines, gray asterisk represent significant difference in metabolites content between single AroG* lines and double transgenic lines (p≤0.05; p≤0.01; p≤0.001).

FIG. 4. Effect of co-expression of both AroG and STS on the flavonoids accumulation in Vitis transformated cells. Levels of metabolites are presented as fold change of transgenic lines in comparison with the control line (empty vector). Boxplot was a mixture of the metabolite content with each replication. In the control n = 24, including 12 samples from two lines, in the AroG* lines, n = 32, including 8 samples from four lines, while in the each AroG*+STS, n = 24, including 8 samples from three lines. Statistical significance was analysed by One-Way ANOVA, followed by Dunnett’s post hoc test. Black asterisk represent significant difference in metabolites content between control (empty) and transgenic lines, gray asterisk represent significant difference in metabolites content between single AroG* lines and double transgenic lines (p≤0.05; p≤0.01; p≤0.001).

FIG. 5. Schematic representation of metabolic changes with fold change normalized to control (empty vector). The levels presented are the median value in each metabolite among different transgenic lines. Different transgenic lines were marked as pentagon (single AroG* line), diamond (AroG*+STS5), triangle (AroG*+STS10), hexagon (AroG*+STS28).

FIG. 6. Effect of different Phe and p-coumaric acid concentrations on growth and morphology. (FIG. 6A) Cell weight of 7-day old samples from different Phe concentrations treatments. (FIG. 6B) Cell weight of 7-day old samples from different p-coumaric acid concentrations treatments. (FIG. 6C) Microscopic photographs (taken by a Leica MZ FLII microscope) of AroG*+STS28 (Line 16) cells on day 7. The blue color staining is of dead cells which were stained with Evans blue.

FIG. 7. Effect of Phe and p-CA feeding of an AroG*+STS28 transformed line (line 16) on stilbenes production. Levels (mean ± SE, mg/g DW) of stilbenes are samples after 7 (black color) and 9 (gray color) days of treatment with different concentrations of Phe (FIG. 7A) and p-CA (FIG. 7B). Letters represent significant difference among samples using Two-Way ANOVA (P < 0.05) followed by a Tukey HSD post hoc test (P ≤ 0.05).

FIG. 8. Effect of Phe feeding of an AroG*+STS28 transformed line (line 16) on the production of precursors metabolites and flavonoids. Levels (mean ± SE) of metabolites, with 7 (black color) and 9 (gray color) days of treatment with different concentrations of Phe, are presented as peak heights. Letters represent significant difference among samples using Two-Way ANOVA (P < 0.05) followed by a Tukey HSD post hoc test (P ≤ 0.05).

FIG. 9. Effect of p-CA feeding of an AroG*+STS28 transformed line (line 16) on the production of precursors metabolites and flavonoids. Levels (mean ± SE) of metabolites, with 7 (black color) and 9 (gray color) days of treatment with different concentrations of Phe, are presented as peak heights. Letters represent significant difference among samples using Two-Way ANOVA (P < 0.05) followed by a Tukey HSD post hoc test (P ≤ 0.05). p-CA levels in different samples were not compared due to its exogenous.

FIG. 10. Schematic representation of changes in metabolites and gene expression levels between Phe fed cells and control which are collected after 9 days treatment. The levels are expressed as fold change which are normalized to control.

FIG. 11. Effect of Phe feeding of an AroG*+STS28 transformed line (Line 16) on CHS and STS gene expression. Levels (mean ± SE) of gene expression are presented as fold change which normalized to control samples. Stars represent significant differences in gene expression between control (black color) and 5 mM Phe fed samples (gray color) using student t-test (P < 0.05).

FIG. 12. Induction of anti-inflammatory gene expression in chicken WBCs at the mRNA level by LPS, and their inhibition by polyphenol extracts (GCE) according to the present invention.

FIG. 13. Inhibition of mRNA expression level of pro-inflammatory cytokines (INF-G, TNF, IL6) and induction of the anti-inflammatory cytokine mRNA (IL10), in spleens of chickens fed with grape-cell-powder (GCP) for 7 days and stimulated by LPS for 2 hours. Vertical lines indicate standard error (n = 10 in each group) *, P < 0.05 (t-test).

FIG. 14. Generation of transgenic V. vinifera cv. Gamay cell culture expressing AroG* and FLS genes. (FIG. 14A) Proposed flavonoid and stilbene biosynthetic pathways designed by co-expression of AroG with FLS in Vitis Vinifera cv. Gamay Red cell culture. (FIG. 14B) A schematic illustration of the transgene expression cassettes in the pART27 vectors. In the gene constructs, the transgene expression was under the control of the 35S promoter. 35S, CaMV 35S promoter; Ω, TMV; HA: HA-tag sequence; AcV5, AcV5-tag sequence; T, OCS-terminator; KanR, Kanamycin resistance marker; LB, T-DNA left border; RB, T-DNA right border; AroG*, a feedback-insensitive bacterial form of 3-deoxy-D-arabino-heptulosonate 7-phosphate synthase enzyme (DAHPS); FLS, flavonol synthase. (FIG. 14C) Accumulation of AroG* in the transgenic lines. Immunoblot analysis was performed using anti-HA antibody (1:500). The 35 kDa polypeptide represents AroG* protein. (FIG. 14D) Accumulation of FLS in the transgenic lines. Immunoblot analysis was performed using anti-AcV5 antibody (1:1000). The 35 kDa polypeptide represents the FLS protein. (FIG. 14E) Cell morphology of control (empty vector) and two AroG*+FLS lines on day 9 using a Leica MZ FLII microscope. The blue colored staining is of dead cells which were stained with Evans blue.

FIG. 15. Effect of co-expression of AroG* and FLS on Phe, Trp and p-CA accumulation in V. vinifera cv. Gamay Red transformed cells. Metabolite levels (mean ± SE, n=8) are presented as fold change of transgenic lines in comparison to control (empty vector). Statistical significance was analyzed by one-way ANOVA, followed by Dunnett’s post-hoc test. Black asterisks represent significant difference in metabolite content between control and transgenic lines and gray asterisks represent significant difference in metabolite content between AroG* lines and AroG*+FLS transgenic lines (* P < 0.05; ** P < 0.01; *** P < 0.001).

FIG. 16. Effect of co-expression of AroG* and FLS on stilbene accumulation in V. vinifera cv. Gamay Red transformed cells. Metabolite levels (mean ± SE, n=8) are presented as fold change of transgenic lines in comparison to the control (empty vector). Statistical significance was analyzed by one-way ANOVA, followed by Dunnett’s post-hoc test. Black asterisks represent significant difference in metabolite content between control and transgenic lines and gray asterisks represent significant difference in metabolite content between AroG* lines and AroG*+FLS transgenic lines (* P < 0.05; ** P < 0.01; *** P < 0.001).

FIG. 17. Effect of co-expression of AroG* and FLS on flavonols (a), flavan-3-ols (b) and anthocyanins (c) accumulation in V. vinifera cv. Gamay Red transformed cells. Metabolite levels (mean ± SE, n=8) are presented as fold change of transgenic lines in comparison to the control line (empty vector). Statistical significance was analyzed by one-way ANOVA, followed by Dunnett’s post-hoc test. Black asterisks represent significant difference in metabolite content between control and transgenic lines and gray asterisks represent significant difference in metabolite content between AroG* lines and AroG*+FLS transgenic lines (* P < 0.05; ** P < 0.01; *** P < 0.001).

FIG. 18. Summary of changes in metabolites and gene expression in V. vinifera cv. Gamay Red cells due to transformation of AroG* and FLS, and exogenous Phe feeding. Metabolites (FIG. 18A) and gene expression (FIG. 18B) levels are presented as fold changes in transgenic lines in comparison to non-fed control. Gene expression is visualized in a heatmap, and the differences are expressed by log2 fold change value which is centered and scaled for each row. Abbreviations: acet, acetyl; glu, glucoside; coum, coumaroyl; PAL, phenylalanine ammonia-lyase; C4H, cinnamate 4-hydroxylase; 4CL, 4-coumaroyl:CoA ligase; CHS, chalcone synthase; CHI, chalcone isomerase; F3H, flavanone 3-hydroxylase; F3′H and F3′5′H, flavonoid 3′ and 3′ 5′ hydroxylase; FLS, flavonol synthase; DFR, dihydroflavonol 4-reductase; LDOX, leucoanthocyanidin dioxygenase; LAR, leucoanthocyanidin reductase; ANR, anthocyanidin reductase; UFGT, uridine diphosphate-glucose: flavonoid 3-O-glucosyltransferase; OMT, O-methyltransferases; STS, Stilbene synthase.

### DETAILED DESCRIPTION

The present invention provides methods and compositions for increased production of flavonoids and stilbenes and in particular resveratrol.

According to the principles of the present invention, a combination of increased availability of Phe, with STS overexpression or FLS overexpression, diverts plant Phe metabolism into the stilbene pathway and/or the flavonoid pathway. The increase in Phe availability was achieved both by overexpressing AroG* and feeding the cell culture with external Phe. These attempts resulted in increased production of several resveratrol-derived stilbenes, in particular viniferin with a 600-fold increase in its concentration in comparison to non-treated cultures.

The present invention provides, in one aspect, a cell comprising at least one copy of an AroG* gene and at least one copy of a stilbene synthase (STS) gene.

In certain embodiments, the cell is a plant cell. In certain embodiments, the plant cell is a Vitis Vinifera cell. In certain embodiments, the Vitis Vinifera cell is a Vitis Vinifera cv. Gamay Red cell.

In certain embodiments, the AroG* gene is a plant gene. In certain embodiments, the AroG* gene is a Vitis Vinifera gene. In certain embodiments, the AroG* gene encodes a 3-Deoxy-D-arabinoheptulosonate 7-phosphate (DAHP) synthase (DAHPS) enzyme. In certain embodiments, the DAHPS enzyme catalyzes the chemical reaction: phosphoenolpyruvate + D-erythrose 4-phosphate + H2O ⇌ 3-deoxy-D-arabino-hept-2-ulosonate 7-phosphate + phosphate. In certain embodiments, the DAHPS enzyme is feedback-insensitive. In certain embodiments, the DAHPS enzyme is a phenylalanine-insensitive. In certain embodiments, the DAHPS enzyme is a tyrosine-insensitive. In certain embodiments, the DAHPS enzyme is a tryptophan-insensitive.

In certain embodiments, the DAHPS enzyme increases the availability of at least one amino-acid in the cell. In certain embodiments, the DAHPS enzyme increases the availability of at least one amino-acid selected from the group consisting of phenylalanine, tyrosine, and tryptophan in the cell. In certain embodiments, the DAHPS enzyme increases the availability of Phenylalanine in the cell. In certain embodiments, the DAHPS enzyme increases the availability of tyrosine in the cell. In certain embodiments, the DAHPS enzyme increases the availability of tryptophan in the cells

In certain embodiments, the STS gene is a plant gene. In certain embodiments, the STS gene is a Vitis Vinifera gene. In certain embodiments, the STS gene encodes an STS enzyme. In certain embodiments, the STS enzyme catalyzes the chemical reaction: 3 malonyl-CoA + 4-coumaroyl-CoA ⇌ 4 CoA + 3,4′,5-trihydroxy-stilbene (resveratrol) + 4 CO₂.

In certain embodiments, the STS enzyme increases the availability of at least one compound selected from the group consisting of dehydroepiandrosterone (DHEA), estrone, pregnenolone, and cholesterol in the cell. In certain embodiments, the STS enzyme increases the availability of DHEA in the cell. In certain embodiments, the STS enzyme increases the availability of estrone in the cell. In certain embodiments, the STS enzyme increases the availability of pregnenolone in the cell. In certain embodiments, the STS enzyme increases the availability of cholesterol in the cell. In certain embodiments, the STS enzyme increases the availability of resveratrol in the cell.

In certain embodiments, the STS enzyme produces a stilbene. In certain embodiments, the STS enzyme produces a stilbenoid. In certain embodiments, the stilbenoid is resveratrol. In certain embodiments, the stilbenoid is a resveratrol derivative. In certain embodiments, the resveratrol derivative is piceid.

In certain embodiments, the STS gene is a Vitis vinifera stilbene synthase (VvSTS) gene. In certain embodiments, the STS gene is selected from the group consisting of VvSTS5, VvSTS10 and VvSTS28. In certain embodiments, the STS gene is VvSTS5. In certain embodiments, the STS gene VvSTS10. In certain embodiments, the STS gene is VvSTS28.

The present invention provides, in one aspect, a cell comprising at least one copy of an AroG* gene and at least one copy of a flavonol synthase (FLS) gene.

In certain embodiments, the FLS gene is a plant gene. In certain embodiments, the FLS gene is a Vitis Vinifera gene. In certain embodiments, the FLS gene encodes an FLS enzyme. In certain embodiments, the FLS enzyme catalyzes the chemical reaction: 2-oxoglutarate + a (2R,3R)-dihydroflavonol + O₂ ⇌ a flavonol + CO₂ + H₂O + succinate.

In certain embodiments, the FLS enzyme produces a flavonoid. In certain embodiments, the FLS enzyme produces a flavonol. In certain embodiments, the flavonoid is flavonol. In certain embodiments, the flavonoid is flavan-3-ol. In certain embodiments, the flavonoid is anthocyanin. In certain embodiments, the flavonol is myricetin. In certain embodiments, the flavonol is quercetin-3-glucoside. In certain embodiments, the flavonol is kaempferol.

In certain embodiments, the FLS gene is a Vitis vinifera flavonol synthase (VvFLS) gene. In certain embodiments, the FLS gene is VIT_07s0031g00100.

The present invention provides, in one aspect, a doubly-transgenic Vitis Vinifera cell, comprising: at least one copy of an AroG* gene, and at least one copy of a stilbene synthase (STS) gene or a flavonol synthase (FLS) gene.

In certain embodiments, the AroG* gene or the STS gene is functionally-linked to a constitutive promoter. In certain embodiments, the AroG* gene and the STS gene are both functionally-linked to a constitutive promoter. In certain embodiments, the AroG* gene or the FLS gene is functionally-linked to a constitutive promoter. In certain embodiments, the AroG* gene, and the FLS gene are both functionally-linked to a constitutive promoter. In certain embodiments, the constitutive promoter is Cauliflower mosaic virus (CaMV) 35S RNA promoter (also known as “35S promoter”).

In certain embodiments, the AroG* gene and the STS gene are functionally-linked to different constitutive promoters. In certain embodiments, the AroG* gene and the STS gene are functionally-linked to the same constitutive promoter. In certain embodiments, the AroG* gene and the STS gene are functionally-linked to the same constitutive promoter found upstream to the AroG* gene which is found upstream to the STS gene. In certain embodiments, the AroG* gene and the STS gene are functionally-linked to the same constitutive promoter found upstream to the STS gene which is found upstream to the AroG*gene.

In certain embodiments, the AroG* gene and the FLS gene are functionally-linked to different constitutive promoters. In certain embodiments, the AroG* gene and the FLS gene are functionally-linked to the same constitutive promoter. In certain embodiments, the AroG* gene and the FLS gene are functionally-linked to the same constitutive promoter found upstream to the AroG* gene which is found upstream to the FLS gene. In certain embodiments, the AroG* gene and the FLS gene are functionally-linked to the same constitutive promoter found upstream to the FLS gene which is found upstream to the AroG*gene.

In certain embodiments, the cell described herein comprises a higher level of at least one stilbenoid. In certain embodiments, the stilbenoid comprises resveratrol. In certain embodiments, the stilbenoid comprises trans-piceid. In certain embodiments, the stilbenoid comprises cis-piceid. In certain embodiments, the stilbenoid comprises ε-viniferin.

In certain embodiments, the cell described herein comprises a higher level of at least one flavonoid. In certain embodiments, the flavonoid comprises flavonols. In certain embodiments, the flavonoid comprises flavan-3-ol. In certain embodiments, the flavonoid comprises anthocyanins. In certain embodiments, the flavonol comprises myricetin, quercetin-3-glucoside, or kaempferol. In certain embodiments, the flavonol comprises myricetin. In certain embodiments, the flavonol comprises quercetin-3-glucoside. In certain embodiments, the flavonol comprises kaempferol. In certain embodiments, the flavan-3-ol comprises catechin, epicatechin, epigallocatechin, procyanidin B1 or procyanidin B2. In certain embodiments, the anthocyanin comprises cyanidin-3 -glucoside, cyanidin-3 -acetyl-glucoside, cyanidin-3 -coumaroyl-glucoside, peonidin-3-glucoside, peonidin-3-acetyl-glucoside, peonidin-3-coumaroyl-glucoside, delphindin-3 -glucoside, delphindin-3 -acetyl-glucoside, delphindin-3 -coumaroyl-glucoside, malvidin-3-glucoside, malvidin-3-acetyl-glucoside, malvidin-3-coumaroyl-glucoside, petunidin-3-glucoside, petunidin-3-acetyl-glucoside, or petunidin-3-coum-glu.

In certain embodiments, the cell described above comprises a higher level of: at least one amino-acid selected from the group consisting of Phenylalanine, Tryptophan, and p-CA (p-coumaric acid); at least one stilbene selected from the group consisting of trans-piceid, cis-piceid, resveratrol, and ε-viniferin; at least one flavonoid selected from the group consisting of quercetin-3-glucoside, myricetin, and procyanidin B2; at least one anthocyanin selected from the group consisting of malvidin-3-o-glucoside, malvidin-3-com-glucoside, malvidin-3-acetyl-glucoside, and petunidin-3-com-glucoside; or any combination of the above, compared to a corresponding non-transgenic Vitis Vinifera cell or compared to a corresponding singly-transgenic Vitis Vinifera cell.

In certain embodiments, the cell described herein comprises a higher level of: at least one amino-acid selected from the group consisting of Phenylalanine, Tryptophan, and p-CA (p-coumaric acid); at least one stilbene selected from the group consisting of trans-piceid, cis-piceid, resveratrol, and ε-viniferin; at least one flavonoid selected from the group consisting of quercetin-3-glucoside, myricetin, catechin, epicatechin, epigallocatechin, procyanidin B1 and procyanidin B2; at least one anthocyanin selected from the group consisting of cyanidin, peonidin, delphinidin, petunidin, and malvidin; or any combination of the above, compared to a corresponding non-transgenic Vitis Vinifera cell or compared to a corresponding singly-transgenic Vitis Vinifera cell.

In certain embodiments, the cell described above comprises a higher level of at least one amino-acid selected from the group consisting of Phenylalanine, Tryptophan, and p-CA (p-coumaric acid) compared to a corresponding non-transgenic Vitis Vinifera cell.

In certain embodiments, the cell described above comprises a higher level of at least one stilbene selected from the group consisting of trans-piceid, cis-piceid, resveratrol, and ε-viniferin, compared to a corresponding non-transgenic Vitis Vinifera cell.

In certain embodiments, the cell described above comprises a higher level of at least one flavonoid selected from the group consisting of quercetin-3-glucoside, myricetin, catechin, epicatechin, epigallocatechin, procyanidin B1 and procyanidin B2, compared to a corresponding non-transgenic Vitis Vinifera cell.

In certain embodiments, the cell described above comprises a higher level of at least one anthocyanin selected from the group consisting of malvidin-3-o-glucoside, malvidin-3-com-glucoside, malvidin-3-acetyl-glucoside, and petunidin-3-com-glucoside, compared to a corresponding non-transgenic Vitis Vinifera cell.

In certain embodiments, the cell described above comprises a higher level of at least one anthocyanin selected from the group consisting of cyanidin, peonidin, delphinidin, petunidin, and malvidin, compared to a corresponding non-transgenic Vitis Vinifera cell.

In certain embodiments, the cell described above comprises a higher level of at least one amino-acid selected from the group consisting of Phenylalanine, Tryptophan, and p-CA (p-coumaric acid) compared to a corresponding singly-transgenic Vitis Vinifera cell.

In certain embodiments, the cell described above comprises a higher level of at least one stilbene selected from the group consisting of trans-piceid, cis-piceid, resveratrol, and ε-viniferin, compared to a corresponding singly-transgenic Vitis Vinifera cell.

In certain embodiments, the cell described above comprises a higher level of at least one flavonoid selected from the group consisting of quercetin-3-glucoside, myricetin, catechin, epicatechin, epigallocatechin, procyanidin B1 and procyanidin B2, compared to a corresponding singly-transgenic Vitis Vinifera cell.

In certain embodiments, the cell described above comprises a higher level of at least one anthocyanin selected from the group consisting of malvidin-3-o-glucoside, malvidin-3-com-glucoside, malvidin-3-acetyl-glucoside, and petunidin-3-com-glucoside, compared to a corresponding singly-transgenic Vitis Vinifera cell.

In certain embodiments, the cell described above comprises a higher level of at least one anthocyanin selected from the group consisting of cyanidin, peonidin, delphinidin, petunidin, and malvidin, compared to a corresponding singly-transgenic Vitis Vinifera cell.

In certain embodiments, the corresponding singly-transgenic Vitis Vinifera cell comprises at least one copy of the AroG* gene. In certain embodiments, the corresponding singly-transgenic Vitis Vinifera cell comprises the same number of copies of the AroG* gene.

In certain embodiments, the cell described above comprises a concentration of at least 0.3 mg/g dry weight trans-piceid, a concentration of at least 0.3 mg/g dry weight cis-piceid, a concentration of at least 0.1 mg/g dry weight resveratrol, a concentration of at least 0.02 mg/g dry weight ε-viniferin, or any combination of the above.

In certain embodiments, the cell described above comprises a concentration of at least 0.3 mg/g dry weight trans-piceid. In certain embodiments, the cell described above comprises a concentration of at least 0.3 mg/g dry weight cis-piceid. In certain embodiments, the cell described above comprises a concentration of at least 0.1 mg/g dry weight resveratrol. In certain embodiments, the cell described above comprises a concentration of at least 0.02 mg/g dry weight ε-viniferin.

In certain embodiments, the cell described above comprises a concentration of at least 0.3 mg/g dry weight trans-piceid, a concentration of at least 0.3 mg/g dry weight cis-piceid, a concentration of at least 0.1 mg/g dry weight resveratrol, and a concentration of at least 0.02 mg/g dry weight s-viniferin.

In certain embodiments, the cell described above comprises a concentration of at least 0.5 mg/g dry weight trans-piceid, a concentration of at least 0.5 mg/g dry weight cis-piceid, a concentration of at least 0.8 mg/g dry weight resveratrol, a concentration of at least 0.6 mg/g dry weight ε-viniferin, or any combination of the above.

In certain embodiments, the cell described above comprises a concentration of at least 0.5 mg/g dry weight trans-piceid. In certain embodiments, the cell described above comprises a concentration of at least 0.6 mg/g dry weight trans-piceid. In certain embodiments, the cell described above comprises a concentration of at least 0.7 mg/g dry weight trans-piceid. In certain embodiments, the cell described above comprises a concentration of at least 0.8 mg/g dry weight trans-piceid. In certain embodiments, the cell described above comprises a concentration of at least 0.9 mg/g dry weight trans-piceid. In certain embodiments, the cell described above comprises a concentration of at least 1.0 mg/g dry weight trans-piceid. In certain embodiments, the cell described above comprises a concentration of at least 1.1 mg/g dry weight trans-piceid. In certain embodiments, the cell described above comprises a concentration of at least 1.2 mg/g dry weight trans-piceid.

In certain embodiments, the cell described above comprises a concentration of at least 0.5 mg/g dry weight cis-piceid. In certain embodiments, the cell described above comprises a concentration of at least 0.6 mg/g dry weight cis-piceid. In certain embodiments, the cell described above comprises a concentration of at least 0.7 mg/g dry weight cis-piceid. In certain embodiments, the cell described above comprises a concentration of at least 0.8 mg/g dry weight cis-piceid. In certain embodiments, the cell described above comprises a concentration of at least 0.9 mg/g dry weight cis-piceid. In certain embodiments, the cell described above comprises a concentration of at least 1.0 mg/g dry weight cis-piceid. In certain embodiments, the cell described above comprises a concentration of at least 1.1 mg/g dry weight cis-piceid. In certain embodiments, the cell described above comprises a concentration of at least 1.2 mg/g dry weight cis-piceid.

In certain embodiments, the cell described above comprises a concentration of at least 0.5 mg/g dry weight resveratrol. In certain embodiments, the cell described above comprises a concentration of at least 0.6 mg/g dry weight resveratrol. In certain embodiments, the cell described above comprises a concentration of at least 0.7 mg/g dry weight resveratrol. In certain embodiments, the cell described above comprises a concentration of at least 0.8 mg/g dry weight resveratrol. In certain embodiments, the cell described above comprises a concentration of at least 0.9 mg/g dry weight resveratrol. In certain embodiments, the cell described above comprises a concentration of at least 1.0 mg/g dry weight resveratrol. In certain embodiments, the cell described above comprises a concentration of at least 1.1 mg/g dry weight resveratrol. In certain embodiments, the cell described above comprises a concentration of at least 1.2 mg/g dry weight resveratrol. In certain embodiments, the cell described above comprises a concentration of at least 1.3 mg/g dry weight resveratrol.

In certain embodiments, the cell described above comprises a concentration of at least 0.5 mg/g dry weight ε-viniferin. In certain embodiments, the cell described above comprises a concentration of at least 0.6 mg/g dry weight ε-viniferin. In certain embodiments, the cell described above comprises a concentration of at least 0.7 mg/g dry weight ε-viniferin. In certain embodiments, the cell described above comprises a concentration of at least 0.8 mg/g dry weight ε-viniferin. In certain embodiments, the cell described above comprises a concentration of at least 0.9 mg/g dry weight ε-viniferin. In certain embodiments, the cell described above comprises a concentration of at least 1.0 mg/g dry weight ε-viniferin. In certain embodiments, the cell described above comprises a concentration of at least 1.1 mg/g dry weight ε-viniferin. In certain embodiments, the cell described above comprises a concentration of at least 1.2 mg/g dry weight s-viniferin.

In certain embodiments, the cell described above comprises a concentration of at least 0.5 mg/g dry weight myricetin. In certain embodiments, the cell described above comprises a concentration of at least 0.6 mg/g dry weight myricetin. In certain embodiments, the cell described above comprises a concentration of at least 0.7 mg/g dry weight myricetin. In certain embodiments, the cell described above comprises a concentration of at least 0.8 mg/g dry weight myricetin. In certain embodiments, the cell described above comprises a concentration of at least 0.9 mg/g dry weight myricetin. In certain embodiments, the cell described above comprises a concentration of at least 1.0 mg/g dry weight myricetin. In certain embodiments, the cell described above comprises a concentration of at least 1.1 mg/g dry weight myricetin. In certain embodiments, the cell described above comprises a concentration of at least 1.2 mg/g dry weight myricetin.

In certain embodiments, the cell described above comprises a concentration of at least 0.5 mg/g dry weight quercetin-3-glucoside. In certain embodiments, the cell described above comprises a concentration of at least 0.6 mg/g dry weight quercetin-3-glucoside. In certain embodiments, the cell described above comprises a concentration of at least 0.7 mg/g dry weight quercetin-3-glucoside. In certain embodiments, the cell described above comprises a concentration of at least 0.8 mg/g dry weight quercetin-3-glucoside. In certain embodiments, the cell described above comprises a concentration of at least 0.9 mg/g dry weight quercetin-3-glucoside. In certain embodiments, the cell described above comprises a concentration of at least 1.0 mg/g dry weight quercetin-3-glucoside. In certain embodiments, the cell described above comprises a concentration of at least 1.1 mg/g dry weight quercetin-3-glucoside. In certain embodiments, the cell described above comprises a concentration of at least 1.2 mg/g dry weight quercetin-3-glucoside.

In certain embodiments, the cell described above comprises a concentration of at least 0.5 mg/g dry weight catechin. In certain embodiments, the cell described above comprises a concentration of at least 0.6 mg/g dry weight catechin. In certain embodiments, the cell described above comprises a concentration of at least 0.7 mg/g dry weight catechin. In certain embodiments, the cell described above comprises a concentration of at least 0.8 mg/g dry weight catechin. In certain embodiments, the cell described above comprises a concentration of at least 0.9 mg/g dry weight catechin. In certain embodiments, the cell described above comprises a concentration of at least 1.0 mg/g dry weight catechin. In certain embodiments, the cell described above comprises a concentration of at least 1.1 mg/g dry weight catechin. In certain embodiments, the cell described above comprises a concentration of at least 1.2 mg/g dry weight catechin.

In certain embodiments, the cell described above comprises a concentration of at least 0.5 mg/g dry weight epicatechin. In certain embodiments, the cell described above comprises a concentration of at least 0.6 mg/g dry weight epicatechin. In certain embodiments, the cell described above comprises a concentration of at least 0.7 mg/g dry weight epicatechin. In certain embodiments, the cell described above comprises a concentration of at least 0.8 mg/g dry weight epicatechin. In certain embodiments, the cell described above comprises a concentration of at least 0.9 mg/g dry weight epicatechin. In certain embodiments, the cell described above comprises a concentration of at least 1.0 mg/g dry weight epicatechin. In certain embodiments, the cell described above comprises a concentration of at least 1.1 mg/g dry weight epicatechin. In certain embodiments, the cell described above comprises a concentration of at least 1.2 mg/g dry weight epicatechin.

In certain embodiments, the cell described above comprises a concentration of at least 0.5 mg/g dry weight epigallocatechin. In certain embodiments, the cell described above comprises a concentration of at least 0.6 mg/g dry weight epigallocatechin. In certain embodiments, the cell described above comprises a concentration of at least 0.7 mg/g dry weight epigallocatechin. In certain embodiments, the cell described above comprises a concentration of at least 0.8 mg/g dry weight epigallocatechin. In certain embodiments, the cell described above comprises a concentration of at least 0.9 mg/g dry weight epigallocatechin. In certain embodiments, the cell described above comprises a concentration of at least 1.0 mg/g dry weight epigallocatechin. In certain embodiments, the cell described above comprises a concentration of at least 1.1 mg/g dry weight epigallocatechin. In certain embodiments, the cell described above comprises a concentration of at least 1.2 mg/g dry weight epigallocatechin.

In certain embodiments, the cell described above comprises a concentration of at least 0.5 mg/g dry weight procyanidin B1. In certain embodiments, the cell described above comprises a concentration of at least 0.6 mg/g dry weight procyanidin B1. In certain embodiments, the cell described above comprises a concentration of at least 0.7 mg/g dry weight procyanidin B1. In certain embodiments, the cell described above comprises a concentration of at least 0.8 mg/g dry weight procyanidin B1. In certain embodiments, the cell described above comprises a concentration of at least 0.9 mg/g dry weight procyanidin B1. In certain embodiments, the cell described above comprises a concentration of at least 1.0 mg/g dry weight procyanidin B1. In certain embodiments, the cell described above comprises a concentration of at least 1.1 mg/g dry weight procyanidin B1. In certain embodiments, the cell described above comprises a concentration of at least 1.2 mg/g dry weight procyanidin B1.

In certain embodiments, the cell described above comprises a concentration of at least 0.5 mg/g dry weight procyanidin B2. In certain embodiments, the cell described above comprises a concentration of at least 0.6 mg/g dry weight procyanidin B2. In certain embodiments, the cell described above comprises a concentration of at least 0.7 mg/g dry weight procyanidin B2. In certain embodiments, the cell described above comprises a concentration of at least 0.8 mg/g dry weight procyanidin B2. In certain embodiments, the cell described above comprises a concentration of at least 0.9 mg/g dry weight procyanidin B2. In certain embodiments, the cell described above comprises a concentration of at least 1.0 mg/g dry weight procyanidin B2. In certain embodiments, the cell described above comprises a concentration of at least 1.1 mg/g dry weight procyanidin B2. In certain embodiments, the cell described above comprises a concentration of at least 1.2 mg/g dry weight procyanidin B2.

In certain embodiments, the cell described above comprises a similar or lower level of: at least one flavonoid selected from the group consisting of myricetin, quercetin-3-glucoside, catechin, epicatechin, epigallocatechin, and procyanidin; at least one anthocyanin selected from the group consisting of cyanidin, peonidin, delphinidin, petunidin, and malvidin; or any combination of the above, compared to a corresponding non-transgenic Vitis Vinifera cell or compared to a corresponding singly-transgenic Vitis Vinifera cell.

In certain embodiments, the cell described above comprises a similar or lower level of at least one flavonoid selected from the group consisting of myricetin, quercetin-3-glucoside, catechin, epicatechin, epigallocatechin, and procyanidin, compared to a corresponding non-transgenic Vitis Vinifera cell or compared to a corresponding singly-transgenic Vitis Vinifera cell.

In certain embodiments, the cell described above comprises a similar or lower level of at least one anthocyanin selected from the group consisting of cyanidin, peonidin, delphinidin, petunidin, and malvidin, compared to a corresponding non-transgenic Vitis Vinifera cell or compared to a corresponding singly-transgenic Vitis Vinifera cell.

In certain embodiments, the corresponding singly-transgenic Vitis Vinifera cell comprises at least one copy of the AroG* gene. In certain embodiments, the corresponding singly-transgenic Vitis Vinifera cell comprises the same number of copies of the AroG* gene.

In certain embodiments, the cell described above comprises resveratrol in a concentration of about 1.26 mg/g dry weight, ε-viniferin in a concentration of about 10.8 mg/g dry weight, or both. In certain embodiments, the cell described above comprises resveratrol in a concentration of about 1.26 mg/g dry weight. In certain embodiments, the cell described above comprises ε-viniferin in a concentration of about 10.8 mg/g dry weight.

The present invention further provides, in another aspect, a method for maintaining a cell optionally comprising at least one copy of an AroG * gene, optionally comprising at least one copy of a stilbene synthase (STS) gene, and optionally comprising at least one copy of a flavonol synthase (FLS) gene, the method comprising contacting the cell with a composition comprising: phenylalanine in a concentration of about 0.2 mM to about 5 mM, p-coumaric acid in a concentration of about 0.1 mM to about 0.3 mM, or any combination of the above.

The present invention further provides, in another aspect, a method for maintaining a cell optionally comprising at least one copy of an AroG* gene and optionally comprising at least one copy of a stilbene synthase (STS) gene, the method comprising contacting the cell with a composition comprising: phenylalanine in a concentration of about 0.2 mM to about 5 mM, p-coumaric acid in a concentration of about 0.1 mM to about 0.3 mM, or any combination of the above.

The present invention further provides, in another aspect, a method for maintaining a cell optionally comprising at least one copy of an AroG* gene and optionally comprising at least one copy of a flavonol synthase (FLS) gene, the method comprising contacting the cell with a composition comprising: phenylalanine in a concentration of about 0.2 mM to about 5 mM, p-coumaric acid in a concentration of about 0.1 mM to about 0.3 mM, or any combination of the above.

In certain embodiments, the cell is a transgenic cell. In certain embodiments, the cell is a plant cell. In certain embodiments, the cell is a transgenic plant cell. In certain embodiments, the plant cell is a Vitis Vinifera cell. In certain embodiments, the cell comprises at least one copy of an AroG* gene. In certain embodiments, the cell comprises at least one copy of an STS gene. In certain embodiments, the cell comprises at least one copy of an FLS gene. In certain embodiments, the cell comprises a single copy of an AroG* gene. In certain embodiments, the cell comprises a single copy of an STS gene. In certain embodiments, the cell comprises a single copy of an FLS gene.

In certain embodiments, the method described above comprises contacting the cell with a composition comprising phenylalanine in a concentration of at least about 0.2 mM. In certain embodiments, the method described above comprises contacting the cell with a composition comprising phenylalanine in a concentration of at least about 0.5 mM. In certain embodiments, the method described above comprises contacting the cell with a composition comprising phenylalanine in a concentration of at least about 1.0 mM. In certain embodiments, the method described above comprises contacting the cell with a composition comprising phenylalanine in a concentration of at least about 2.0 mM. In certain embodiments, the method described above comprises contacting the cell with a composition comprising phenylalanine in a concentration of at least about 5.0 mM.

In certain embodiments, the method described above comprises contacting the cell with a composition comprising phenylalanine in a concentration of about 0.2 mM to about 5 mM. In certain embodiments, the method described above comprises contacting the cell with a composition comprising phenylalanine in a concentration of about 0.5 mM to about 5 mM. In certain embodiments, the method described above comprises contacting the cell with a composition comprising phenylalanine in a concentration of about 1 mM to about 5 mM. In certain embodiments, the method described above comprises contacting the cell with a composition comprising phenylalanine in a concentration of about 2 mM to about 5 mM.

In certain embodiments, the method described above comprises contacting the cell with a composition comprising phenylalanine in a concentration of about 0.2 mM. In certain embodiments, the method described above comprises contacting the cell with a composition comprising phenylalanine in a concentration of about 0.5 mM. In certain embodiments, the method described above comprises contacting the cell with a composition comprising phenylalanine in a concentration of about 1 mM. In certain embodiments, the method described above comprises contacting the cell with a composition comprising phenylalanine in a concentration of about 2 mM.In certain embodiments, the method described above comprises contacting the cell with a composition comprising phenylalanine in a concentration of about 5 mM.

In certain embodiments, the method described above comprises contacting the cell with a composition comprising p-coumaric acid in a concentration of at least about 0.1 mM. In certain embodiments, the method described above comprises contacting the cell with a composition comprising p-coumaric acid in a concentration of at least about 0.3 mM.

In certain embodiments, the method described above comprises contacting the cell with a composition comprising p-coumaric acid in a concentration of about 0.1 to about 0.3 mM.

In certain embodiments, the method described above comprises contacting the cell with a composition comprising p-coumaric acid in a concentration of about 0.1 mM. In certain embodiments, the method described above comprises contacting the cell with a composition comprising p-coumaric acid in a concentration of about 0.3 mM.

The present invention further provides, in yet another aspect, a pharmaceutical composition, comprising a doubly-transgenic Vitis Vinifera cell as described above, or an extract or fraction thereof.

In certain embodiments, the doubly-transgenic Vitis Vinifera cell was maintained by the method described above.

The present invention further provides, in yet another aspect, a pharmaceutical composition, comprising a non-transgenic Vitis Vinifera cell or a single-transgenic Vitis Vinifera cell comprising at least one copy of an AroG* gene, wherein the cell was maintained by the method described above, or an extract or fraction thereof.

In certain embodiments, the pharmaceutical composition described above comprises: at least one amino-acid selected from the group consisting of Phenylalanine, Tryptophan, and p-CA; at least one stilbene selected from the group consisting of trans-piceid, cis-piceid, resveratrol, and ε-viniferin; at least one flavonoid selected from the group consisting of quercetin-3-glucoside, and procyanidin B2; at least one anthocyanin selected from the group consisting of malvidin-3-o-glucoside, malvidin-3-com-glucoside, malvidin-3-acetyl-glucoside, petunidin-3-com-glucoside, procyanidin B2, and myricetin; or any combination of the above.

In certain embodiments, the pharmaceutical composition described above comprises: at least one amino-acid selected from the group consisting of Phenylalanine, Tryptophan, and p-CA; at least one stilbene selected from the group consisting of trans-piceid, cis-piceid, resveratrol, and ε-viniferin; at least one flavonoid selected from the group consisting of quercetin-3-glucoside, myricetin, myricetin, catechin, epicatechin, epigallocatechin, procyanidin B1 and procyanidin B2; at least one anthocyanin selected from the group consisting of cyanidin, peonidin, delphinidin, petunidin, and malvidin; or any combination of the above.

In certain embodiments, the pharmaceutical composition described above comprises at least one amino-acid selected from the group consisting of Phenylalanine, Tryptophan, and p-CA. In certain embodiments, the pharmaceutical composition described above comprises at least one stilbene selected from the group consisting of trans-piceid, cis-piceid, resveratrol, and ε-viniferin. In certain embodiments, the pharmaceutical composition described above comprises at least one flavonoid selected from the group consisting of quercetin-3-glucoside, and procyanidin B2. In certain embodiments, the pharmaceutical composition described above comprises at least one flavonoid selected from the group consisting of quercetin-3-glucoside, myricetin, myricetin, catechin, epicatechin, epigallocatechin, procyanidin B1 and procyanidin B2. In certain embodiments, the pharmaceutical composition described above comprises at least one anthocyanin selected from the group consisting of malvidin-3-o-glucoside, malvidin-3-com-glucoside, malvidin-3-acetyl-glucoside, and petunidin-3-com-glucoside. In certain embodiments, the pharmaceutical composition described above comprises at least one anthocyanin selected from the group consisting of cyanidin, peonidin, delphinidin, petunidin, and malvidin.

In certain embodiments, the pharmaceutical composition described above comprises an extract of the doubly-transgenic Vitis Vinifera cell or the single-transgenic Vitis Vinifera cell. In certain embodiments, the pharmaceutical composition described above comprises an extract of the doubly-transgenic Vitis Vinifera cell. In certain embodiments, the pharmaceutical composition described above comprises an extract of the single-transgenic Vitis Vinifera cell.

In certain embodiments, the extract is a polyphenol extract. In certain embodiments, the extract is in the form of a dried powder. In certain embodiments, the extract is a grape cell polyphenol extract (GCE). In certain embodiments, the extract is in the form of a dried grape cell powder (GCP).

In certain embodiments, the fraction is a polyphenol fraction. In certain embodiments, the fraction is in the form of a dried powder. In certain embodiments, the fraction is a grape cell polyphenol fraction (GCF). In certain embodiments, the fraction is in the form of a dried grape cell powder.

In certain embodiments, the pharmaceutical composition described above comprises a cytoplasmic fraction of the doubly-transgenic Vitis Vinifera cell or the single-transgenic Vitis Vinifera cell. In certain embodiments, the pharmaceutical composition described above comprises a cytoplasmic fraction of the doubly-transgenic Vitis Vinifera cell. In certain embodiments, the pharmaceutical composition described above comprises a cytoplasmic fraction of the single-transgenic Vitis Vinifera cell.

In certain embodiments, the pharmaceutical composition described above comprises a polyphenolic fraction of the doubly-transgenic Vitis Vinifera cell or the single-transgenic Vitis Vinifera cell. In certain embodiments, the pharmaceutical composition described above comprises a polyphenolic fraction of the doubly-transgenic Vitis Vinifera cell. In certain embodiments, the pharmaceutical composition described above comprises a polyphenolic fraction of the single-transgenic Vitis Vinifera cell.

In certain embodiments, the pharmaceutical composition described above comprises the vacuole of the doubly-transgenic Vitis Vinifera cell or the single-transgenic Vitis Vinifera cell. In certain embodiments, the pharmaceutical composition described above comprises the vacuole of the doubly-transgenic Vitis Vinifera cell. In certain embodiments, the pharmaceutical composition described above comprises the vacuole of the single-transgenic Vitis Vinifera cell.

In certain embodiments, the pharmaceutical composition described above is substantially dehydrated composition. In certain embodiments, the pharmaceutical composition described above comprises 0% to 50% by weight water. In certain embodiments, the pharmaceutical composition described above comprises 0% to 40% by weight water. In certain embodiments, the pharmaceutical composition described above comprises 0% to 30% by weight water. In certain embodiments, the pharmaceutical composition described above comprises 0% to 20% by weight water. In certain embodiments, the pharmaceutical composition described above comprises 0% to 10% by weight water. In certain embodiments, the pharmaceutical composition described above comprises 0% to 5% by weight water. In certain embodiments, the pharmaceutical composition described above comprises 0% to 1% by weight water.

In certain embodiments, the pharmaceutical composition described above is substantially devoid of intact cells. In certain embodiments, the pharmaceutical composition described above comprises 0% to 50% by weight intact cells. In certain embodiments, the pharmaceutical composition described above comprises 0% to 40% by weight intact cells. In certain embodiments, the pharmaceutical composition described above comprises 0% to 30% by weight intact cells. In certain embodiments, the pharmaceutical composition described above comprises 0% to 20% by weight intact cells. In certain embodiments, the pharmaceutical composition described above comprises 0% to 10% by weight intact cells. In certain embodiments, the pharmaceutical composition described above comprises 0% to 5% by weight intact cells. In certain embodiments, the pharmaceutical composition described above comprises 0% to 1% by weight intact cells.

In certain embodiments, the pharmaceutical composition described above is substantially devoid of ruptured cells. In certain embodiments, the pharmaceutical composition described above comprises 0% to 50% by weight ruptured cells. In certain embodiments, the pharmaceutical composition described above comprises 0% to 40% by weight ruptured cells. In certain embodiments, the pharmaceutical composition described above comprises 0% to 30% by weight ruptured cells. In certain embodiments, the pharmaceutical composition described above comprises 0% to 20% by weight ruptured cells. In certain embodiments, the pharmaceutical composition described above comprises 0% to 10% by weight ruptured cells. In certain embodiments, the pharmaceutical composition described above comprises 0% to 5% by weight ruptured cells. In certain embodiments, the pharmaceutical composition described above comprises 0% to 1% by weight ruptured cells.

In certain embodiments, disclosed herein are methods of preventing, treating, reducing the incidence, suppressing or inhibiting a viral infection, disease, disorder or symptom thereof in a subject.

In certain embodiments, disclosed herein are methods of preventing, treating, reducing the incidence, suppressing or inhibiting a viral infection, disease, disorder or symptom thereof in a subject, comprising the step of administering to the subject a pharmaceutical composition as described herein in detail.

In certain embodiments, the term “viral disease” may encompass a pathological condition caused either directly or indirectly from the presence of a virus in a subject. The term “viral disease” may further encompass a clinical manifestation or symptom resulting from or associated with infection of a virus, that includes without limitation, a viral disease caused by Severe acute respiratory syndrome coronavirus 2 (SARS-CoV-2).

In certain embodiments, the terms “virus” and “viral” may encompass a disease-causing agent that includes Coronavirus (CoV), Severe acute respiratory syndrome (SARS) virus, Middle East respiratory syndrome (MERS) virus and Influenza virus infection.

In certain embodiments, a method of preventing, treating, reducing the incidence, suppressing or inhibiting a viral infection, disease, disorder or symptom thereof in a subject comprises reducing cytokine release syndrome (CRS) or cytokine storm in the subject. In certain embodiments, the method comprises reducing cytokine release syndrome (CRS) in the subject. In certain embodiments, the method comprises reducing cytokine storm in the subject.

In certain embodiments, the viral infection comprises Coronavirus (CoV) infection, a Severe acute respiratory syndrome (SARS) infection, a Middle East respiratory syndrome (MERS) infection, or an Influenza virus infection.

In certain embodiments, the Coronavirus infection comprises Severe acute respiratory syndrome coronavirus 2 (SARS-CoV-2) infection. In certain embodiments, a viral infection comprises Severe acute respiratory syndrome coronavirus 2 (SARS-CoV-2) infection.

In one embodiment, the term “SARS—CoV—2”, also known as “2019 novel coronavirus (2019-nCoV)”, “severe acute respiratory syndrome-related coronavirus (SARSr-CoV)”, “Wuhan coronavirus”, “Wuhan virus”, “Chinese virus”, “COVID-19 virus” or “coronavirus” is a positive-sense single-stranded RNA (+ssRNA) virus belonging to the Coronaviridae family of viruses, known as coronaviruses. SARS—CoV—2 was first identified in December 2019 in Wuhan, China. In certain embodiments, SARS—CoV—2 is transmitted through human-to-human transmission, generally via respiratory droplets as sneeze, cough or exhalation. A skilled artisan will recognize that SARS-CoV-2 is a member of the subgenus Sarbecovirus, having an RNA sequence of approximately 30,000 bases in length. The present disclosure comprises methods for treating all coronavirus variants. A skilled artisan will recognize that seven coronaviruses are known to infect humans. In certain embodiments, coronavirus comprises Human coronavirus 229E (HCoV-229E). In certain embodiments, coronavirus comprises Human coronavirus OC43 (HCoV—OC43). In certain embodiments, coronavirus comprises Severe acute respiratory syndrome-related coronavirus (SARS—CoV). In certain embodiments, coronavirus comprises Human coronavirus NL63 (HCoV—NL63, New Haven coronavirus). In certain embodiments, coronavirus comprises Human coronavirus HKU1. In certain embodiments, coronavirus comprises Middle East respiratory syndrome-related coronavirus (MERS—CoV), previously known as novel coronavirus 2012 and HCoV-EMC. In certain embodiments, coronavirus comprises SARS—CoV—2.

In certain embodiments, the disease comprises coronavirus disease-2019 (COVID-19).

In certain embodiments, SARS—CoV—2 viral infection causes a respiratory illness, termed “coronavirus disease 2019” (COVID-19), also known as “novel coronavirus pneumonia (NCP)”, “SARS—CoV—2 acute respiratory disease”, and “2019-nCoV acute respiratory disease”. In certain embodiments, COVID-19 symptoms appear after an incubation period of between 2 to 14 days. In certain embodiments, coronavirus primarily affects the lower respiratory tract. In certain embodiments, coronavirus primarily affects the upper respiratory tract. In certain embodiments, COVID-19 symptoms comprise fever, coughing, shortness of breath, pain in the muscles, tiredness, pneumonia, acute respiratory distress syndrome, sepsis, septic shock, death, or any combination thereof.

In certain embodiments, a viral disease is caused by SARS—CoV—2. In certain embodiments, a viral disease is caused by Coronavirus. In certain embodiments, a viral disease is caused by SARS virus. In certain embodiments, a viral disease is caused by MERS virus. In certain embodiments, a viral disease is caused by Influenza virus.

In certain embodiments, “cytokine release syndrome” or “CRS” may encompass systemic inflammatory response syndrome (SIRS) or cytokine storm syndromes (CSS), that can be triggered by a variety of factors such as infections and certain drugs. In certain embodiments, CRS comprises activation of white blood cells which release inflammatory cytokines. In certain embodiments, CRS comprises elevated levels of various cytokines, such as MCP-1, IL-8, IL-6, TNF-α, IFN—γ, and IL-10. A skilled artisan would appreciate that “cytokine storm” may encompass an immediate-onset CRS. In certain embodiments, CRS or cytokine storm can occur as a result of an infectious or non-infectious disease, including coronavirus disease 2019 (COVID-19).

In one embodiment, the term “elevated” may encompass increased amount or level, for example, an “elevated cytokine level” may refer to a cytokine level that is higher than the cytokine level measured in a blood sample of a healthy individual.

In certain embodiments, the pharmaceutical compositions disclosed herein are used to treat or prevent a viral infection. In certain embodiments, the pharmaceutical compositions disclosed herein are used to treat or prevent Coronavirus. In certain embodiments, the pharmaceutical compositions disclosed herein are used to treat or prevent SARS. In certain embodiments, the pharmaceutical compositions disclosed herein are used to treat or prevent MERS. In certain embodiments, the pharmaceutical compositions disclosed herein are used to treat or prevent Influenza.

In certain embodiments, the disclosure provides methods of preventing or treating a viral disease, for example COVID-2019 in a subject, comprising administering any of the compositions disclosed herein.

In certain embodiments, a virus comprises a Coronavirus (CoV), a Severe acute respiratory syndrome (SARS), a Middle East respiratory syndrome (MERS), an Influenza virus, or mutations thereof. In certain embodiments, a Coronavirus comprises Severe acute respiratory syndrome coronavirus 2 (SARS-CoV-2). In certain embodiments, a virus comprises Severe acute respiratory syndrome coronavirus 2 (SARS-CoV-2).

The present invention further provides, in yet another aspect, a method for treating a Coronavirus infection or a symptom thereof in a patient in need, the method comprising administering to the patient a therapeutically-effective amount of a pharmaceutical composition as described above. In certain embodiments, disclosed herein are methods of preventing, treating, reducing the incidence, suppressing or inhibiting a Coronavirus infection or a symptom thereof in a patient in need, the method comprising administering to the patient a therapeutically-effective amount of the pharmaceutical composition.In certain embodiments, the symptom is a cytokine storm. In certain embodiments, the symptom comprises elevated cytokine levels. In another embodiment, the symptom comprises elevated IL-6 levels. In certain embodiments, the symptom comprises elevated IL-8 levels. In another embodiment, the symptom comprises elevated IL-17A levels.

In certain embodiments, disclosed herein are methods of treating, preventing, ameliorating, inhibiting, or reducing the incidence of a cytokine release syndrome (CRS) or a cytokine storm in a subject. In certain embodiments, methods of treating, preventing, ameliorating, inhibiting, or reducing the incidence of a cytokine release syndrome (CRS) or a cytokine storm, due to a viral infection, disease or disorder in a subject, comprise administering to the subject a pharmaceutical composition as described herein in detail.

In certain embodiments, methods of treating, preventing, ameliorating, inhibiting, or reducing the incidence of a cytokine release syndrome (CRS), a cytokine storm, further comprises the step of administering to the subject one or more additional compositions comprising therapeutic agents or anti-viral agents.

In certain embodiments, the pharmaceutical composition is systemically administered to the patient. In certain embodiments, the pharmaceutical composition is orally administered to the patient. In certain embodiments, the pharmaceutical composition is formulated for oral administration.

The present invention further provides, in yet another aspect, a crop plant or part thereof, comprising at least one copy of an AroG* gene.

The present invention further provides, in yet another aspect, a crop plant or part thereof, comprising at least one copy of an AroG* gene and at least one copy of a stilbene synthase (STS) gene.

The present invention further provides, in yet another aspect, a crop plant or part thereof, comprising at least one copy of an AroG* gene and at least one copy of a stilbene synthase (STS) gene or a flavonol synthase (FLS) gene.

The present invention further provides, in yet another aspect, a crop plant or part thereof, comprising at least one copy of an AroG* gene and at least one copy of a flavonol synthase (FLS) gene.

In certain embodiments, the part of the crop plant is not an isolated cell.

In certain embodiments, the crop plant is Vitis Vinifera.

In certain embodiments, the Vitis Vinifera is Gamay Red cultivar.

The present invention further provides, in yet another aspect, a method for preventing or treating a Cytokine Storm in a patient in need, the method comprising administering to the patient a therapeutically-effective amount of the pharmaceutical composition described above.

The present invention further provides, in yet another aspect, a method for preventing or treating of treating, preventing, ameliorating, inhibiting, or reducing the incidence of a Cytokine Release Syndrome (CRS) or a Cytokine Storm in a patient in need, the method comprising administering to the patient a therapeutically-effective amount of the pharmaceutical composition described above.

In certain embodiments, the methods of the present invention are prophylactic, and are for prevention. In certain embodiments, the methods of the present invention are therapeutic, and are for treatment.

In certain embodiments, the CRS or Cytokine Storm is associated with a Coronavirus infection or with a symptom thereof. In certain embodiments, the CRS or Cytokine Storm is associated with a Coronavirus infection. In certain embodiments, the CRS or Cytokine Storm is associated with a symptom of Coronavirus infection. In certain embodiments, the Coronavirus infection comprises Severe acute respiratory syndrome coronavirus 2 (SARS-CoV-2) infection.

The present invention further provides, in yet another aspect, a method for preventing or treating an increase in the level of a cytokine in a patient in need, the method comprising administering to the patient a therapeutically-effective amount of the pharmaceutical composition described above.

In certain embodiments, the level of the cytokine is measured in blood or serum. In certain embodiments, the level of the cytokine is measured in whole blood. In certain embodiments, the level of the cytokine is measured in serum. In certain embodiments, the level of the cytokine is measured in blood cells.

In certain embodiments, the cytokine is selected from the group consisting of IL-6, IFN-Gamma, TNF-Alpha, and IL-1-Beta. In certain embodiments, the cytokine is IL-6. In certain embodiments, the cytokine is IFN-Gamma. In certain embodiments, the cytokine is TNF-Alpha. In certain embodiments, the cytokine is IL-1-Beta.

In certain embodiments, the present invention provides methods of decreasing the production of inflammatory cytokines. In certain embodiments, the inflammatory cytokine comprises IL-6. In certain embodiments, the inflammatory cytokine comprises IFN-Gamma. In certain embodiments, the inflammatory cytokine comprises TNF-Alpha. In certain embodiments, the inflammatory cytokine comprises IL-1-Beta.

In certain embodiments, the increase in the level of the cytokine is associated with a Coronavirus infection or with a symptom thereof. In certain embodiments, the increase in the level of the cytokine is associated with a Coronavirus infection. In certain embodiments, the increase in the level of the cytokine is associated with a symptom of Coronavirus infection. In certain embodiments, the Coronavirus infection comprises Severe acute respiratory syndrome coronavirus 2 (SARS-CoV-2) infection.

In certain embodiments, the increase in the level of the cytokine is measured in white blood cells (WBCs) of the patient, or in the serum of the patient. In certain embodiments, the increase in the level of the cytokine is measured in WBCs of the patient. In certain embodiments, the increase in the level of the cytokine is measured in the serum of the patient.

The present invention further provides, in yet another aspect, a method for increasing the level of at least one stilbene selected from the group consisting of trans-piceid, cis-piceid, resveratrol, and ε-viniferin in a Vitis Vinifera cell optionally comprising at least one copy of an AroG* gene and optionally comprising at least one copy of a stilbene synthase (STS) gene, the method comprising contacting the cell with a composition comprising: (a) phenylalanine in a concentration of about 0.2 mM to about 5 mM, (b) p-coumaric acid in a concentration of about 0.1 mM to about 0.3 mM, or, (c) any combination of (a) and (b).

In certain embodiments, the at least one stilbene is trans-piceid. In certain embodiments, the at least one stilbene is cis-piceid. In certain embodiments, the at least one stilbene is resveratrol. In certain embodiments, the at least one stilbene is ε-viniferin.

The present invention further provides, in yet another aspect, a method for increasing the level of at least one flavonoid selected from the group consisting of quercetin-3-glucoside, myricetin, catechin, epicatechin, epigallocatechin, procyanidin B1 and procyanidin B2 in a Vitis Vinifera cell optionally comprising at least one copy of an AroG* gene and optionally comprising at least one copy of a flavonol synthase (FLS) gene, the method comprising contacting the cell with a composition comprising: (a) phenylalanine in a concentration of about 0.2 mM to about 5 mM, (b) p-coumaric acid in a concentration of about 0.1 mM to about 0.3 mM, or, (c) any combination of (a) and (b).

In certain embodiments, the at least one flavonoid is quercetin-3-glucoside. In certain embodiments, the at least one flavonoid is myricetin. In certain embodiments, the at least one flavonoid is catechin. In certain embodiments, the at least one flavonoid is epicatechin. In certain embodiments, the at least one flavonoid is epigallocatechin. In certain embodiments, the at least one flavonoid is procyanidin B1. In certain embodiments, the at least one flavonoid is procyanidin B2.

In certain embodiments, the Vitis Vinifera cell optionally comprises at least one copy of an AroG* gene and optionally comprises at least one copy of a stilbene synthase (STS) gene. In certain embodiments, the Vitis Vinifera cell comprises at least one copy of an AroG* gene and optionally comprises at least one copy of a stilbene synthase (STS) gene. In certain embodiments, the Vitis Vinifera cell optionally comprises at least one copy of an AroG* gene and comprises at least one copy of a stilbene synthase (STS) gene. In certain embodiments, the Vitis Vinifera cell comprises at least one copy of an AroG* gene and comprises at least one copy of a stilbene synthase (STS) gene.

In certain embodiments, the Vitis Vinifera cell optionally comprises at least one copy of an AroG* gene and optionally comprises at least one copy of a flavonol synthase (FLS) gene. In certain embodiments, the Vitis Vinifera cell comprises at least one copy of an AroG* gene and optionally comprises at least one copy of a flavonol synthase (FLS) gene. In certain embodiments, the Vitis Vinifera cell optionally comprises at least one copy of an AroG* gene and comprises at least one copy of a flavonol synthase (FLS) gene. In certain embodiments, the Vitis Vinifera cell comprises at least one copy of an AroG* gene and comprises at least one copy of a flavonol synthase (FLS) gene.

The present invention further provides, in yet another aspect, an edible or a potable composition, comprising the pharmaceutical composition described above.

The present invention further provides, in yet another aspect, a pharmaceutical composition described above, formulated for slow release or extended release.

In certain embodiments, the pharmaceutical composition is formulated for slow release. In certain embodiments, the pharmaceutical composition is formulated for extended release.

### Definitions

As used herein, the singular form “a”, “an” and “the” include plural references unless the context clearly dictates otherwise. For example, the term “a molecule” also includes a plurality of molecules. As used herein the term “about” may encompass a deviance of between 0.0001-5% from the indicated number or range of numbers. In one embodiment, the term “about”, may encompass a deviance of between 1 -10% from the indicated number or range of numbers. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

The terms “subject,” “individual,” and “patient” are used interchangeably herein, and refer to human or non-human animals to whom treatment with a composition or formulation in accordance with the present invention is provided.

The above-described embodiments are intended to be examples only. Alterations, modifications and variations can be affected to the particular embodiments by those of skill in the art. The scope of the claims should not be limited by the particular embodiments set forth herein but should be construed in a manner consistent with the specification as a whole.

### EXAMPLES

The following Materials and methods are used in the Examples:

### Plant Material and Cell Growth

V. vinifera cv. Gamay Red cell suspensions were established from young grape berries as described previously (Kiselev et al., 2013). The wild type (wt) cells were maintained under solid B5 medium supplemented with 250 mg/L casein hydrolysate, 100 mg/L myo-inositol, 0.2 mg/L kinetin and 0.1 mg/L NAA, 2% sucrose (w/v).11,27 Liquid cell suspension cultures were prepared with the same composition of nutrients and hormones and maintained at 25 ± 1° C. with continuous gentle shaking under constant light conditions (25 µmol m-2 s-1). A 50 mL stock of suspended cells was maintained by sub-culturing 5 g of cells to a fresh medium once a week.

### Plasmid Generation and Preparation of Stably Transformed Cell Lines

The binary vector construct for ectopic expression of AroG* and FLS or STS were prepared by using the vectors and cloning system described previously (Wang et al., 2021). The full-length FLS cDNA of VIT_07s0031g00100 (NCBI: XM_002283156) or STS cDNAs of VvSTS5, VvSTS10 and VvSTS28 were cloned into pGEM®-T Easy Vector Systems (Promega, Madison, WI, USA).

The ORFs of FLS and STS were cloned into pART7 vector under the control of the double cauliflower mosaic virus (CaMV) 35S promoter and was fused with C-terminal AcV5 tag. The AroG* gene was PCR amplified and assembled with the above FLS and STS gene cassettes into an empty pART27 vector using Gibson Assembly cloning system (Gibson et al., 2009). The construct contains a kanamycin selection marker. Agrobacterium tumefaciens EHA105 were transformed with these constructs using the freeze-thaw method. Agrobacterium-mediated transformation was performed as described previously (Wang et al., 2021).

### Immunoblot Analysis

Immunoblots were performed using the following antibodies: monoclonal anti-HA antibody (sc-7392; 1:500 dilution; Santa Cruz Biotechnology, Inc. Dallas, Texas, USA) to detected AroG* protein, and mouse monoclonal anti-Acv5 antibody (sc65499; 1:500 dilutions; Santa Cruz Biotechnology, Inc. Dallas, Texas, USA) for the FLS and STS proteins.

### Metabolites Extraction and Profiling by LC-MS

Grape cells were collected on day 9 and washed three times with cold dH2O. After lyophilization, 40 mg cells were extracted for metabolites according to the method described in ref 40. Separation and identification of metabolites were carried out by using ultra-performance liquid chromatography coupled to a quadrupole time-of-flight mass-spectrometer (UPLC-QTOF-MS, Waters, MA, USA). Analytical standards: resveratrol and piceid were obtained from Sigma-Aldrich (St. Louis, MO, USA) and ε-viniferin was obtained from Extrasynthese (ZI Lyon Nord, Genay Cedex, France).

### Phe Feeding

Feeding experiment with 5 mM Phe (Merck Darmstadt, Germany) was carried out in triplicate, 4 h after subculturing as described previously (Wang et al., 2021). After 9 days, samples were collected for LC-MS and gene expression analysis.

### Real-Time PCR

Total RNA was isolated from the grape cells by ZR Plant RNA MiniprepTM (Zymo Research, Irvine, USA) followed by DNase treatment (Qiagen, Valencia, CA, USA). First-strand cDNA was synthesized from 2 µg RNA using RevertAid reverse transcriptase (Thermo Fisher Scientific Inc, Waltham, MA, USA), and RT-qPCR was performed according to the protocol described in Wang et al., 2021. Relative expression was determined by normalizing the values of the reference gene of ubiquitin and β-Actin. PAL, CHS, F3′H, F3′5H, DFR members identified based on the grapevine PN 40024 12X V2 coverage.

### Statistical Analysis

LC-MS data were normalized to internal standards and sample weight. A comparison between the metabolic profiles of control and transgenic samples or between AroG* line and AroG* + FLS lines or AroG* + STS lines were carried out by one-way ANOVA followed by Dunnett’s test. The differences in gene expression before and after feeding for different lines were analyzed by two-way ANOVA followed Tukey’s HSD test. All the statistical analyses were performed using the JMP 14.0 (SAS Institute, Inc, Cary, NC). Gene expression level was showed by log2 fold change value, which was centered and scaled by row and was visualized in the heatmap using “ggplot2” package (R v4.0.1 in RStudio).

### Example 1. Generation of Transgenic Vitis Vinifera Cv. Gamay Red Cell Culture OverExpressing AroG* and AroG* + STS Genes

Previous studies revealed that over-expression of AroG* in the Gamay Red cell culture, resulted in elevated levels of Phe and its derived metabolites, including resveratrol (Manela et al., 2015). This suggests that Phe availability is a limiting factor in production of resveratrol. To further increase the levels of stilbenes, Gamay Red cell cultures were transformed with both (a) AroG*, a feedback-insensitive bacterial form of 3-Deoxy-D-arabinoheptulosonate 7-phosphate (DAHP) synthase (DAHPS), for increasing the availability of amino-acids and in particular Phe, and (b) STS, for directing the carbon flow towards the production of stilbene.

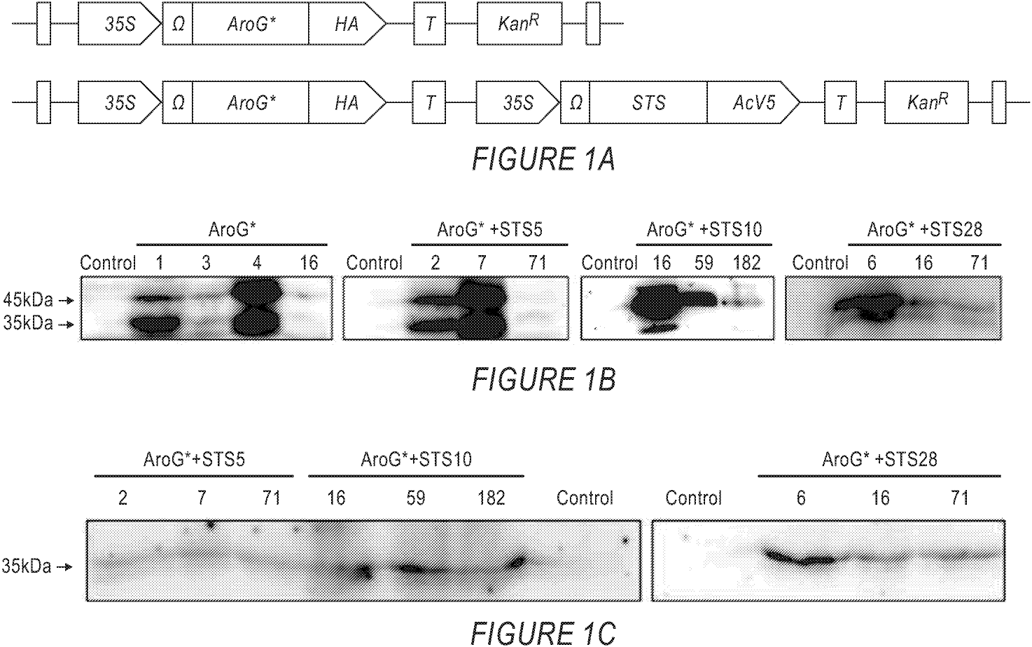

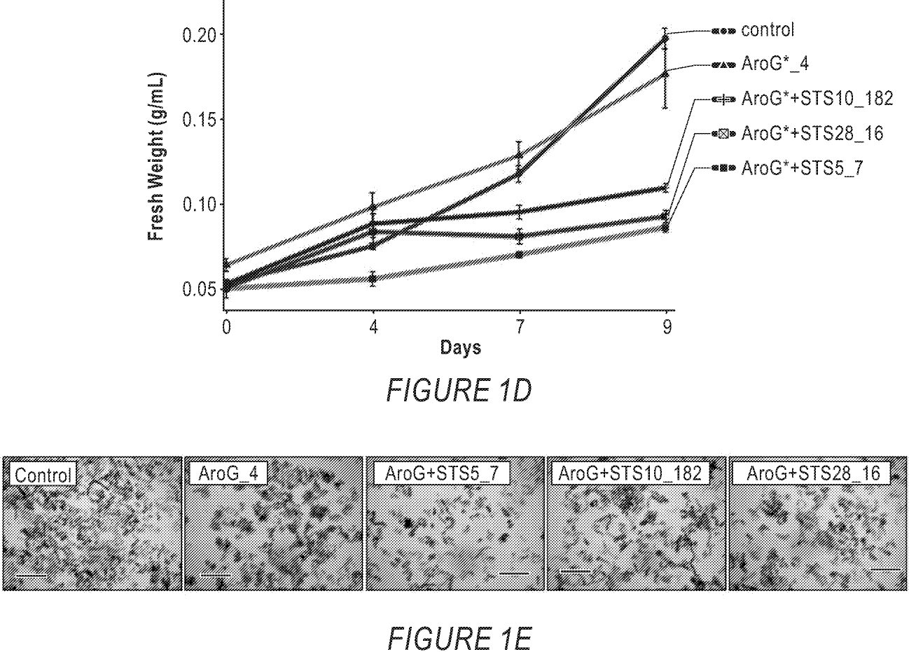

Among 48 Vitis vinifera stilbene synthase (VvSTS) genes in grape, VvSTS5, VvSTS10 and VvSTS28 were selected and cloned, based on the results that piceid levels following transient expression of these three STS genes were higher than that of other selected STS genes in N. benthamiana leaves. (Parage et al., 2012). Four constructs were designed, including AroG*, AroG* + STS5, AroG* + STS10, AroG* + STS28 genes, all expressed under control of the 35S promoter (FIG. 1A).

These four constructs were expressed in Vitis Vinifera cv. Gamay Red Cell Culture using Agrobacterium tumefaciens-mediated transformation. Controls are wild type cell lines transformed with an empty vector containing only the kanamycin resistance cassette. Independent transformed lines of each construct were chosen based on their Kanamycin resistance and accumulation of AroG* HA-tagged protein (FIG. 1B) and STS AcV5-tagged proteins, with a certain variation in protein accumulation between the lines (FIG. 1C).

AroG* protein accumulation in the transgenic lines, revealed the two polypeptides, ~35 and 45 kDa (FIG. 1B) in agreement with the predicted size of the mature polypeptide and the un-cleaved protein, respectively similar to previous reports (Tzin, Oliva, Manela).

Transgenic cultures were grown in liquid media for three weeks before experiments in order to increase growth rate and maintain a homogenous environment for the cells.

Four AroG* lines and 3 lines of each of the double constructs (AroG* and STS5, 10 or 28) were selected for metabolic analysis based on their stable vigor. The growth rate of the AroG* + STSs lines in liquid media, determined by the increase in fresh weight of the culture, was about 1.8 times slower than controls and single AroG* lines (FIG. 1D). The visual phenotype of the transgenic cells were almost identical to those of controls (FIG. 1E).

### Example 2. Effect of Co-Expression of AroG* and STS Genes on the Metabolic Profile of Grape Cell Culture

A metabolomics comparison was performed between control, AroG* and AroG* + STStransgenic lines. Samples from day 9 (as in FIG. 1D) were subjected to ultraperformance liquid chromatography-tandem mass spectrometry (UPLC-MS) analysis to detect metabolites that differentially accumulate in the transgenic lines.

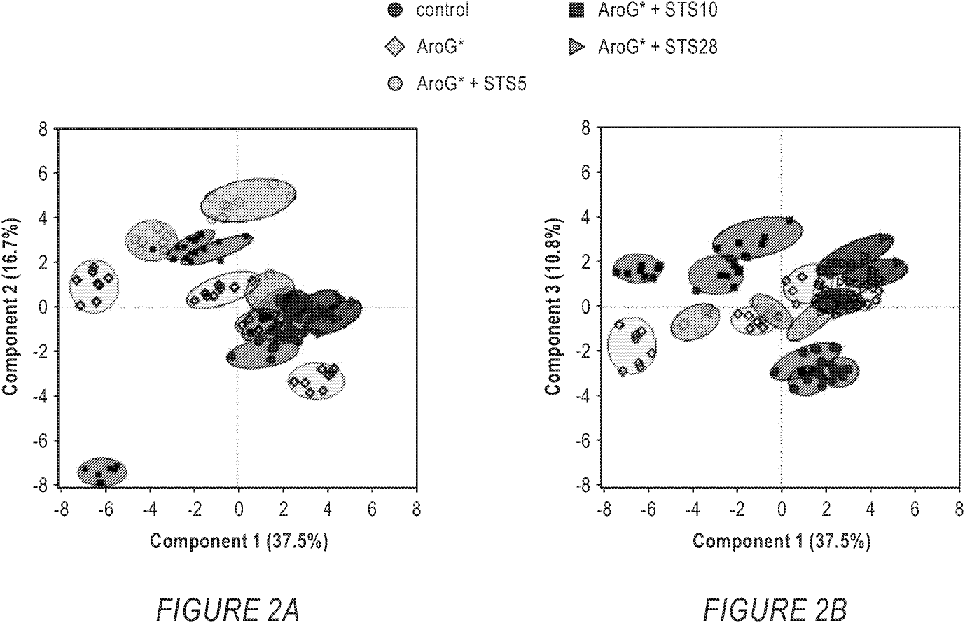

Principal component analysis (PCA) were performed on the annotated metabolites data (FIG. 2A). Variations in the metabolite profile generated a distinct separation between control and transgenic lines on the PCA plot (FIG. 2B). Differences in the metabolite data between AroG* and AroG* + STS were minor. The feature of PCA suggest that shikimate-derived metabolites and stilbenes contribute greatly to the difference between control and transgenic lines (Table 1).

[symbol]

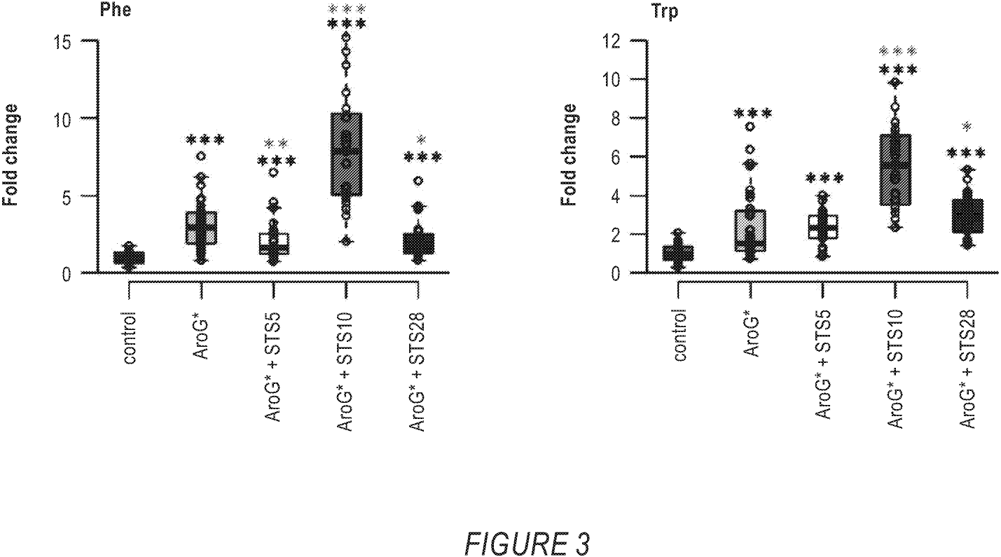

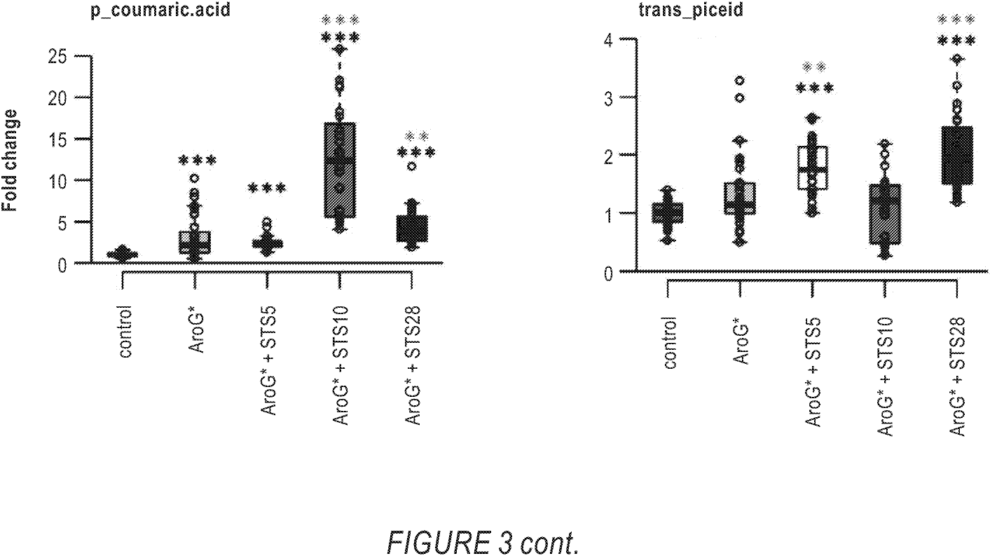

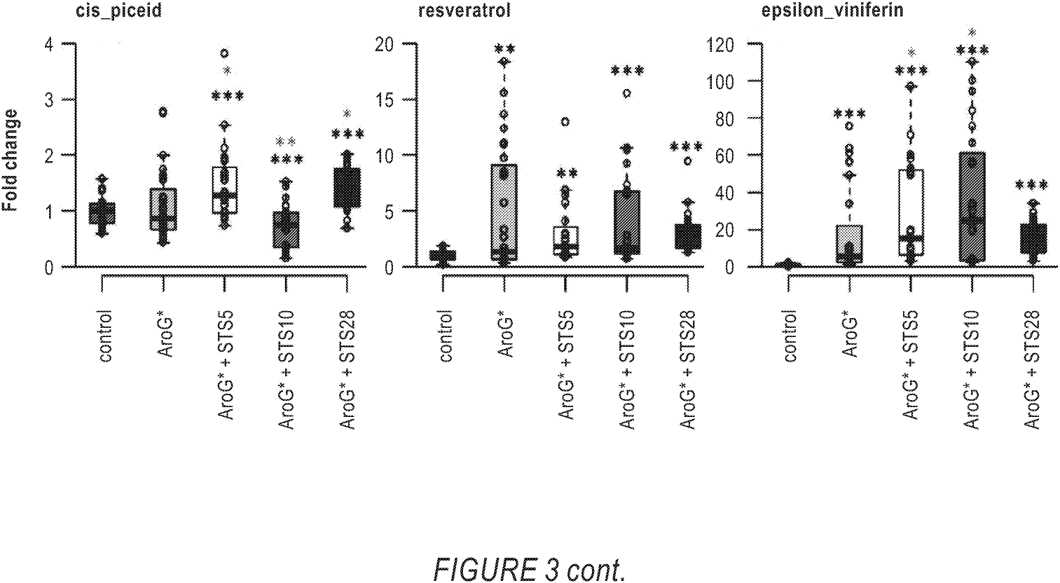

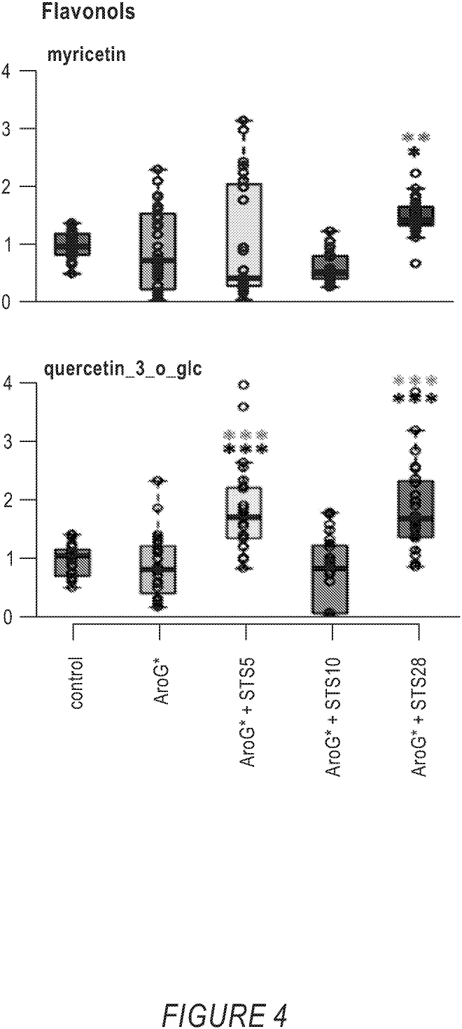

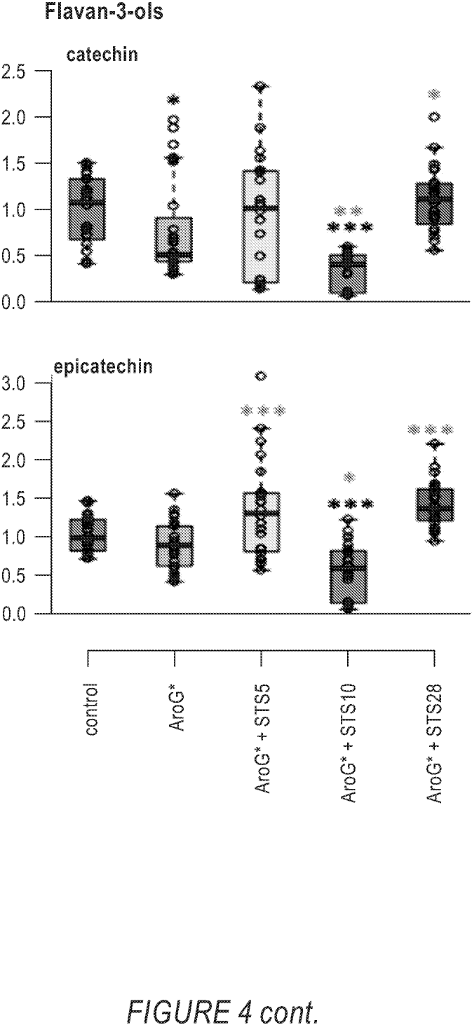

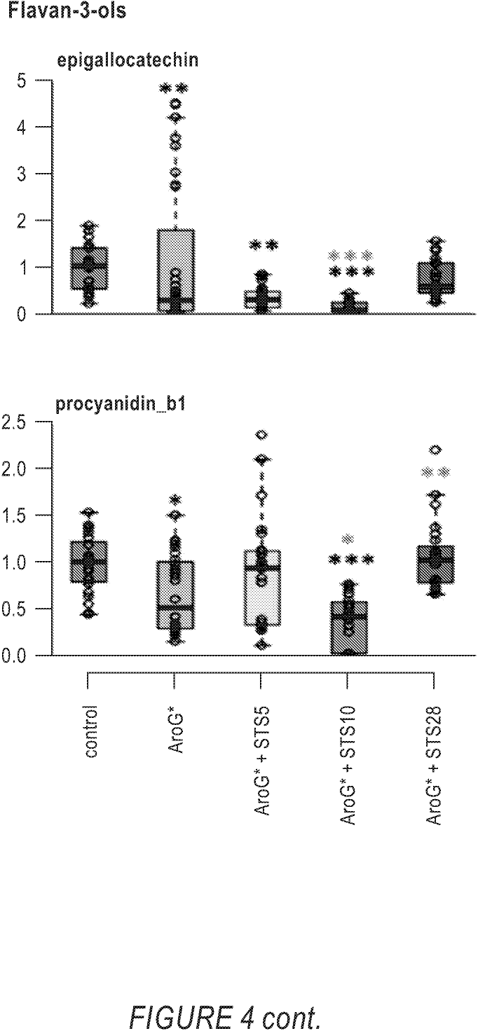

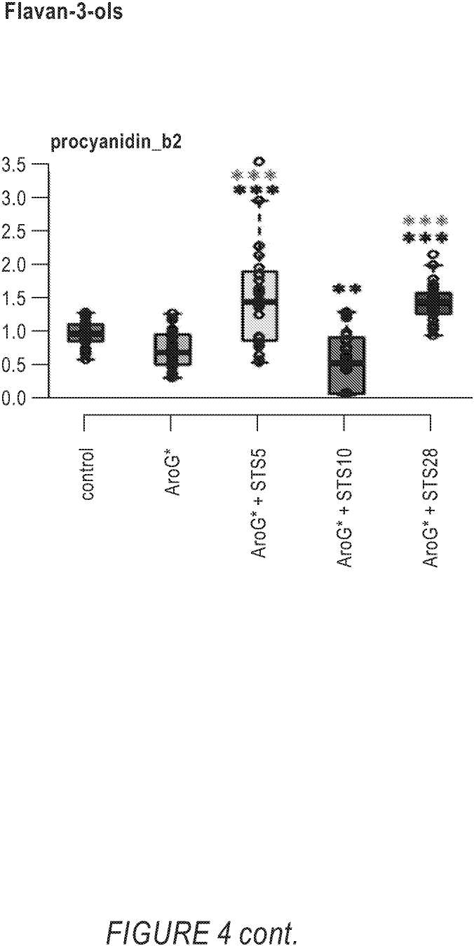

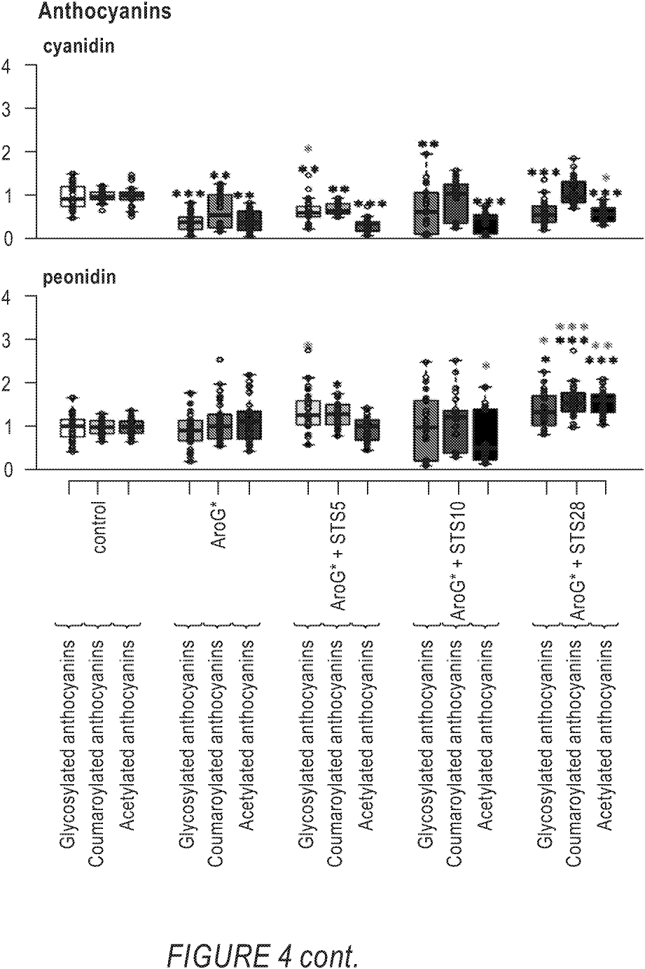

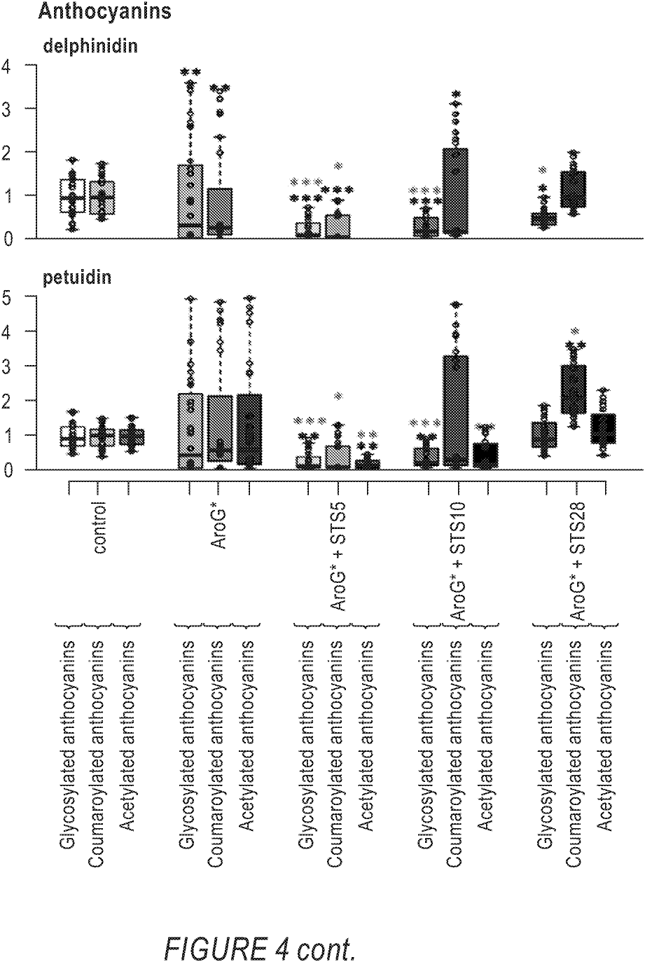

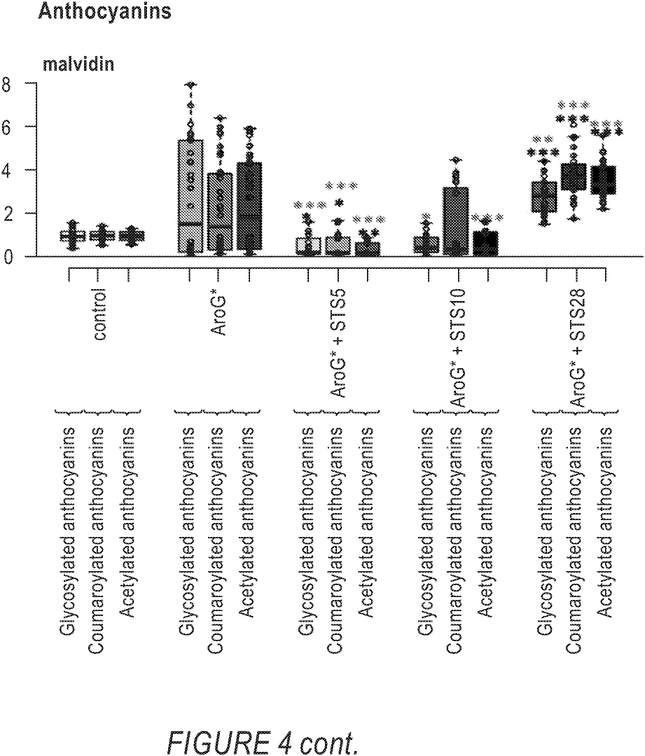

To obtain a global view of the effect of expression of the AroG* and STS transgenes on grape metabolism, two comparisons analyses of metabolites were performed on the data: (i) a comparison between the control and the transgenic lines and (ii) a comparison between the AroG* alone and the double transgenic lines, here all lines from same construct as a group/pool (FIG. 3 and FIG. 4).

All transgenic lines had significantly higher levels of two of the three aromatic amino acids derived from the shikimate pathway, Phe, Tryptophan (Trp) and of Phe-derived p-CA (p-coumaric acid), a common precursor for both the stilbene and flavonoid pathways (FIG. 3, top panel). These results are consistent with previous findings in Arabidopsis, tomato, petunia and grape cell cultures (Manela et al., 2015; Oliva et al., 2015; Tzin et al., 2012; Tzin et al., 2015) in which AroG* overexpression resulted in increased production of amino-acids and phenolic acids.

In the double transgenic lines (AroG* + STS) Phe levels were significantly higher with STS10 gene in comparison to AroG* alone, and Trp and p-CA levels increased significantly in the double transgenic lines AroG* + STS10 and AroG* + STS28 in comparison to AroG*. (FIG. 3, top panel).

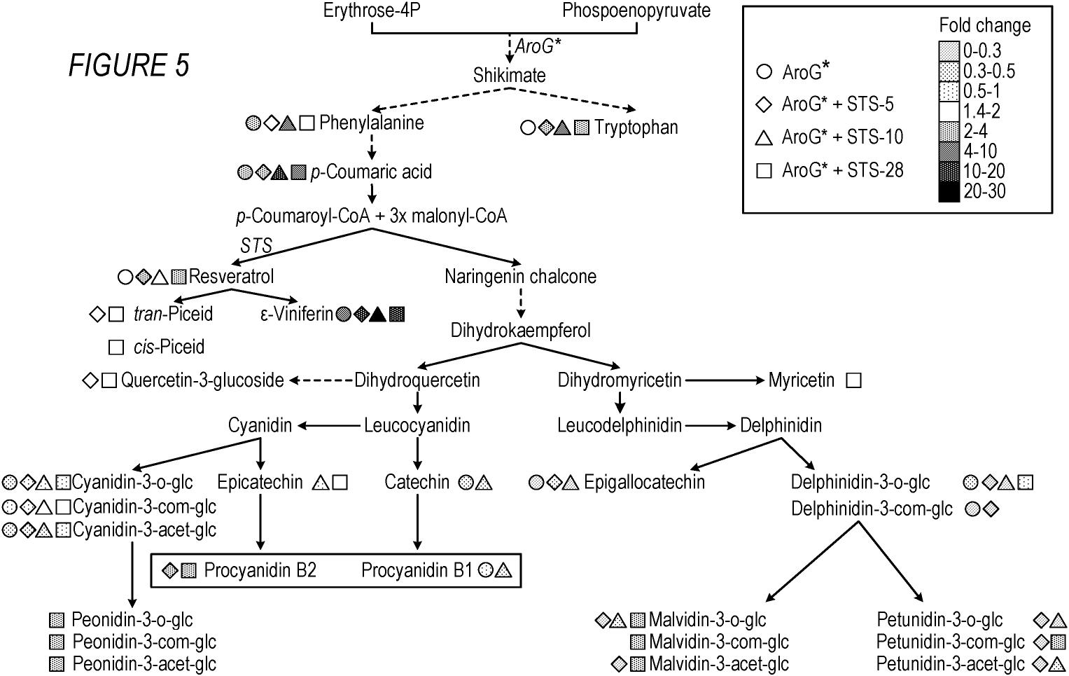

The transformed lines had a significant effect on the four annotated and quantified stilbenes, trans-piceid, cis-piceid, resveratrol and ε-viniferin. In the AroG* lines, resveratrol and ε-viniferin increased significantly in the transgenic lines in comparison to controls (FIG. 3, bottom panel, FIG. 5). Stilbene levels increased further in the double transformed lines (AroG* + STS) in comparison to AroG* alone: The levels of trans and cis-piceid increased in AroG* + STS5 and 28, and s-viniferin in AroG* + STS5 and 10 (FIG. 3, bottom panel, FIG. 5). Interestingly, resveratrol levels did not increase significantly due to the overexpression of any of the three STS genes in addition to AroG* (FIG. 3, bottom panel, FIG. 5).

Among the four identified stilbenes in the grape cell culture, the only one that increased significantly in all the transgenic lines tested is ε-viniferin (Table 2). The line with the highest levels of ε-viniferin was AroG* + STS10-182, with a 74-fold increase in comparison to controls, reaching a concentration of 0.74 mg/g dry weight (Table 2). In this same line, resveratrol levels were 9-fold higher than controls. The double transformed cell lines, increasing production of amino acids and directing the carbon flux to the stilbene biosynthetic pathway, accumulated higher concentrations of additional stilbenes, and in particular s-viniferin.

Stilbene content (mg/g dry weight) in each cell line of AroG* and AroG*+STSs, respectively. Values are presented as mean ± SE (n=8). In the control n = 24, including three lines. Numbers in bold font indicate a significant increase in transgenic lines compared to the control line (P < 0.05, Dunnett’s test).

TABLE 2

| | trans piceid | cis piceid | reservarol | viniferin |
|---|---|---|---|---|
| control | 0.20±0.01 | 0.20±0.01 | 0.05±0.05 | 0.01±0.01 |
| AroG^{•}-1 | 0.19±0.01 | 0.14±0.01 | 0.03±0.03 | 0.02±0.01 |
| AroG^{•}-3 | 0.42±0.05 | 0.39±0.04 | 0.64±0.06 | 0.50±0.047 |
| AroG^{•}-4 | 0.25±0.01 | 0.20±0.01 | 0.05±0.01 | 0.07±0.007 |
| AroG^{•}-16 | 0.18±0.02 | 0.13±0.01 | 0.26±0.06 | 0.03±0.006 |
| AroG^{•}+STTSS-2 | 0.48±0.07 | 0.43±0.05 | 0.30±0.06 | 0.66±0.093 |
| AroG^{•}+STDD-7 | 0.35±0.03 | 0.22±0.02 | 0.08±0.01 | 0.13±0.013 |
| AroG^{•}+STSS-71 | 0.29±0.03 | 0.22±0.02 | 0.07±0.01 | 0.05±0.007 |
| AroG^{•}+STS10-18 | 0.08±0.01 | 0.05±0.01 | 0.06±0.01 | 0.03±0.002 |
| AroG^{•}+STS10-59 | 0.26±0.02 | 0.18±0.01 | 0.08±0.01 | 0.24±0.018 |
| AroG^{•}+STS10-182 | 0.30±0.03 | 0.20±0.02 | 0.45±0.05 | 0.74±0.067 |
| AroG^{•}+STS28-6 | 0.35±0.02 | 0.27±0.02 | 0.14±0.02 | 0.05±0.006 |
| AroG^{•}+STS28-16 | 0.39±0.06 | 0.31±0.02 | 0.21±0.04 | 0.18±0.017 |
| AroG^{•}+STS28-71 | 0.45±0.05 | 0.27±0.03 | 0.12±0.02 | 0.20±0.024 |

In contrast, the levels of flavonoids and anthocyanins, having the same precursors as stilbenes, namely Phe and p-CA, were only mildly affected by the overexpression of AroG* or AroG* + STS, and in most cases their levels decreased in comparison to controls (FIG. 4, FIG. 5). The AroG* + STS28 lines are exceptional with increased levels in several anthocyanins and flavonoids, and AroG* + STS5 lines had higher levels of two flavonoid, quercetin-3-glucoside and procyanidin B2 (FIG. 4, FIG. 5).

### Example 3. Effect of Phe and p-CA Feeding of an AroG*+STS Transformed Line on Its Metabolic Profile

The metabolomics analysis of the transgenic AroG* and AroG* + STS lines clearly showed that substrate availability is a bottleneck in the production of stilbenes. To test whether further increase in precursors for production of stilbenes will results in even higher levels of stilbenes, transgenic grape cell lines were fed with either Phe or p-CA. The AroG* + STS28 transgenic lines were the most consistent in their effect on stilbene levels with all three lines having significantly higher viniferin, resveratrol and t-piceid stilbenes (Table 2). Therefore, the strongest AroG* + STS28 (line 16) was chosen to test the potential in further increasing stilbenes accumulation by feeding with the precursors of these metabolites.

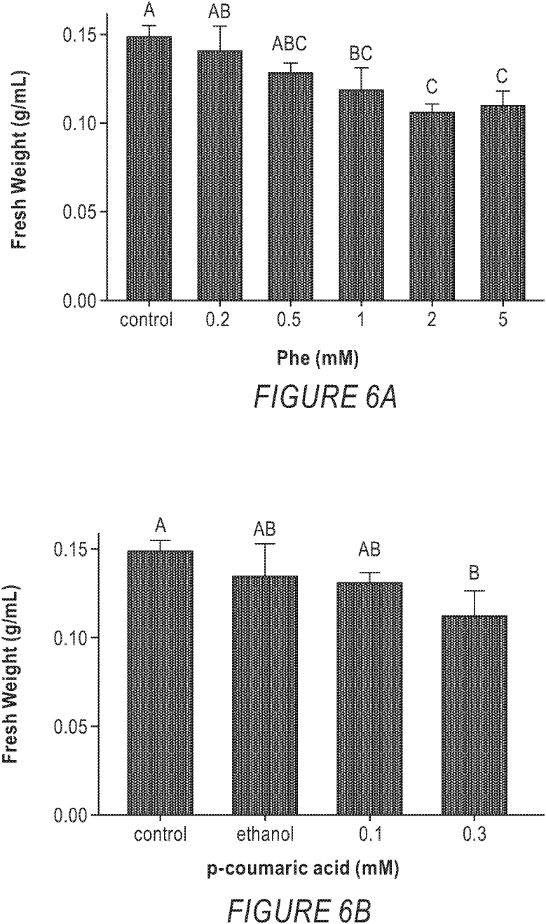

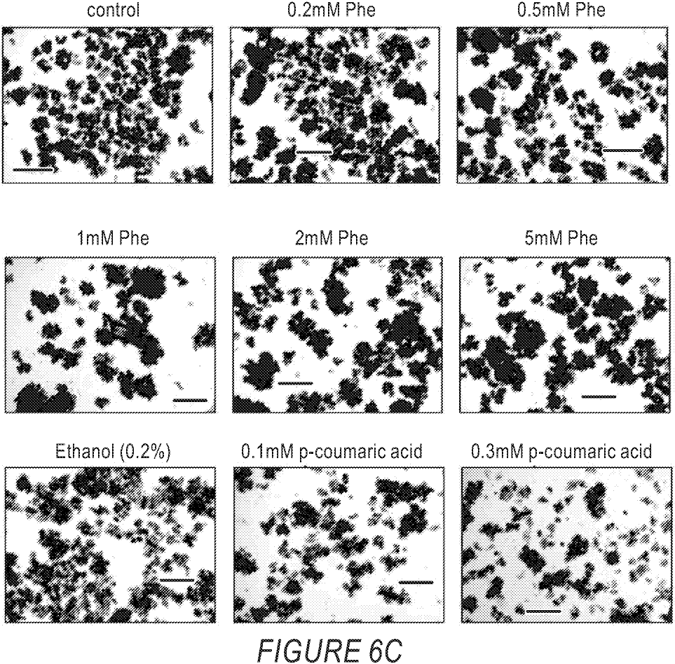

The concentrations of Phe and p-CA chosen for the feeding experiments were those in which cell viability was not affected, but cell growth was slower. To compare the growth rate, the fresh Weight (FW) of the cells per ml growth media was measured at day 7 of growth. The weight of Phe treated cells at the concentration of 0.2 and 0.5 mM at day 7 was similar to controls, whereas those grown in 1, 2 and 5 mM Phe weighed significantly less (FIG. 6A). Since p-CA was not water soluble, the effect of the treatments was compared to cells grown in 0.2% ethanol, with a slower growth of cells grown in 0.3 mM p-CA (FIG. 6B). Even though the viability of the treated with 0.5-5 mM Phe and 0.1-0.3 mM p-CA was similar, their growth pattern differed with formation of larger clumps in the liquid media (FIG. 6C).

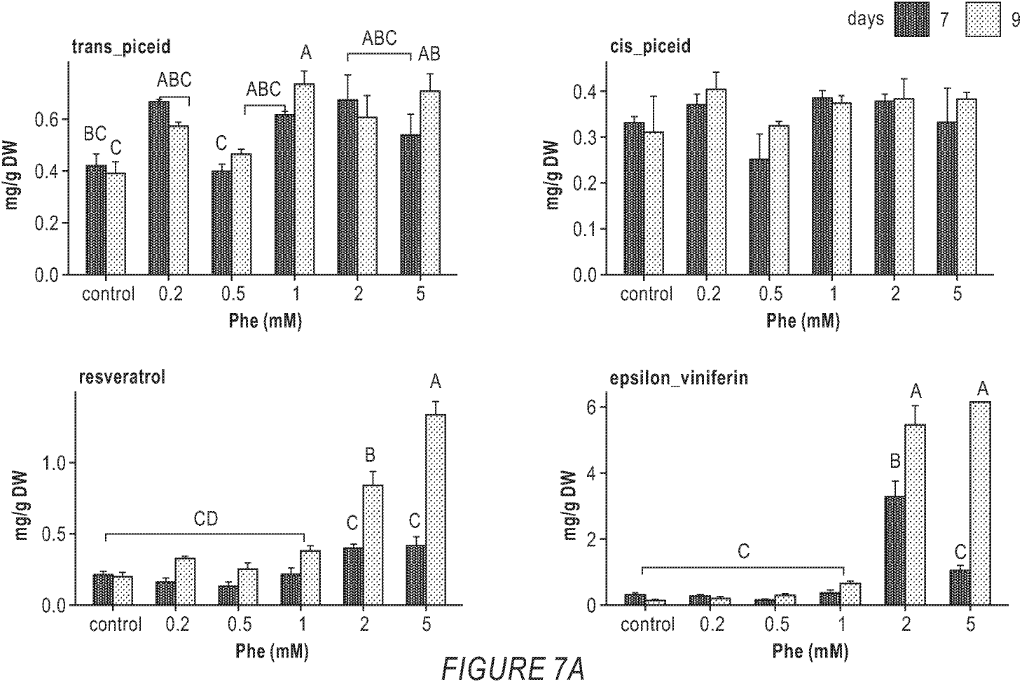

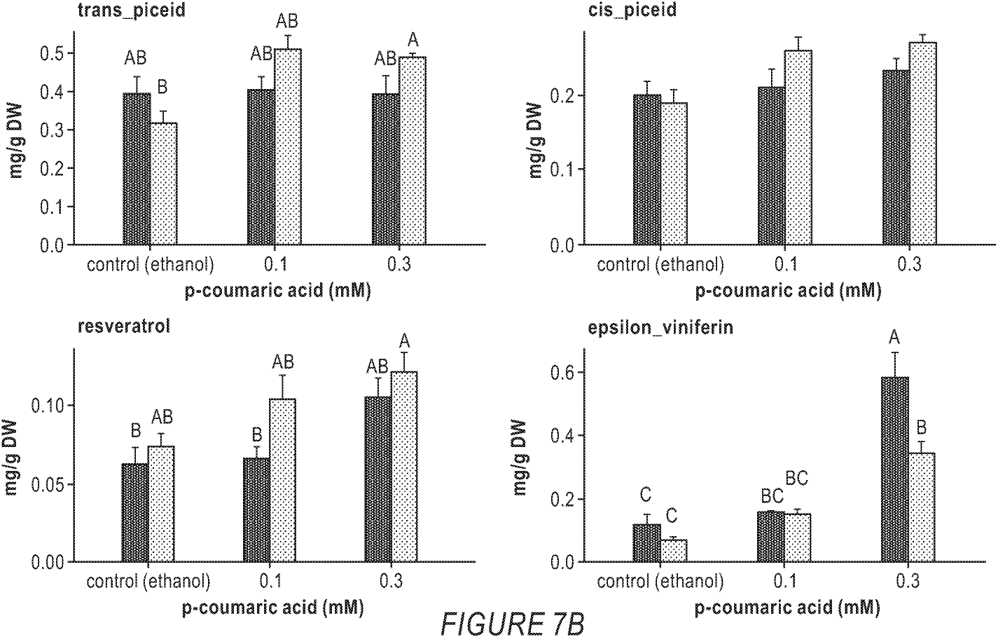

Phe feeding at 2 and 5 mM concentrations caused a significant increase in resveratrol and s-viniferin contents in the double transformed line AroG* + STS28-16 (FIG. 7A). The most significant increase for both stilbenes was at day 9, when the cell culture is known to accumulate the most polyphenols (Manela et al., 2015), when treated with 5 mM Phe. Resveratrol levels increased 6-fold resulting in a concentration of 1.26 mg/g dry weight, while s-viniferin levels increased 30-fold, up to 10.8 mg/g dry weight (FIG. 7A). Feeding of the AroG* + STS28-16 line with p-CA significantly increased the levels of stilbenes and in particular especially ε-viniferin, and the most significant increase for s-viniferin was at day 7 when treated with 0.3 mMp-CA (FIG. 7B).

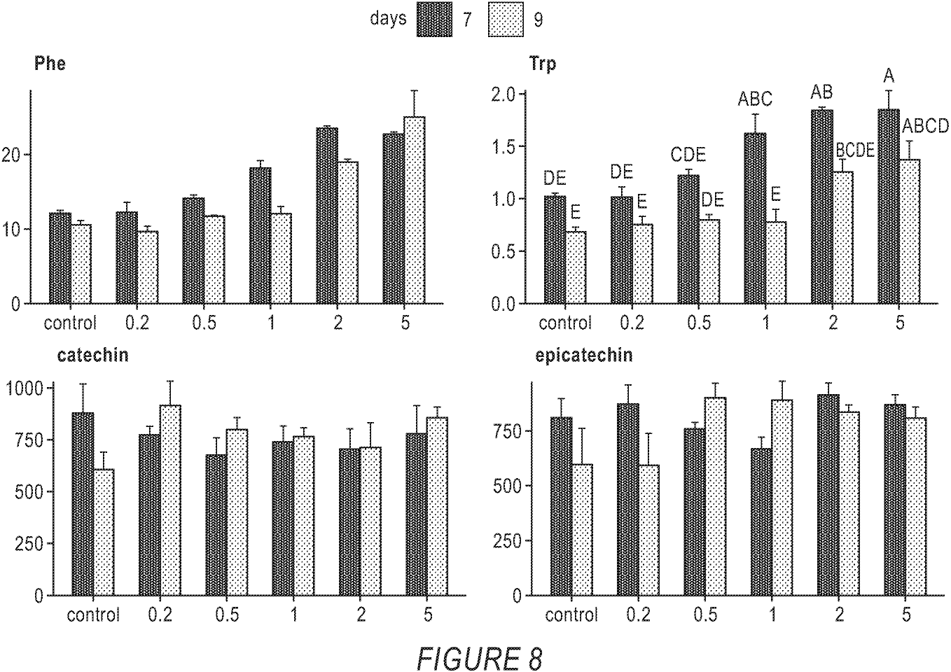

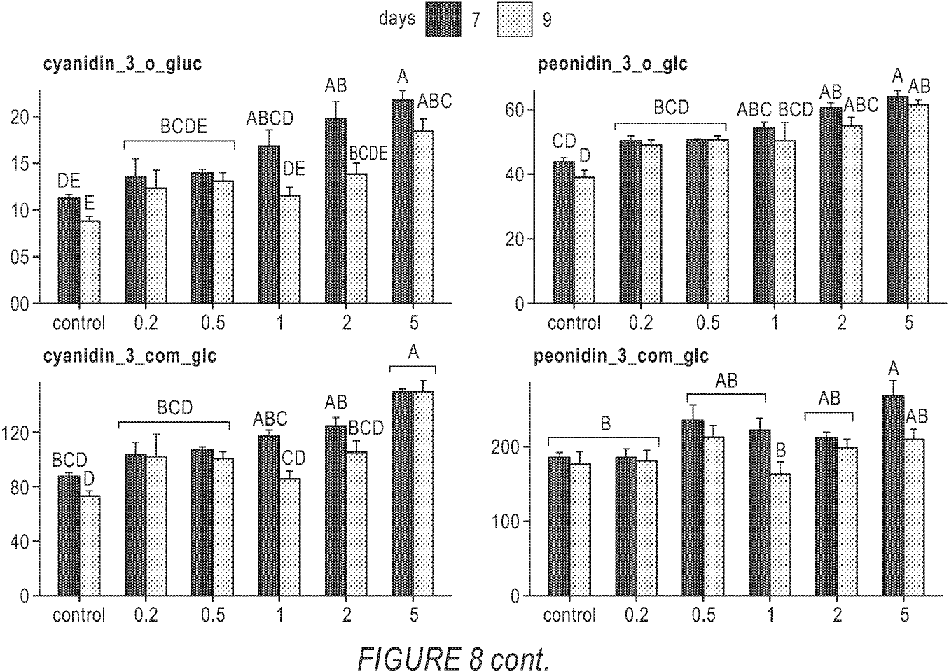

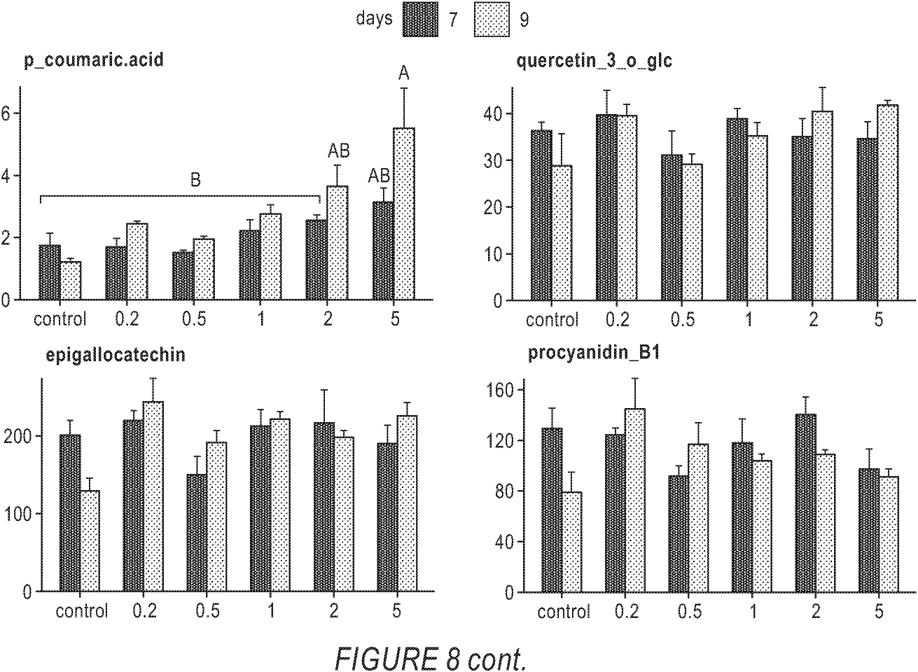

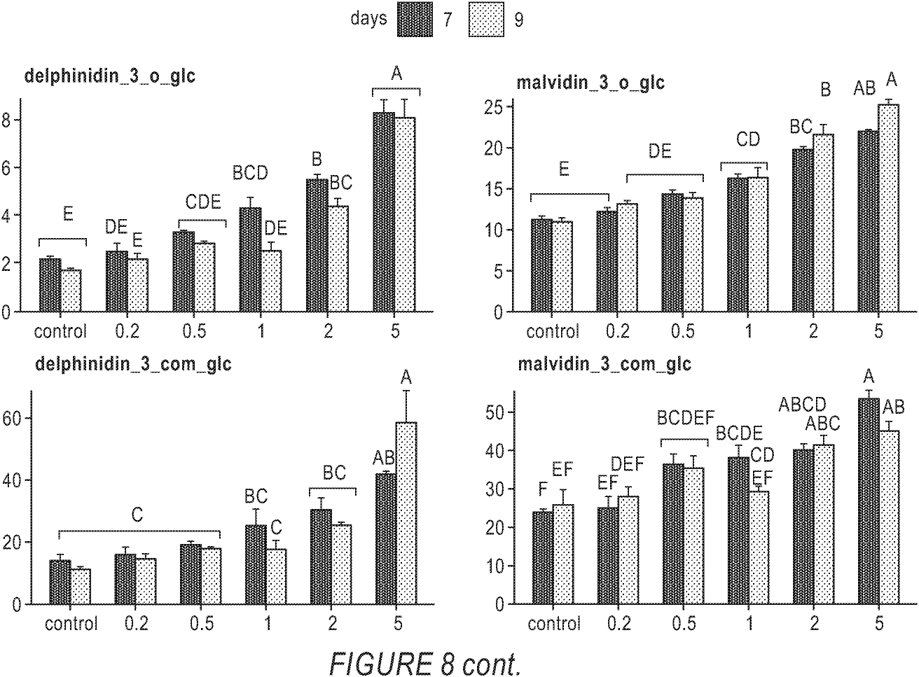

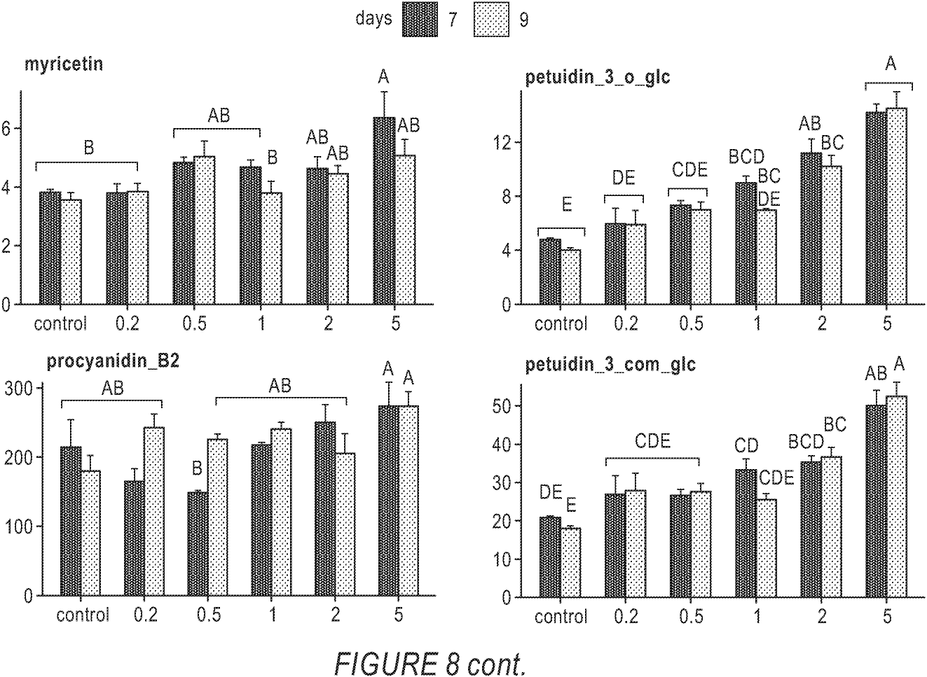

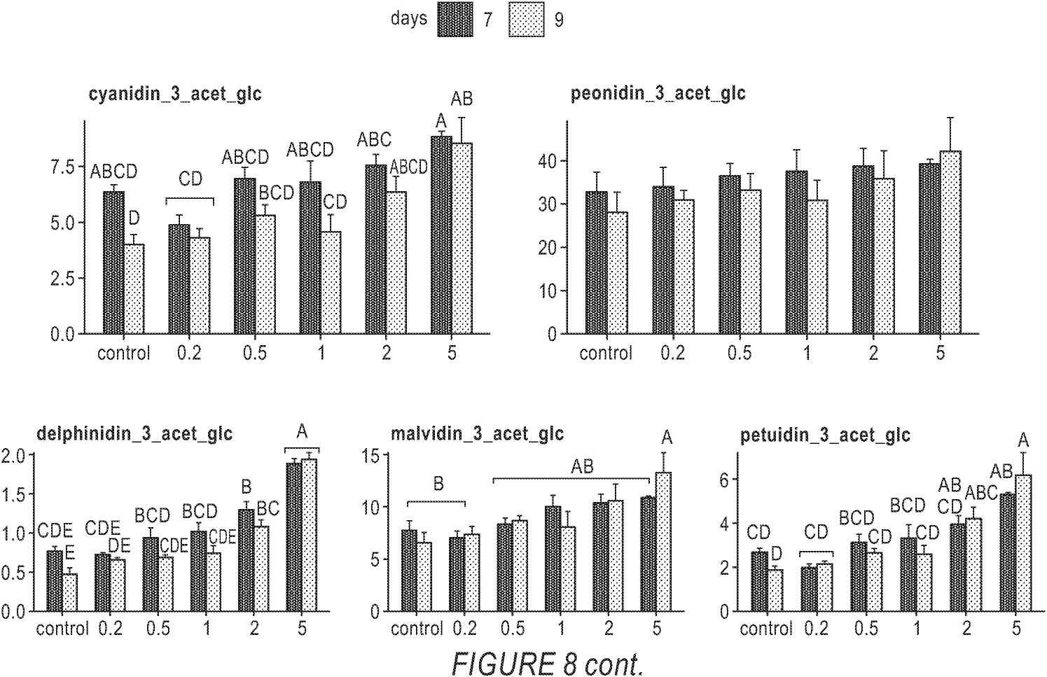

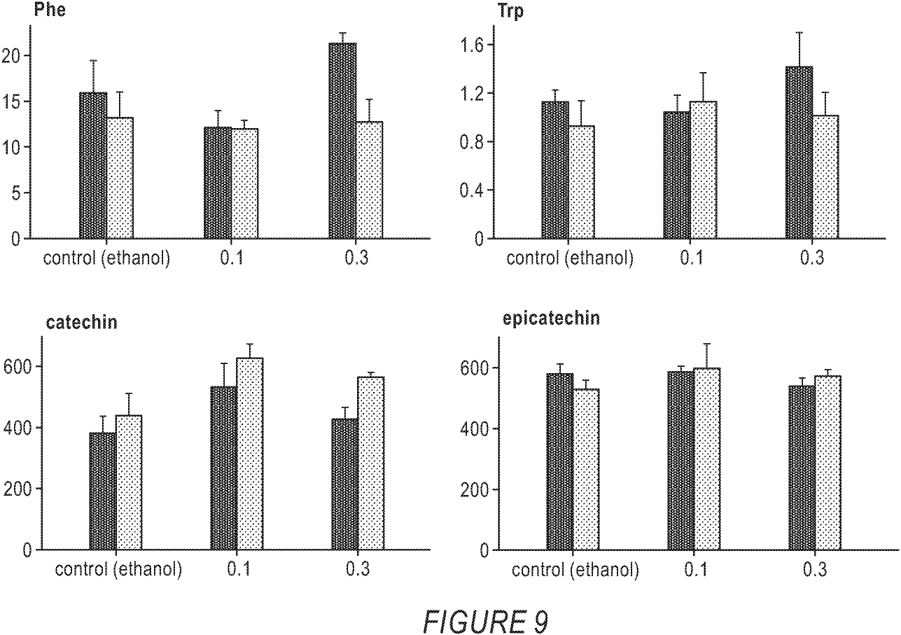

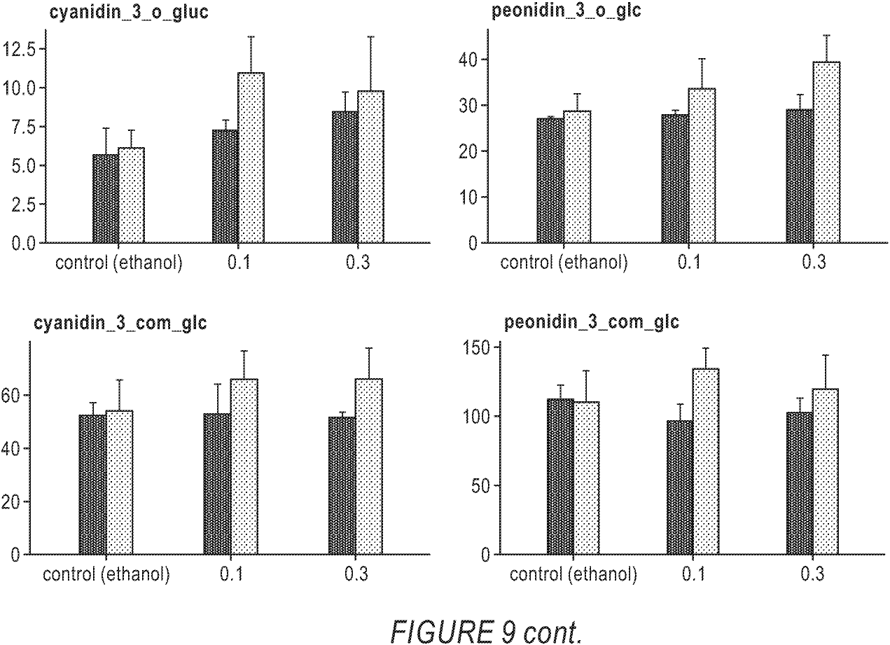

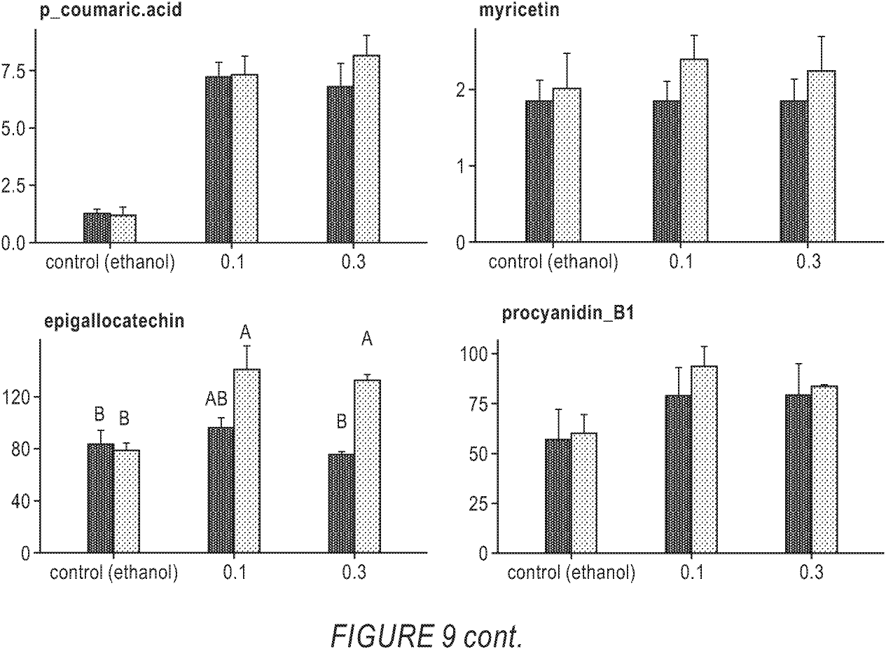

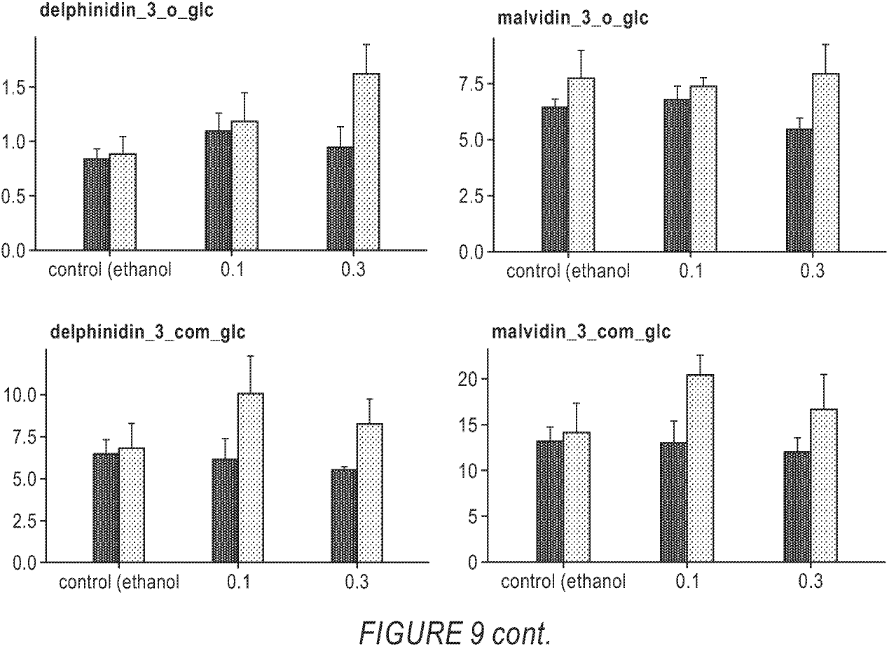

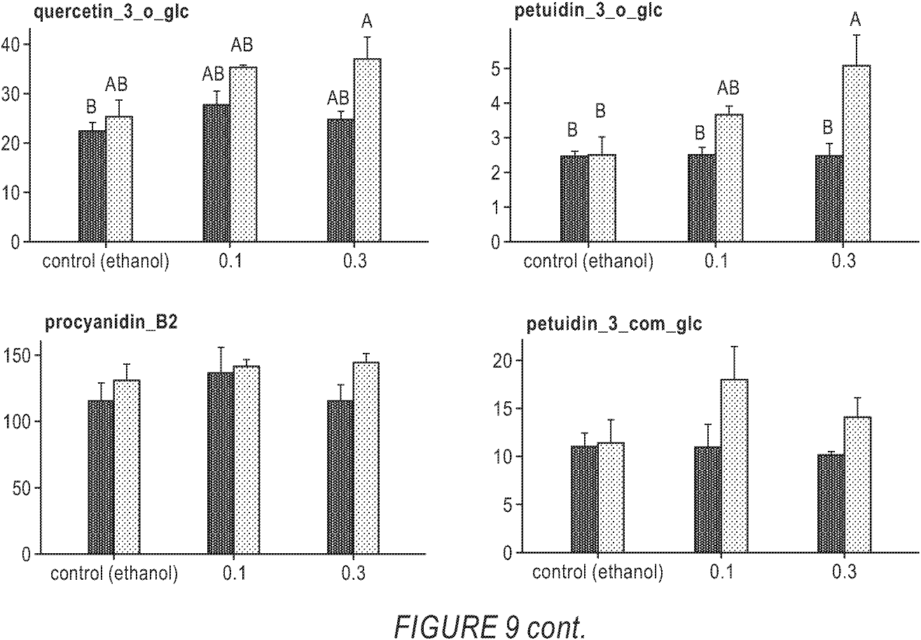

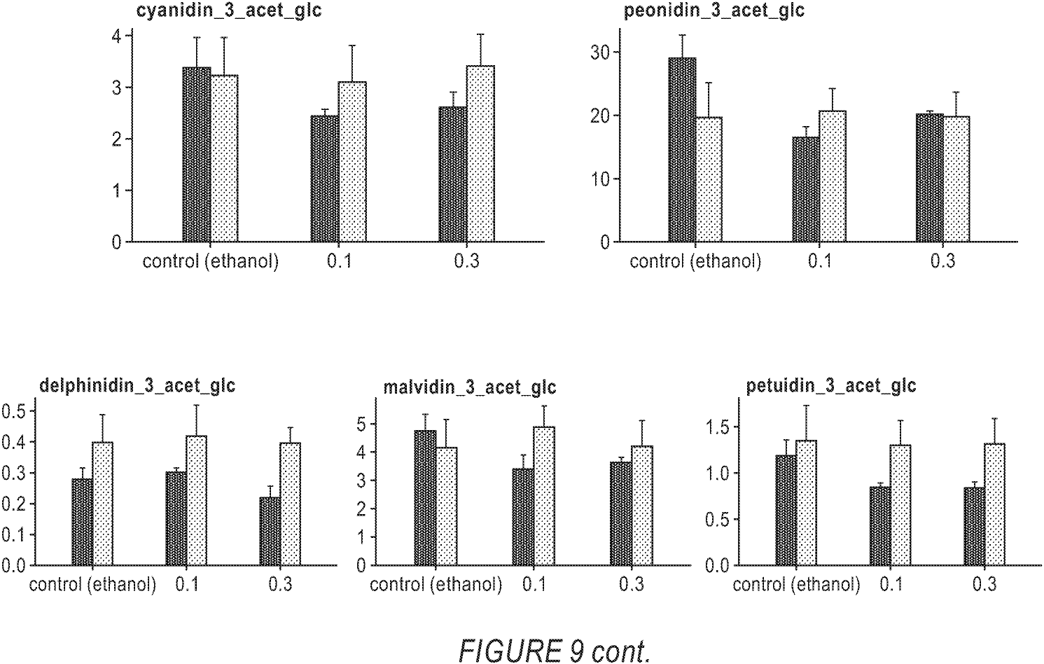

Phe and p-CA feeding of line AroG* + STS28-16 also caused a significant increase in in several flavonoids (FIG. 8 and FIG. 9). The levels of most anthocyanin and its derivatives were significant increase in 5 mM Phe fed cells compared to control (FIG. 8). Feeding of the AroG* + STS28-16 line with 0.3 mM p-CA had higher levels of two flavonoids, epigallocatechin and petunidin-3-O-glucose (FIG. 9).

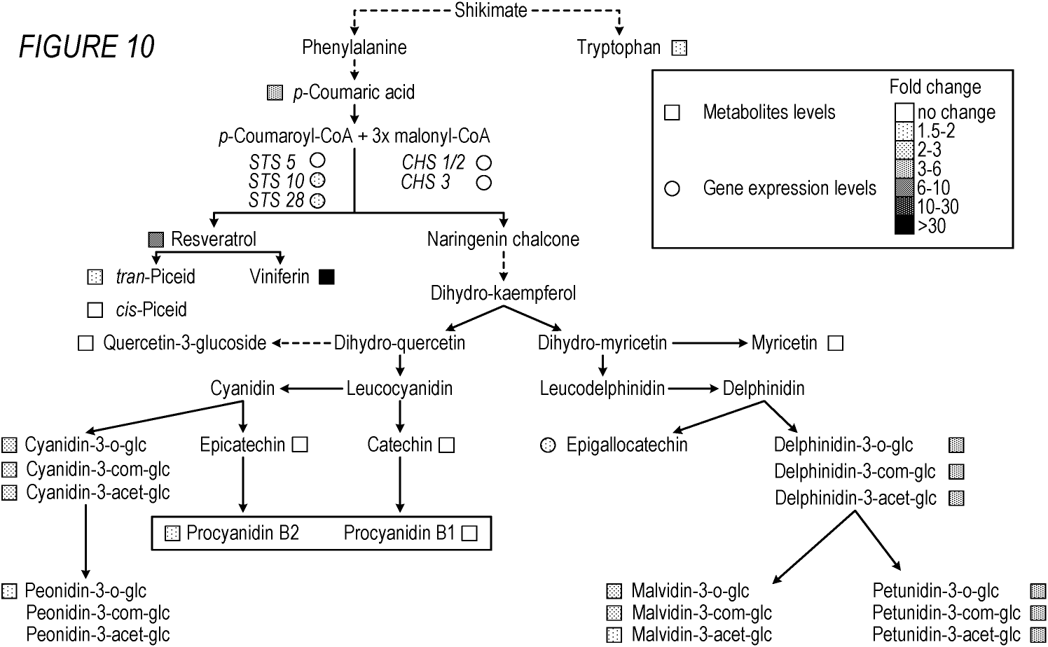

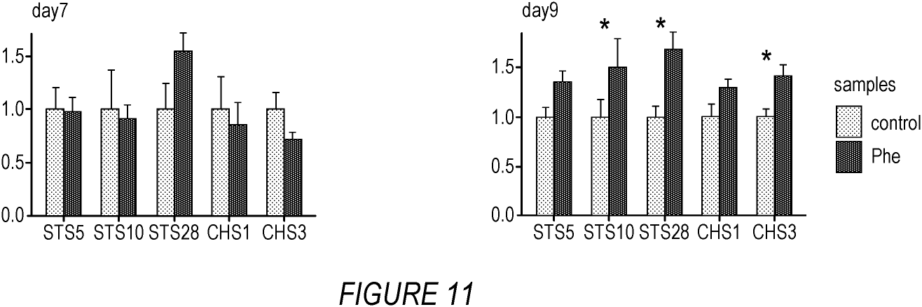

In summary, the strongest effect of on the production of stilbenes was that resveratrol and viniferin concentrations increased in line AroG* + STS28-16 around 24 and 600-fold respectively due to the transformation and feeding with Phe. This precursor feeding strategy based on transgenic line did not cause changes in gene expression of STS and Chalcone synthase or naringenin-chalcone synthase (CHS) (FIG. 10 and FIG. 11).

### Example 4. Attenuation of LPS-Induced Cytokine Storm in White Blood Cells (WBCs) of Chickens, by GCE

Rational: Since WBCs have a major role in Cytokine Storm burst, this part of the study provides basic data for better focused experiments in-vivo (Example 5).

Experimental design: Grape cells were collected at day 9 and washed twice with cold dH2O, lyophilized and frozen in -80° C. (GCP). Polyphenols were extracted from the GCP as follows: samples were homogenized in a frozen mixer mill with metal beeds. Then, pre-cooled 70% methanol was added to the tube. Samples were incubated for 20 minutes at room temperature on an orbital shaker and centrifuged at full speed for 10 minutes, and the supernatant was transferred to a new tube (GCE in 70% methanol). The supernatants were vacuum-dried in a Speed Vac Concentrator at room temperature and dissolved in 50% DMSO. The final solution was stored in -80° C. for further analysis and use (GCE in 50% DMSO).

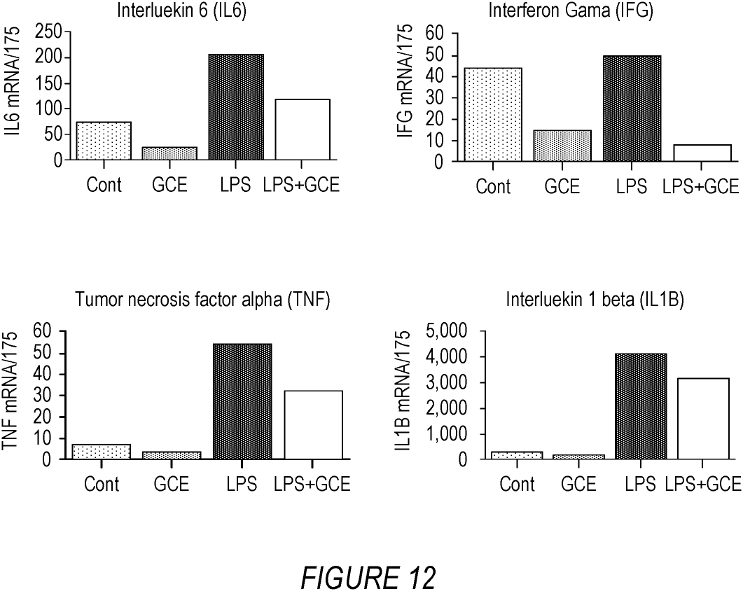

Male layer-type chickens are purchased and grown. On day 14 of age, blood is drawn and WBC are purified, and treatment with GCE and LPS as described in FIG. 12 but with a larger range of GCE doses (corresponding to 0.1 to 1 mg/ml of powder) loner and shorter pre-intubation periods (0 to 16 hours) and a more complete variety of cytokine probes. Several experiments are used to optimize the Cytokine Storm amelioration by the GCE, in 3 duplicates. The optimized protocol is further used to compare the efficiency of the new preparation of GCE with the older preparation employed in studies in-vivo (in rats) thus helping estimate the range of effective dose for the in-vivo studies.

Results: Based on preliminary results, these experiments enable detailed characterization of the GCE effect on WBCs, establishing the ground for the in-vivo studies.

### Example 5. Attenuation of LPS-Induced Cytokine Storm in Chickens, by Feeding With GCP

Rational: LPS challenge is a well-characterized model for Cytokine Storm in both mammals and chickens, thus providing a good assay system for Cytokine Storm inhibition by GCE in-vivo. This set of experiments enables a more focused study with less unknown variants in the experiments of Example 6.

Experimental design: 20 Broiler type chickens at weeks of age (purchased from Brown and sons, LTD) are randomly divided into 2 groups of 10 birds. Chicks of the treatment group were fed with grape cell powder (GCP; ~ 170 mg/Kg BW/day) for 7 days. The control group was fed with the regular formula. On the 7th day, 2 hours before killing, lipopolysaccharides (LPS; 1 mg/Kg BW) were injected to the wing vein of all chicks. Spleens were excised immediately after killing by neck dislocation and kept in RNA-Later. RNA was extracted using total RNA minikit (Geneaid) and mRNA expression level was analyzed by qPCR according to standard procedure.

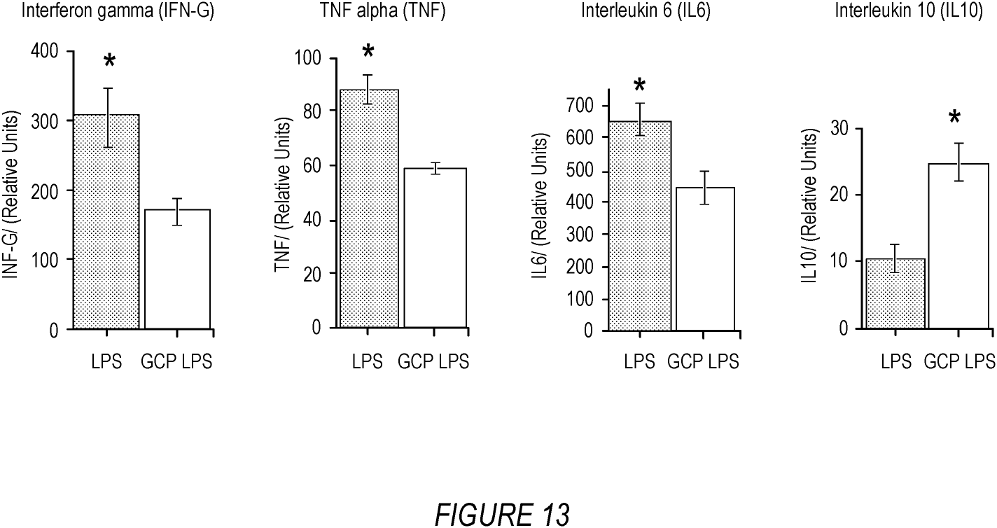

Results: Feeding chickens with GCP ameliorates the induction of Cytokine Storm surge. FIG. 13 shows inhibition of mRNA expression level of pro-inflammatory cytokines (INF-G, TNF, IL-6) and induction of the anti-inflammatory cytokine mRNA (IL-10), in spleens of chickens fed with grape-cell-powder for 7 days and stimulated by LPS for 2 hours, as compared with spleens from LPS fed chicks.

In summary, feeding with grape-cell-powder (GCP) decreased pro-inflammatory cytokines and induced an anti-inflammatory cytokine. These results support the use of GCP for the prevention of CRS or cytokine storm.

### Example 6. Attenuation of IBV-Induced Cytokine Storm and Disease Symptoms, By Feeding With GCP

Experimental design: Male chicks are purchased and maintained. At day 14 of age the birds are infected by attenuated IBV vaccine (H-120, Biovac, Or-Akiva, Israel) at 4-fold and 8-fold higher doses, compared to regular vaccination, directly into the chick’s air sacs at both sides. The clinical signs of the IBV infection (coughing, rattling, body temperature) are recorded daily until recovery or death, and viral load is determined by RT-qPCR, in tracheal swab samples at day 5 and 10 following virulent IBV challenges. After choosing the optimal infection conditions, experiments employing GCP treatment of IBV infection are performed at a similar experimental-design described in Example 5 but using only the optimized GCP dose and treatment schedule. The number of birds/group is tripled to include a group for longer term following of disease symptoms, in addition to the characterization of Cytokine Storm amelioration. Tissue sampling is as in Example 5 with the addition of lung and trachea. All tissues samples are divided to 3 for RNA analysis by qPCR, as well as protein mass spectrometry and RNA-seq that are performed only for the experiment with the best Cytokine Storm inhibition.

Results: The high-dose-vaccination induces disease symptoms with high similarity to the human SARS-CoV family of human coronaviruses, and feeding with the GCP reduces both Cytokine Storm and the severity of the disease symptoms.

### Example 7. Attenuation of IBV-Induced Cytokine Storm (CS) and Disease Symptoms, By GCE Applied Via Alzet Osmotic Pumps

Rational: One of the strengths of this invention is the grape cell-line overexpressing stilbenes, primarily epsilon viniferin, known with its strong anti-inflammatory activity. To ensure maximal bioavailability of the unique stilbenes mixture and their synergistic effect, the effect of application by feeding to a slow release system using Alzet osmotic pump is tested.

Experimental design: Osmotic pump (ALZET®; 2ML4 pumps, allowing flow of 2.5 µl/hour), filed with GCE is implanted subcutaneously at the rejoin of the 7th cervical vertebrae. Timing of implantation and IBV infection is determined based on the optimization experiments in Example 5 and Example 6.

Results: A higher CS-ameliorating effect by GCE.

### Example 8. Generation of Transgenic V. Vinifera Cv. Gamay Red Cell Culture Co-Expressing AroG* and FLS Genes

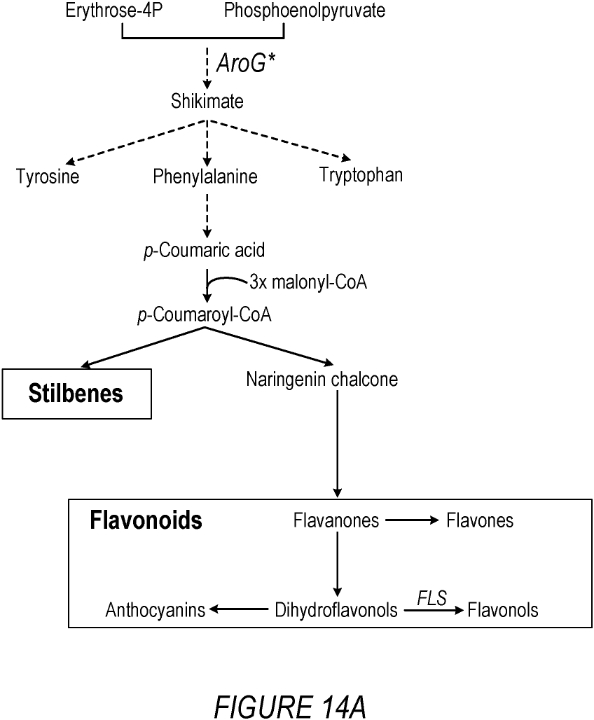

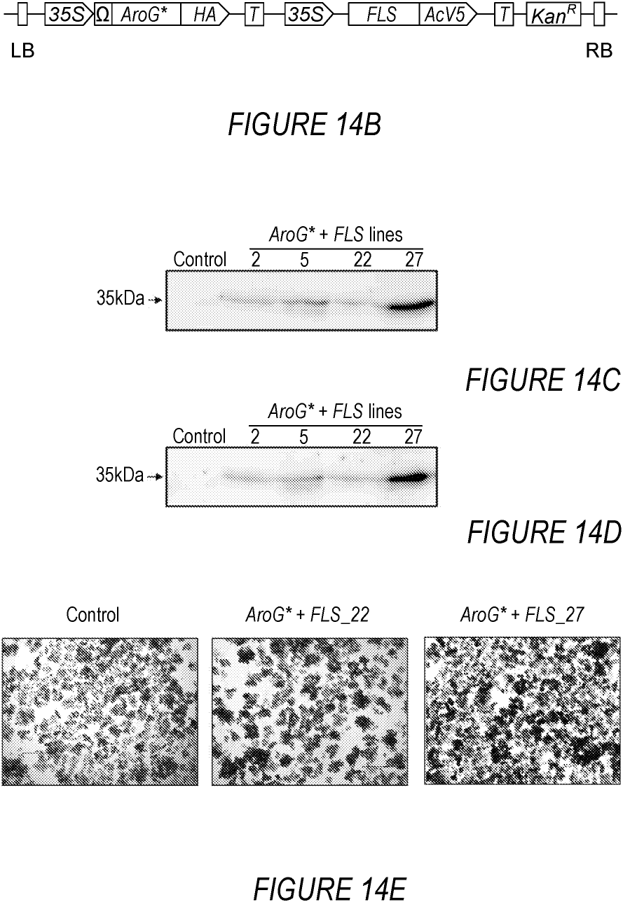

In an attempt to divert the carbon flux towards flavonoid production, the V. vinifera cv. Gamay Red grape cells were transformed with a construct including both AroG* and Flavonol synthase (FLS), under 35S promotors (FIG. 14A, B). Among the six FLS genes identified in grape (Anesi et al., 2015; Fugita et al., 2006), the one chosen for transformation was VIT_07s0031g00100 (NCBI: XM_002283156) since it is the only FLS gene expressed in the V. vinifera Gamay Red cell culture. The FLS gene was cloned for preparing the construct including both AroG* and FLS (FIG. 14B), for transformation of the grape cell culture.

Independently transformed cell lines were analyzed for the accumulation of AroG* and FLS proteins (FIG. 14C, D). Four of the ten transgenic lines expressing both proteins were chosen, based on their vigor, for a wide metabolomic analysis (FIGS. 14C-E).

### Example 9. Effect of Co-Expression of AroG* and FLS Genes on The Metabolic Profile Of Grape Cell Culture

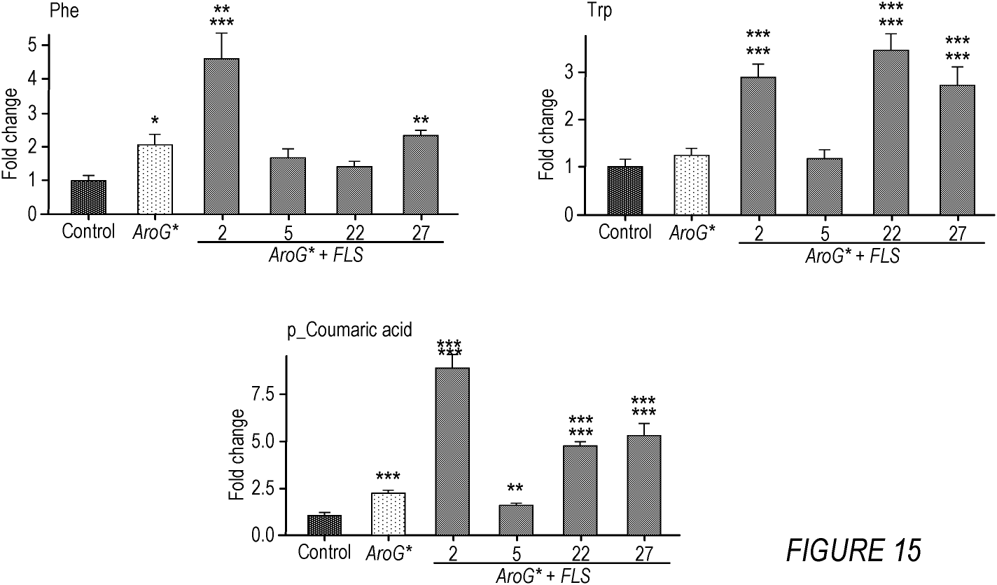

Similar to the AroG* line, the transformed AroG* + FLSlines accumulated higher Phe and p-coumaric acid (p-CA) levels in comparison to the control (FIG. 15). Both metabolites are common precursors for stilbenes and flavonoids. Furthermore, several of the AroG* + FLS lines had even higher levels of Phe and p-CA than the AroG* line and in addition accumulated high levels of Tryptophan (Trp) (FIG. 15).

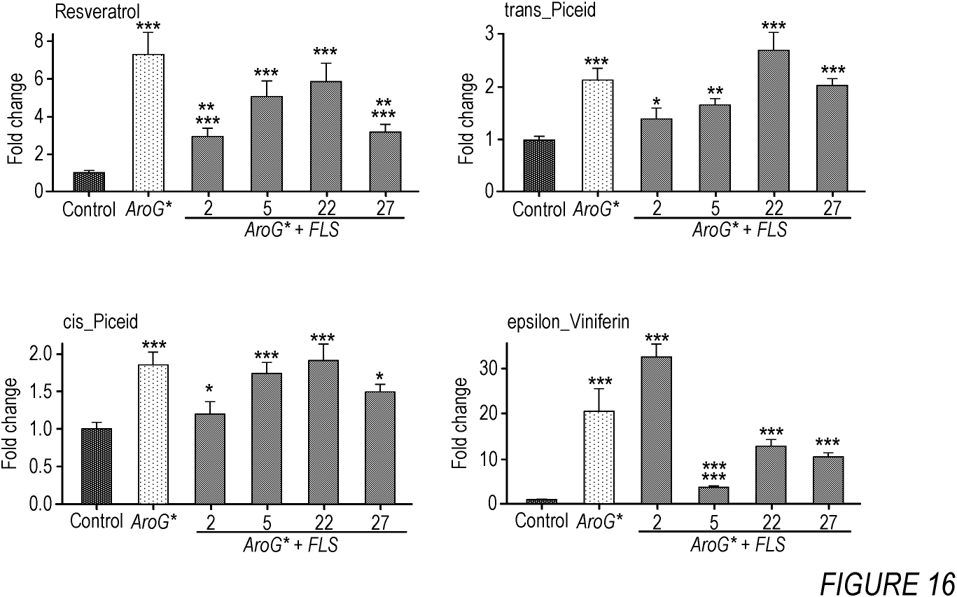

Interestingly, the co-expression of AroG* + FLS resulted in increased levels of stilbenes in the grape cell culture, similar to that caused by transformation with AroG* alone (FIG. 16). This was true, even though the FLS gene is part of the flavonoid biosynthetic pathway. In most cases, the levels of the four different stilbenes identified in the cell cultures, resveratrol, trans and cis piceid, and ε-viniferin, were similar to that of the single AroG* transformed line, and higher than that of the control. Resveratrol levels increased approximately a 6-fold (0.3 mg/g DW), and viniferin levels about 30-fold (0.3 mg/g DW), while the other stilbenes increased to lower levels (FIG. 16). Clearly, co-expression of AroG* + FLS did not reduce the metabolic flux towards stilbenes.

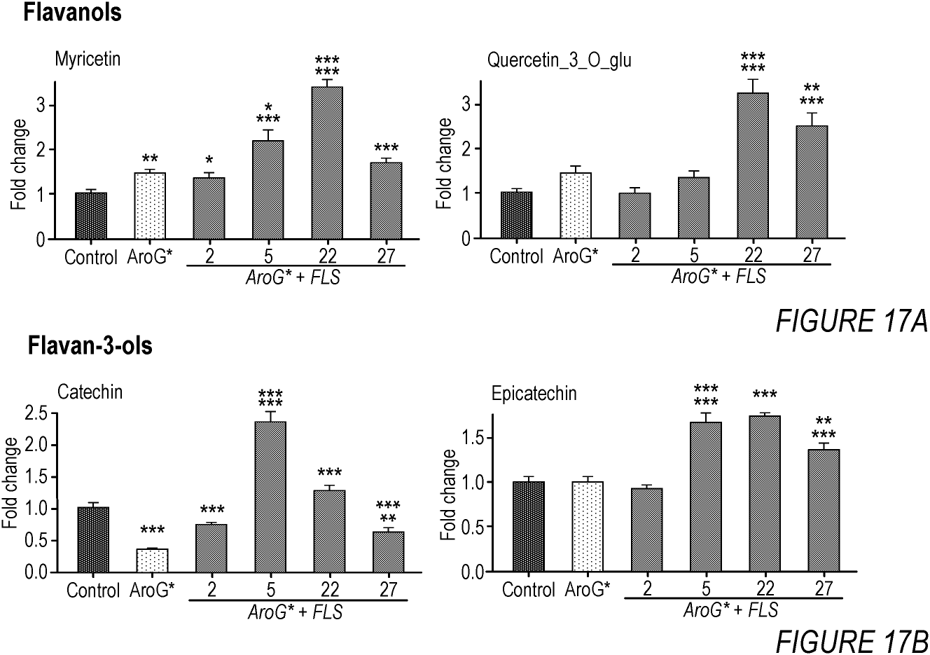

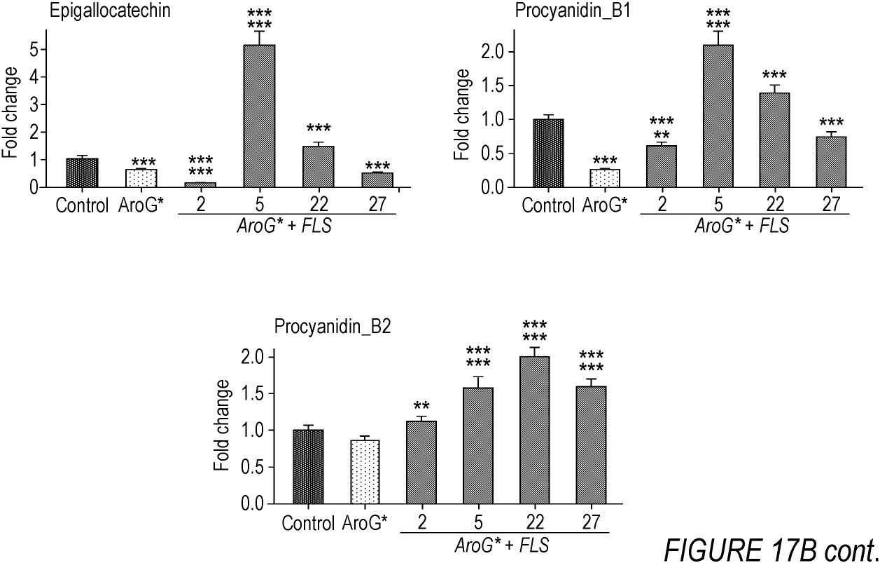

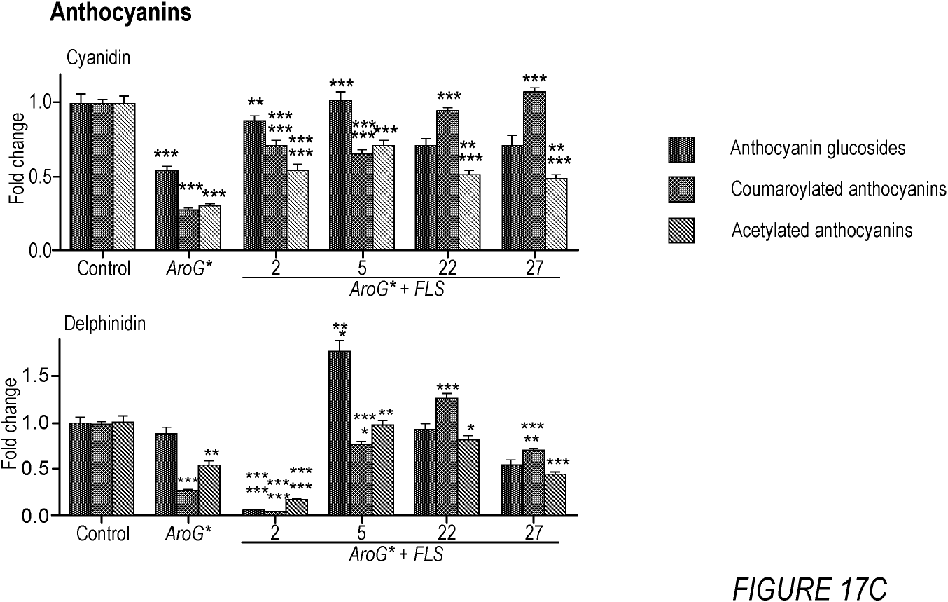

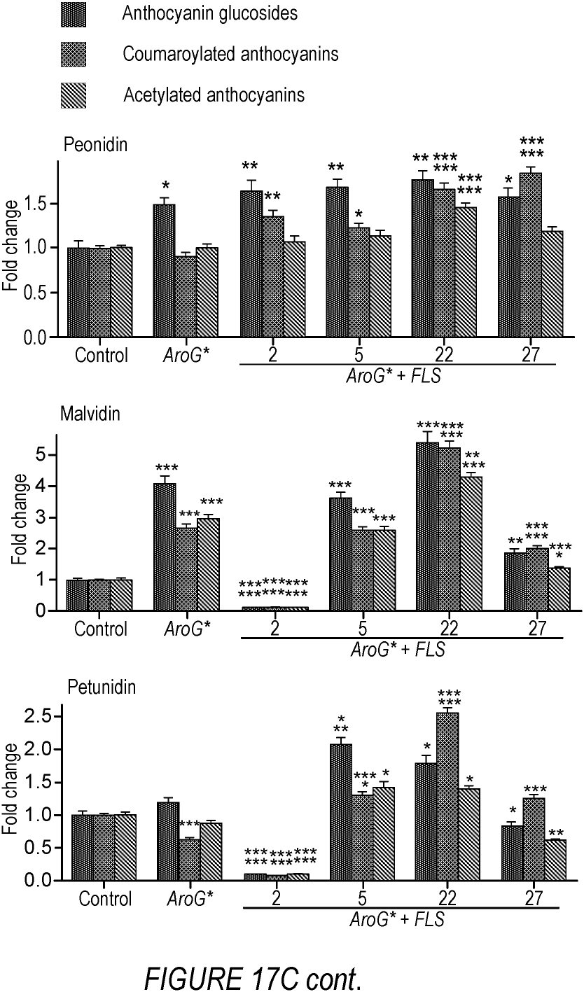

AroG* + FLS transformation also resulted in increased flavonoid levels in the grape cell culture. The levels of the two direct products of the FLS enzyme, the flavonols myricetin and quercetin-3-O-glucose, increased significantly (FIG. 17, panel (a)). Two of the four AroG* + FLS lines accumulated significantly higher levels of flavonols (up to 3.5-fold) in comparison to the control and single AroG* lines. Additional flavonoids increased due to the co-expression, including flavan-3-ols and anthocyanins, to higher levels than both the AroG* and control lines (FIG. 17).

Flavonoids levels were either lower or the same as controls in the AroG* line, except for malvidin, which was higher in the AroG* line. The AroG* + FLS lines had significantly higher levels of flavonoids, including 5 identified flavan-3-ols and 15 anthocyanin glucosides, in comparison to AroG* and in some cases in comparison to controls (FIG. 17 panels (b), (c)). Of the four AroG*+FLS lines, line 2 was exceptional in having very low levels in several of the flavan-3-ols and anthocyanins. Interestingly, this line had exceptionally high levels of Phe, p-CA and viniferin (FIGS. 15, 16).

These metabolomic results demonstrate that transformation of the V. vinifera cv. Gamay Red grape cell culture with AroG* + FLS increased the total carbon flux towards phenylpropanoid production in comparison to the AroG * line. Specifically, stilbenes accumulated to similar levels in both cases, in addition to increased flavonoids levels in the AroG* + FLS lines.

### Example 10. Effect of Phe Feeding on Stilbene and Flavonoid Levels in an AroG*+FLS Transformed Line

Phe availability is a rate-limiting factor in the production of stilbenes in the Gamay Red cell culture. The AroG* + FLS lines accumulated high levels of both stilbenes and flavonoids. To test for a further increase in the production of both phenylpropanoid sub-groups the cell culture was fed with Phe.

The AroG* + FLS line chosen for Phe feeding experiments was that with the highest levels of both stilbenes and flavonoids (line 22). Phe feeding (5 mM) in control, AroG* and AroG* + FLS- 22 lines increased both stilbenes and flavonoids in all three lines, and in particular, in the AroG* + FLS-22 line (Table 3).

TABLE 3

Effect of Phe feeding on the phenylpropanoids, flavonoids, stilbenes levels of control, AroG* and AroG* + FLS.

| | | Control | AroG* | AroG* + FLS |
|---|---|---|---|---|
| | Phe | 2.35±0.36 | 3.86±0.58 | 3.72±0.06 |
| | Trp | 0.81±0.12 | 3.02±0.05 | 6.68±0.87 |
| | p-Coumaric acid | 5.23±0.81 | 1.64±0.21 | 18.31±3.54 |
| Flavonols | Myricetin | 1.2±0.17 | 1.79±0.27 | 4.38±0.15 |
| | Quercetin-3-glu | 0.86±0.05 | 1.16±0.1 | 2.87±0.26 |
| Flavan-3-ols | Catechin | 1.56±0.22 | 0.89±0.16 | 1.52±0.24 |
| | Epicatechin | 0.67±0.06 | 0.89±0.12 | 1.28±0.24 |
| | Epigallocatechin | 1.17±0.16 | 1.15±0.14 | 1.76±0.34 |
| | Procyanidin B 1 | 1.28±0.14 | 0.97±0.04 | 1.4±0.31 |
| | Procyanidin B2 | 0.9±0.07 | 1.37±0.24 | 2.05±0.44 |
| Anthocyanins | Cyanidin-3-glu | 1.35±0.17 | 0.92±0.08 | 1.69±0.24 |
| | Cyanidin-3-acet-glu | 1.01±0.14 | 0.71±0.09 | 1.42±0.08 |
| | Cyanidin-3-coum-glu | 1.3±0.15 | 0.62±0.1 | 2.32±0.38 |
| | Peonidin-3-glu | 1.3±0.11 | 1.68±0.12 | 2.31 ±0.17 |
| | Peonidin-3-acet-glu | 0.74±0.06 | 1.25±0.14 | 2.16±0.26 |
| | Peonidin-3-coum-glu | 1±0.15 | 1.11±0.15 | 2.69±0.42 |
| | Delphindin-3-glu | 2.72±0.38 | 3.07±0.12 | 5.7±0.73 |
| | Delphindin-3-acet-glu | 3.95±0.92 | 2.8±0.28 | 3.43±0.12 |
| | Delphindin-3-coum-glu | 2.41±0.36 | 1.52±0.29 | 6.55±0.87 |
| | Malvidin-3-glu | 1.83±0.36 | 6.82±0.48 | 9.63±0.54 |
| | Malvidin-3-acet-glu | 0.89±0.12 | 4.75±0.58 | 7.06±0.85 |
| | Malvidin-3-coum-glu | 1.07±0.16 | 6.6±0.76 | 11.55±1.84 |
| | Petunidin-3-glu | 2.09±0.29 | 2.2±0.22 | 5.48±0.63 |
| | Petunidin-3-acet-glu | 1.99±0.37 | 2.59±0.44 | 5.23±0.57 |
| | Petunidin-3 -coum-glu | 1.59±0.02 | 2.04±0.32 | 9.64±1.27 |
| Stilbenes | t-Piceid | 1.39±0.12 | 3.06±0.1 | 3.66±0.13 |
| | cis-Piceid | 0.76±0.05 | 1.17±0.08 | 1.66±0.12 |
| | Resveratrol | 4.76±0.36 | 23.46±2.26 | 17.45±0.24 |
| | ε-Viniferin | 4.35±0.13 | 30.79±4.65 | 24.79±3.22 |

Values (mean ± SE, n = 3) are the fold change as compared to non-fed control. Shaded metabolites indicate a significant increase in these values using one-way ANOVA followed Dunnett’s test (P < 0.05). Metabolites in bold are those with higher levels in the AroG* + FLS line in comparison to the AroG* line, analyzed by student’s t-test (P < 0.05). Abbreviations: acet, acetyl; glu, glucoside; coum, coumaroyl.

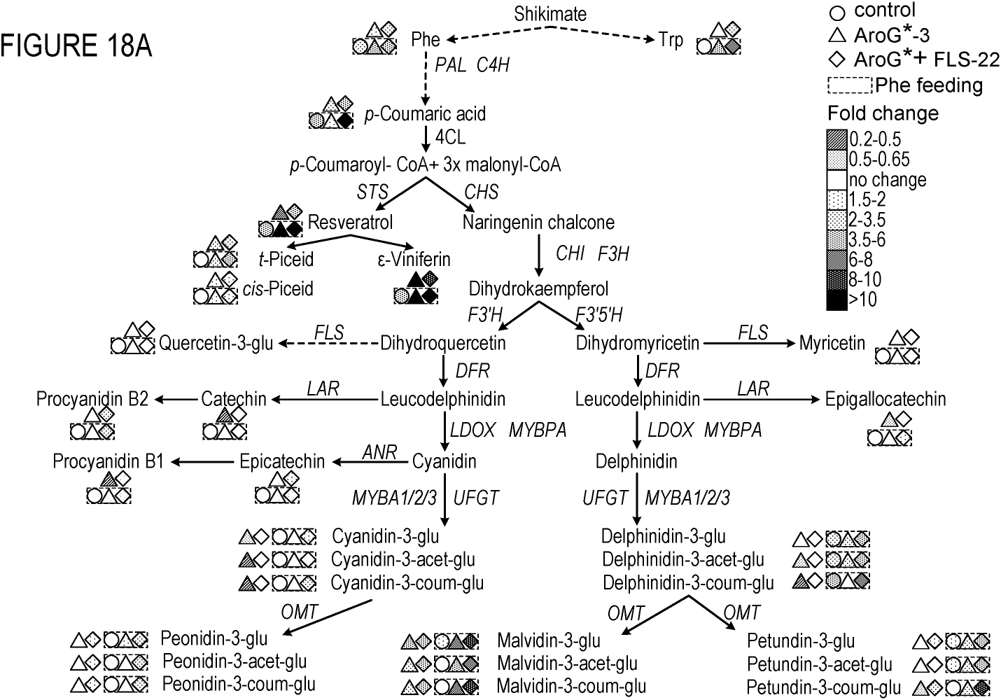

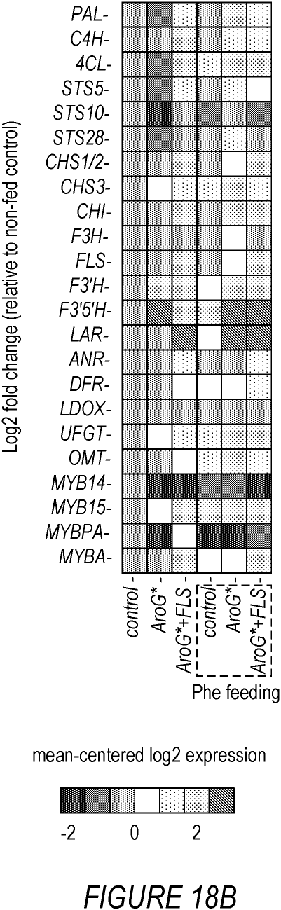

Phe levels in the cells increased due to exogenous feeding in all three lines. Both Trp and p-CA increased in the AroG* + FLS- 22 line to significantly higher levels than in both the control and the AroG* line (Table 3). Stilbenes, and in particular resveratrol and viniferin, increased significantly in all lines due to Phe feeding, with a similar and dramatic increase in the AroG* and AroG* + FLS-22 lines (Table 3, FIG. 18A).

Phe feeding caused a significant increase in the two products of the FLS enzyme, flavonols myricetin and quercetin-3-glucoside, in the AroG* + FLS- 22, as well as a smaller increase in the flavan-3-ols in line AroG* + FLS- 22 (Table 3). In addition, anthocyanin levels also increased in all three lines, mainly in the delphinidin-based anthocyanins (Table 3, FIG. 18A). The fold change increase in anthocyanins was significantly higher in line AroG* + FLS- 22.

The AroG* line had significantly higher levels of stilbenes, that was further enhanced due to Phe feeding. The only anthocyanins with higher levels in the AroG* line, malvidin-based anthocyanins, increased slightly more due to Phe feeding (FIG. 18A). In the AroG* + FLS- 22 line, stilbenes levels increased in comparison to the control and similar to the AroG* line, and anthocyanins levels increased significantly in comparison to both lines. Phe feeding of the AroG* + FLS- 22 line enhanced this effect and resulted in a further increase of both stilbenes and flavonoids (FIG. 18A).

### Example 11. Examine Whether Metabolomic Changes Due to AroG* + FLS Transformation and Phe Feeding are Dependent on Changes in Gene Expression Levels

The effect of transformation with AroG*, AroG* + FLS- 22 and Phe feeding on gene expression levels in the grape cell culture, was tested via quantitative real-time PCR analysis. The expression levels of genes along the biosynthetic pathways of stilbenes and flavonoids as well as transcription factors are known to affect these pathways were determined. Among the 48 VvSTS genes in grape, three were selected for gene expression analysis (VvSTS5, VvSTS10 and VvSTS28) based on the fact that when these three genes were transiently expressed in Nicotiana benthamiana leaves, they caused the highest increase in several stilbenes (Parage et al., 2012). Furthermore, these three STS genes represent the three main VvSTS sub- phylogenetic families (Vannozzi et al., 2012). Among the 6 VvFLS genes in grape, only one was expressed in the grape cell culture, identical to the FLS gene overexpressed in the transgenic AroG* + FLS lines. The expression level of four R2R3-MYB transcription factors was analyzed, MYB14 and MYB15 that regulate stilbene biosynthesis (Holl et al., 2013), and MYBPA and MYBA that regulate flavonoid biosynthesis in V. vinifera (Czemmel et al., 2012).

AroG* overexpression had a relatively minor effect on the gene expression levels, with a decrease in the expression of several genes such as STS10 and MYB14 (FIG. 18B). The one gene that was induced dramatically (15-20-fold) due to the introduction of the AroG* transgene was F3′5′H, directing flavonoid biosynthesis towards the delphinidin-related anthocyanins. This induction correlates to the increased levels of the malvidin anthocyanins in this transgenic line (FIG. 18A). The expression levels of the three STS genes were lower in the AroG* line.

The gene expression pattern differed with the additional overexpression of FLS. Co-expression of AroG* and FLS caused significant induction of genes along the phenylpropanoid pathway, including PAL, C4H and 4CL, as well as many genes along the anthocyanin biosynthesis pathway (FIG. 18B). This correlates with the increase in flavonoids and anthocyanins in this line. The induction in LAR is in direct correlation to the increased levels of several flavan-3-ols in this AroG*+FLS-22 line (FIG. 18B). Here too, apart for a small increase in MYB15, there was no significant induction of STS genes despite the dramatic increase in stilbenes levels.

Feeding with exogenous Phe had a minor effect on gene expression levels in the control line, including an induction of the F3′5′H gene, correlating to an increase in the delphinidin-related anthocyanins due to this feeding (FIG. 18B). The most significant effect of Phe feeding was on the AroG* line, resulting in induction of PAL, C4H and 4CL as well several genes along the flavonoid and anthocyanin pathway, to levels similar to the non-fed AroG* + FLS cells.

These results demonstrate a significant increase in both stilbenes and flavonoids in the grape cell culture by transforming the cell with both AroG*, for increased Phe production, and FLS, for diverting the carbon flux towards flavonoids biosynthesis. This co-expression and addition of exogenous Phe resulted in grape cells rich in both groups of health-promoting compounds (FIG. 18A). When Phe availability in the V. vinifera cv. Gamay Red cell culture was high by overexpression of AroG* plus external Phe feeding, stilbene levels increased dramatically, while the increase in flavonoids was minor (FIG. 18A). However, co-expression of AroG* + FLS resulted in similar stilbene levels as compared to cell lines transformed only with AroG* but with additionally increased accumulation of flavonoids. This demonstrates an increase in the total carbon flux toward both stilbenes and flavonoids biosynthesis, in the AroG* + FLS lines, in comparison to the AroG* line (FIG. 18, Table 3).

In conclusion, increasing the availability of Phe and overexpressing FLS, increased the total carbon flow towards phenylpropanoid production, resulting in enhanced levels in both health-promoting metabolic groups of stilbenes and flavonoids in V. vinifera cv. Gamay Red cell suspension.

### REFERENCES

Aleynova, O.A., Grigorchuk, V.P., Dubrovina, A.S., Rybin, V.G. and Kiselev, K.V. (2016) Stilbene accumulation in cell cultures of Vitis amurensis Rupr. overexpressing VaSTS1, VaSTS2, and VaSTS7 genes. Plant Cell, Tissue and Organ Culture (PCTOC) 125, 329-339.

Anesi, A.; Stocchero, M.; Dal Santo, S.; Commisso, M.; Zenoni, S.; Ceoldo, S.; Tornielli, G. B.; Siebert, T. E.; Herderich, M.; Pezzotti, M.; Guzzo, F., Towards a scientific interpretation of the terroir concept: plasticity of the grape berry metabolome. BMC Plant Biol 2015, 15, 191.

Babu, P. V. A.; Liu, D., Chapter 18 - Flavonoids and Cardiovascular Health. In Complementary and Alternative Therapies and the Aging Population, Watson, R. R., Ed. Academic Press: San Diego, 2009; pp 371-392.

Baur, J.A. and Sinclair, D.A. (2006) Therapeutic potential of resveratrol: the in vivo evidence. Nature reviews. Drug discovery 5, 493-506.

Blancquaert, E. H.; Oberholster, A.; Ricardo-da-Silva, J. M.; Deloire, A. J., Grape Flavonoid Evolution and Composition Under Altered Light and Temperature Conditions in Cabernet Sauvignon (Vitis vinifera L.). Frontiers in plant science 2019, 10, 1062.

Chen, A. Y.; Chen, Y. C., A review of the dietary flavonoid, kaempferol on human health and cancer chemoprevention. Food Chemistry 2013, 138 (4), 2099-2107.

Chong, J., Poutaraud, A. and Hugueney, P. (2009) Metabolism and roles of stilbenes in plants. Plant Science 177, 143-155.

Chu, M., Pedreno, M.A., Alburquerque, N., Faize, L., Burgos, L. and Almagro, L. (2017) A new strategy to enhance the biosynthesis of trans-resveratrol by overexpressing stilbene synthase gene in elicited Vitis vinifera cell cultures. Plant physiology and biochemistry : PPB 113, 141-148.

Counet, C., Callemien, D. and Collin, S. (2006) Chocolate and cocoa: New sources of trans-resveratrol and trans-piceid. Food Chemistry 98, 649-657.

Czemmel, S.; Heppel, S. C.; Bogs, J., R2R3 MYB transcription factors: key regulators of the flavonoid biosynthetic pathway in grapevine. Protoplasma 2012, 249 (S2), 109-118.

Davies, K.M. and Deroles, S.C. (2014) Prospects for the use of plant cell cultures in food biotechnology. Current opinion in biotechnology 26, 133-140.

Feng, J.; Chen, X.; Wang, Y.; Du, Y; Sun, Q.; Zang, W.; Zhao, G., Myricetin inhibits proliferation and induces apoptosis and cell cycle arrest in gastric cancer cells. Molecular and Cellular Biochemistry 2015, 408 (1), 163-170.Ahuja, I., Kissen, R. and Bones, A.M. (2012) Phytoalexins in defense against pathogens. Trends in plant science 17, 73-90.

Fujita, A., Goto-Yamamoto, N., Aramaki, I., & Hashizume, K. , Organ-specific transcription of putative flavonol synthase genes of grapevine and effects of plant hormones and shading on flavonol biosynthesis in grape berry skins. Biosci Biotechnol Biochem 2006, 70(3), 632-638.

Gibson, D. G.; Young, L.; Chuang, R.-Y.; Venter, J. C.; Hutchison, C. A.; Smith, H. O., Enzymatic assembly of DNA molecules up to several hundred kilobases. Nature methods 2009, 6 (5), 343-345.

Gindro, K., Spring, J., Pezet, R., Richter, H. and Viret, O. (2006) Histological and biochemical criteria for objective and early selection of grapevine cultivars resistant to Plasmopara viticola. VITIS-GEILWEILERHOF- 45, 191.

Haigis, M. C.; Sinclair, D. A., Mammalian Sirtuins: Biological Insights and Disease Relevance. Annual Review of Pathology: Mechanisms of Disease 2010, 5 (1), 253-295.

Harbome, J. B.; Williams, C. A., Advances in flavonoid research since 1992. Phytochemistry 2000, 55 (6), 481-504.

Hidalgo, D., Martínez-Márquez, Ascensión, Cusidó, Rosa, Bru-Martínez, Roque, Palazón, Javier, Corchete, Purificación (2017) Silybum marianum cell cultures stably transformed with Vitis vinifera stilbene synthase accumulate t-resveratrol in the extracellular medium after elicitation with methyl jasmonate or methylated βcyclodextrins. Engineering in Life Sciences 17, 686-694.

Holl, J.; Vannozzi, A.; Czemmel, S.; D′Onofrio, C.; Walker, A. R.; Rausch, T.; Lucchin, M.; Boss, P. K.; Dry, I. B.; Bogs, J., The R2R3-MYB transcription factors MYB14 and MYB15 regulate stilbene biosynthesis in Vitis vinifera. The Plant cell 2013, 25 (10), 4135-49.

Hollman, P. C. H.; Katan, M. B., Dietary Flavonoids: Intake, Health Effects and Bioavailability. Food and Chemical Toxicology 1999, 37 (9), 937-942.

Jiang, X.; Shi, Y.; Fu, Z.; Li, W.—W.; Lai, S.; Wu, Y.; Wang, Y.; Liu, Y.; Gao, L.; Xia, T, Functional characterization of three flavonol synthase genes from Camellia sinensis: Roles in flavonol accumulation. Plant Science 2020, 300, 110632.

Kalantari, H. and Das, D.K. (2010) Physiological effects of resveratrol. BioFactors 36, 401-406.

Kiselev, K. V.; Shumakova, O. A.; Manyakhin, A. Y, Effect of plant stilbene precursors on the biosynthesis of resveratrol in Vitis amurensis Rupr. Cell cultures. Applied Biochemistry and Microbiology 2013, 49 (1), 53-58.

Kiselev, K.V and Aleynova, O.A. (2016) Influence of overexpression of stilbene synthase VaSTS7 gene on resveratrol production in transgenic cell cultures of grape Vitis amurensis Rupr. Applied Biochemistry and Microbiology 52, 56-60.

Kris-Etherton, P. M.; Lefevre, M.; Beecher, G. R.; Gross, M. D.; Keen, C. L.; Etherton, T. D., BIOACTIVE COMPOUNDS IN NUTRITION AND HEALTH-RESEARCH METHODOLOGIES FOR ESTABLISHING BIOLOGICAL FUNCTION: The Antioxidant and Anti-inflammatory Effects of Flavonoids on Atherosclerosis. Annual Review of Nutrition 2004, 24 (1), 511-538.

Langcake, P. (1981) Disease resistance of Vitis spp. and the production of the stress metabolites resveratrol, ε-viniferin, α-viniferin and pterostilbene. Physiological Plant Pathology 18, 213-226.

Manela, N., Oliva, M., Ovadia, R., Sikron-Persi, N., Ayenew, B., Fait, A., Galili, G., Perl, A., Weiss, D. and Oren-Shamir, M. (2015) Phenylalanine and tyrosine levels are rate-limiting factors in production of health promoting metabolites in Vitis vinifera cv. Gamay Red cell suspension. Frontiers in plant science 6, 538.

Nielsen Karen; Simon C. Deroles; Kenneth R. Markham; Marie J. Bradley; Ellen Podivinsky; Manson., D., Antisense flavonol synthase alters copigmentation and flower color in lisianthus. Molecular Breeding 2002, 9(4), 217-229.

Oliva, M., Ovadia, R., Perl, A., Bar, E., Lewinsohn, E., Galili, G. and Oren-Shamir, M. (2015) Enhanced formation of aromatic amino acids increases fragrance without affecting flower longevity or pigmentation in Petunia x hybrida. Plant biotechnology journal 13, 125-136.

Owens, D. K.; Alerding, A. B.; Crosby, K. C.; Bandara, A. B.; Westwood, J. H.; Winkel, B. S., Functional analysis of a predicted flavonol synthase gene family in Arabidopsis. Plant physiology 2008, 147 (3), 1046-61.

Parage, C., Tavares, R., Rety, S., Baltenweck-Guyot, R., Poutaraud, A., Renault, L., Heintz, D., Lugan, R., Marais, G.A., Aubourg, S. and Hugueney, P. (2012) Structural, functional, and evolutionary analysis of the unusually large stilbene synthase gene family in grapevine. Plant physiology 160, 1407-1419.

Pezet, R., Gindro, K., Viret, O. and Spring, J.L. (2004) Glycosylation and oxidative dimerization of resveratrol are respectively associated to sensitivity and resistance of grapevine cultivars to downy mildew. Physiological and Molecular Plant Pathology 65, 297-303.

Piver, B., Berthou, F., Dreano, Y. and Lucas, D. (2003) Differential inhibition of human cytochrome P450 enzymes by s-viniferin, the dimer of resveratrol: comparison with resveratrol and polyphenols from alcoholized beverages. Life Sciences 73, 1199-1213.

Rafe, T., Shawon, P. A., Salem, L., Chowdhury, N. I., Kabir, F., Bin Zahur, S. M., ... & Sagor, M. A. (2019). Preventive role of Resveratrol against inflammatory cytokines and related diseases. Current pharmaceutical design, 25(12), 1345-1371.

Rimando, A.M., Pan, Z., Polashock, J.J., Dayan, F.E., Mizuno, C.S., Snook, M.E., Liu, C.J. and Baerson, S.R. (2012) In planta production of the highly potent resveratrol analogue pterostilbene via stilbene synthase and O-methyltransferase co-expression. Plant biotechnology journal 10, 269-283.

Schnee, S., Queiroz, E.F., Voinesco, F., Marcourt, L., Dubuis, P.H., Wolfender, J.L. and Gindro, K. (2013) Vitis vinifera canes, a new source of antifungal compounds against Plasmopara viticola, Erysiphe necator, and Botrytis cinerea. Journal of agricultural and food chemistry 61, 5459-5467.

Shen, T., Wang, X.—N. and Lou, H.-X. (2009) Natural stilbenes: an overview. Natural product reports 26, 916-935.

Suprun, A.R., Ogneva, Z.V., Dubrovina, A.S. and Kiselev, K.V. (2019) Effect of spruce PjSTS1a, PjSTS2, or PjSTS3 gene overexpression on stilbene biosynthesis in callus cultures of Vitis amurensis Rupr. Biotechnology and applied biochemistry.

Tzin, V., Malitsky, S., Ben Zvi, M.M., Bedair, M., Sumner, L., Aharoni, A. and Galili, G. (2012) Expression of a bacterial feedback-insensitive 3-deoxy-D-arabino-heptulosonate 7-phosphate synthase of the shikimate pathway in Arabidopsis elucidates potential metabolic bottlenecks between primary and secondary metabolism. The New phytologist 194, 430-439.

Tzin, V., Rogachev, I., Meir, S., Moyal Ben Zvi, M., Masci, T., Vainstein, A., Aharoni, A. and Galili, G. (2015) Altered Levels of Aroma and Volatiles by Metabolic Engineering of Shikimate Pathway Genes in Tomato Fruits. AIMS Bioengineering 2, 75-92.

Vannozzi Alessandro, I. B. D., Marianna Fasoli, Sara Zenoni and Margherita Lucchin, Genome-wide analysis of the grapevine stilbene synthase multigenic family: genomic organization and expression profiles upon biotic and abiotic stresses. BMC Plant Biology 2012, 12.1: 130.

Vion, E., Page, G., Bourdeaud, E., Paccalin, M., Guillard, J. and Rioux Bilan, A. (2018) Trans epsilon-viniferin is an amyloid-beta disaggregating and anti-inflammatory drug in a mouse primary cellular model of Alzheimer’s disease. Molecular and cellular neurosciences 88, 1-6.

Vitrac, X., Bornet, A., Vanderlinde, R., Valls, J., Richard, T., Delaunay, J.—C., Merillon, J.-M. and Teissedre, P.-L. (2005) Determination of stilbenes (8-viniferin, trans-astringin, trans-piceid, cis-and trans-resveratrol, ε-viniferin) in Brazilian wines. Journal of agricultural and food chemistry 53, 5664-5669.

Vu, T. T.; Jeong, C. Y.; Nguyen, H. N.; Lee, D.; Lee, S. A.; Kim, J. H.; Hong, S. W.; Lee, H., Characterization of Brassica napus Flavonol Synthase Involved in Flavonol Biosynthesis in Brassica napus L. Journal of agricultural and food chemistry 2015, 63 (35), 7819-29.

Wang, R.; Lenka, S. K.; Kumar, V.; Gashu, K.; Sikron-Persi, N.; Dynkin, I.; Weiss, D.; Perl, A.; Fait, A.; Oren-Shamir, M., Metabolic Engineering Strategy Enables a Hundred-Fold Increase in Viniferin Levels in Vitis vinifera cv. Gamay Red Cell Culture. Journal of agricultural and food chemistry 2021, 69 (10), 3124-3133.

Yin, X., Singer, S.D., Qiao, H., Liu, Y., Jiao, C., Wang, H., Li, Z., Fei, Z., Wang, Y, Fan, C. and Wang, X. (2016) Insights into the Mechanisms Underlying Ultraviolet-C Induced Resveratrol Metabolism in Grapevine (V. amurensis Rupr.) cv. “Tonghua-3”. Frontiers in plant science 7, 503.

Zghonda, N., Yoshida, S., Ezaki, S., Otake, Y, Murakami, C., Mliki, A., Ghorbel, A. and Miyazaki, H. (2012) epsilon-Viniferin is more effective than its monomer resveratrol in improving the functions of vascular endothelial cells and the heart. Bioscience, biotechnology, and biochemistry 76, 954-960.

1. A doubly-transgenic Vitis Vinifera cell, comprising:
(i) at least one copy of an AroG* gene, and
(ii) at least one copy of a stilbene synthase (STS) gene or a flavonol synthase (FLS) gene.

2. The cell of claim 1, wherein the Vitis Vinifera cell is a Vitis Vinifera cv. Gamay Red cell.

3. The cell of claim 1, wherein the AroG* gene encodes a 3-Deoxy-D-arabinoheptulosonate 7-phosphate (DAHP) synthase (DAHPS) enzyme, wherein
(a) the DAHPS enzyme is a feedback-insensitive DAHPS enzyme; or
(b) the DAHPS enzyme increases the availability of at least one amino-acid in the cell; or
(c) the DAHPS enzyme increases the availability of Phenylalanine in the cell; or
(d) a combination thereof.

4-6. (canceled)

7. The cell of claim 1, wherein
(a) the STS gene encodes an STS enzyme; or
(b) the STS enzyme produces a stilbene; or
(c) the STS gene is a Vitis vinifera stilbene synthase (VvSTS) gene: or
(d) the STS gene is selected from the group consisting of VvSTS5, VvSTS10 and VvSTS28; or
(e) a combination thereof.

8-10. (canceled)

11. The cell of claim 1, wherein
(a) the FLS gene encodes an FLS enzyme; or
(b) the FLS enzyme produces a flavonoid; or
(c) the FLS gene is a Vitis vinifera flavonol synthase (VvFLS) gene: or
(d) the FLS gene is VIT 07s0031g00100; or
(e) a combination thereof.

12-14. (canceled)

15. The cell of claim 1, wherein when said cell comprises at least one copy of an AroG* gene, and at least one copy of a STS gene:
(a) the AroG* gene or the STS gene is functionally-linked to a constitutive promoter; or
(b) the AroG* gene and the STS gene are both functionally-linked to a constitutive promoter; or
(c) the AroG* gene and the STS gene are functionally-linked to different constitutive promoters; or wherein
when said cell comprises at least one copy of an AroG* gene, and at least one copy of a FLS gene: (a) the AroG* gene or the FLS gene is functionally-linked to a constitutive promoter;
or
(b) the AroG* gene and the FLS gene are both functionally-linked to a constitutive promoter; or
(c) the AroG* gene and the STS gene are functionally-linked to different constitutive promoters.

16-21. (canceled)

22. The cell of claim 1, comprising a higher level of:
(i) at least one amino-acid selected from the group consisting of Phenylalanine, Tryptophan, and p-CA;
(ii) at least one stilbene selected from the group consisting of trans-piceid, cis-piceid, resveratrol, and ε-viniferin;
(iii) at least one flavonoid selected from the group consisting of quercetin-3-glucoside, myricetin, catechin, epicatechin, epigallocatechin, procyanidin B1 and procyanidin B2;
(iv) at least one anthocyanin selected from the group consisting of cyanidin, peonidin, delphinidin, petunidin, and malvidin ; or
(v) any combination of (i), (ii), (iii), and (iv),
compared to a corresponding non-transgenic Vitis Vinifera cell or compared to a corresponding singly-transgenic Vitis Vinifera cell.

23. The cell of claim 1, comprising:
(i) a concentration of at least 0.5 mg/g dry weight trans-piceid,
(ii) a concentration of at least 0.5 mg/g dry weight cis-piceid,
(iii) a concentration of at least 0.8 mg/g dry weight resveratrol,
(iv) a concentration of at least 0.6 mg/g dry weight ε-viniferin, or
(v) any combination of (i), (ii), (iii), and (iv).

24. (canceled)

25. A method for maintaining a Vitis Vinifera cell optionally comprising at least one copy of an AroG* gene, optionally comprising at least one copy of a stilbene synthase (STS) gene, and optionally comprising at least one copy of a flavonol synthase (FLS) gene, the method comprising contacting the cell with a composition comprising:
(a) phenylalanine in a concentration of about 0.2 mM to about 5 mM,
(b) p-coumaric acid in a concentration of about 0.1 mM to about 0.3 mM, or
(c) any combination of (a) and (b).

26-28. (canceled)

29. The method of claim 25, comprising contacting the cell with a composition comprising:
(a) phenylalanine in a concentration of about 2 mM to about 5 mM; or
(b) phenylalanine in a concentration of about 5 mM; or
(c) p-coumaric acid in a concentration of about 0.3 mM; or
(d) both (a) and (c) or (b) and (c).

30. (canceled)

31. (canceled)

32. A pharmaceutical composition, comprising a doubly-transgenic Vitis Vinifera cell according to claim 1, or an extract or fraction thereof.

33. The pharmaceutical composition of claim 32, wherein the doubly-transgenic Vitis Vinifera cell was maintained by the method of claim 25 .

34. A pharmaceutical composition, comprising a non-transgenic Vitis Vinifera cell or a single-transgenic Vitis Vinifera cell comprising at least one copy of an AroG* gene, wherein the cell was maintained by the method of claim 25, or an extract or fraction thereof.

35. The pharmaceutical composition of claim 32 34, comprising:
(i) at least one amino-acid selected from the group consisting of Phenylalanine, Tryptophan, and p-CA;
(ii) at least one stilbene selected from the group consisting of trans-piceid, cis-piceid, resveratrol, and ε-viniferin;
(iii) at least one flavonoid selected from the group consisting of quercetin-3-glucoside, myricetin, catechin, epicatechin, epigallocatechin, procyanidin B1 and procyanidin B2;
(iv) at least one anthocyanin selected from the group consisting of cyanidin, peonidin, delphinidin, petunidin, and malvidin ; or
(v) any combination of (i), (ii), (iii), and (iv).

36. The pharmaceutical composition of claim 32 , comprising:
(a) an extract of the doubly-transgenic Vitis Vinifera cell ; or
(b) a cytoplasmic fraction of the doubly-transgenic Vitis Vinifera cell ; or
(c) a polyphenolic fraction of the doubly-transgenic Vitis Vinifera cell ; or
(d) the vacuole of the doubly-transgenic Vitis Vinifera cell .

37-39. (canceled)

40. The pharmaceutical composition of claim 32 , being
(a) substantially dehydrated; or
(b) substantially devoid of intact cells; or
(c) substantially devoid of ruptured cells; or
(d) both (a) and (b) or (a) and (c).

41-50. (canceled)

51. A method of treating, preventing, ameliorating, inhibiting, or reducing the incidence of a cytokine release syndrome (CRS) or a Cytokine Storm in a patient in need, the method comprising administering to the patient a therapeutically-effective amount of the pharmaceutical composition of claim 32.

52. The method of claim 51, wherein the CRS or Cytokine Storm is associated with a Coronavirus infection or with a symptom thereof.

53. The method of claim 52, wherein the Coronavirus infection comprises Severe acute respiratory syndrome coronavirus 2 (SARS-CoV-2) infection.

54-59. (canceled)

60. An edible or a potable composition, comprising the pharmaceutical composition of claim 32.

61. (canceled)
